SEQUENCE LISTING (138 sequences)

SEQ ID NO: 1 - protein, 24 aa, Synthetic Construct
GHSVDRIPEYFGPPGLPGPVLFYS

SEQ ID NO: 2 - protein, 28 aa, Synthetic Construct
GGDGSIAEYFNRPMHIHDWQIMDSGYYG

SEQ ID NO: 3 - protein, 21 aa, Synthetic Construct
GGAGHVPEYFVGIGTPISFYG

SEQ ID NO: 4 - protein, 19 aa, Synthetic Construct
GGVGKIIEYFIGGGVGRYG

SEQ ID NO: 5 - protein, 19 aa, Synthetic Construct
GSDGPIIEFFNPNGVMHYG

SEQ ID NO: 6 - protein, 18 aa, Synthetic Construct
GGKGPIFETWVTEGNYYG

SEQ ID NO: 7 - protein, 10 aa, Synthetic Construct
GHSVADRIPE

SEQ ID NO: 8 - protein, 15 aa, Synthetic Construct
  SITE 1: note = Residue 1 (Y) is amine terminus bonded to the carboxy terminus of Residue 9 (E) in SEQ ID NO. 138
YFGPPGLPGPVLFYS

SEQ ID NO: 9 - protein, 9 aa, Synthetic Construct
GHSVDRIPE

SEQ ID NO: 10 - protein, 11 aa, Synthetic Construct
GPPGLPGPVLF

SEQ ID NO: 11 - protein, 11 aa, Synthetic Construct
FGPPGLPGPVL

SEQ ID NO: 12 - protein, 11 aa, Synthetic Construct
  SITE 1: note = Residue 1 (Y) is amine terminus bonded to the carboxy terminus of Residue 9 (E) of SEQ ID NO. 66
YFGPPGLPGPV

SEQ ID NO: 13 - protein, 56 aa, Synthetic Construct
MKNKHALTQRNEKALNAVSITRIPVKASKITRGHSVDRIPEYFGPPGLPGPVLFYS

SEQ ID NO: 14 - protein, 58 aa, Synthetic Construct
MDVKNKHALTQRNEKALNAVSITRIPVKASKITRGHSVDRIPEYFGPPGLPGPVLFYS

SEQ ID NO: 15 - protein, 56 aa, Synthetic Construct
MKNKNALNQRNEKPVNAVSINRIPVKASKITRGHSVDRIPEYFGPPGLPGPVLFYS

SEQ ID NO: 16 - protein, 56 aa, Synthetic Construct
MKNKNALNQRNEKTVNAVSINRIPVKASKITRGHSVDRIPEYFGPPGLPSPVLFYS

SEQ ID NO: 17 - protein, 56 aa, Synthetic Construct
MKNKNALNQRNEKTVNAVSINRIPVKASKITRGHSVDRIPEYFGPPGLPGPVLFYS

SEQ ID NO: 18 - protein, 58 aa, Synthetic Construct
MDVKNKAVLNQRNEKALNAVSIKRIPVKASKITRGHSVDRIPEYFGPPGLPGPVLFYS

SEQ ID NO: 19 - protein, 56 aa, Synthetic Construct
MKNKAVLNQRNEKALNAVSIKRIPVKASKITRGHSVDRIPEYFGPPGLPGPVLFYS

SEQ ID NO: 20 - protein, 58 aa, Synthetic Construct
MDMKNQHALNQRKDNTVNAVSINRIPVKASKITRGHSVDRIPEYFGPPGVPGPVLFYS

SEQ ID NO: 21 - protein, 58 aa, Synthetic Construct
MDHKNQHALNQRKENTVNPVSINRIPVKASKITRGHSVDRIPEYFGPPGLPGPVLFYS

SEQ ID NO: 22 - protein, 56 aa, Synthetic Construct
MKNQHALNQRKENTVNPVSINRIPVKASKITRGHSVDRIPEYFGPPGLPGPVLFYS

SEQ ID NO: 23 - protein, 58 aa, Synthetic Construct
MDMKNKHALNQRKDNTFDAVSINRIPVKASKITRGHSVDRIPEYFGPPGLPGPVLFYS

SEQ ID NO: 24 - protein, 58 aa, Synthetic Construct
MDMRNQHALNQRKENTDNPVSINRIPVKASKITRGHSVDRIPEYFGPPGLPSPVLFYS

SEQ ID NO: 25 - protein, 56 aa, Synthetic Construct
MRNQHALNQRKENTDNPVSINRIPVKASKITRGHSVDRIPEYFGPPGLPSPVLFYS

SEQ ID NO: 26 - protein, 54 aa, Synthetic Construct
MKANVLPVVHVTKKDNTVSVNRIPTKASKITRGHSVDRIPEYFGPPGGPILFYS

SEQ ID NO: 27 - protein, 15 aa, Synthetic Construct
GHSVDRIPEYFGPPG

SEQ ID NO: 28 - protein, 14 aa, Synthetic Construct
GHSVDRIPEYFGPP

SEQ ID NO: 29 - protein, 18 aa, Synthetic Construct
GHSVDRIPEYFGPPGLPG

SEQ ID NO: 30 - protein, 9 aa, Synthetic Construct
  SITE 1: note = Residue 1 (Y) is amine terminus bonded to the carboxy terminus of Residue 9 (E) in SEQ ID NO. 121
YFGPPGLPG

SEQ ID NO: 31 - protein, 6 aa, Synthetic Construct
  SITE 1: note = Residue 1 (Y) is amine terminus bonded to the carboxy terminus of Residue 9 (E) in SEQ ID NO. 122
YFGPPG

SEQ ID NO: 32 - protein, 5 aa, Synthetic Construct
  SITE 1: note = Residue 1 (Y) is amine terminus bonded to the carboxy terminus of Residue 9 (E) in SEQ ID NO. 123
YFGPP

SEQ ID NO: 33 - protein, 6 aa, Synthetic Construct
PVLFYS

SEQ ID NO: 34 - protein, 7 aa, Synthetic Construct
GPVLFYS

SEQ ID NO: 35 - protein, 8 aa, Synthetic Construct
PGPVLFYS

SEQ ID NO: 36 - protein, 9 aa, Synthetic Construct
LPGPVLFYS

SEQ ID NO: 37 - protein, 12 aa, Synthetic Construct
PPGLPGPVLFYS

SEQ ID NO: 38 - protein, 13 aa, Synthetic Construct
GPPGLPGPVLFYS

SEQ ID NO: 39 - protein, 14 aa, Synthetic Construct
FGPPGLPGPVLFYS

SEQ ID NO: 40 - protein, 10 aa, Synthetic Construct
GHSVDRIPEY

SEQ ID NO: 41 - protein, 12 aa, Synthetic Construct
GHSVDRIPEYFG

SEQ ID NO: 42 - protein, 13 aa, Synthetic Construct
GHSVDRIPEYFGP

SEQ ID NO: 43 - protein, 16 aa, Synthetic Construct
GHSVDRIPEYFGPPGL

SEQ ID NO: 44 - protein, 20 aa, Synthetic Construct
GHSVDRIPEYFGPPGLPGPV

SEQ ID NO: 45 - protein, 21 aa, Synthetic Construct
GHSVDRIPEYFGPPGLPGPVL

SEQ ID NO: 46 - protein, 22 aa, Synthetic Construct
GHSVDRIPEYFGPPGLPGPVLF

SEQ ID NO: 47 - protein, 23 aa, Synthetic Construct
GHSVDRIPEYFGPPGLPGPVLFY

SEQ ID NO: 48 - protein, 12 aa, Synthetic Construct
GPPGLPGPVLFY

SEQ ID NO: 49 - protein, 13 aa, Synthetic Construct
FGPPGLPGPVLFY

SEQ ID NO: 50 - protein, 11 aa, Synthetic Construct
GHSVDRIPEYF

SEQ ID NO: 51 - protein, 11 aa, Synthetic Construct
GPPGLPGPVLF

SEQ ID NO: 52 - protein, 12 aa, Synthetic Construct
GPPGLPGPVLFY

SEQ ID NO: 53 - protein, 12 aa, Synthetic Construct
FGPPGLPGPVLF

SEQ ID NO: 54 - protein, 10 aa, Synthetic Construct
FGPPGLPGPV

SEQ ID NO: 55 - protein, 12 aa, Synthetic Construct
FGPPGLPGPVLF

SEQ ID NO: 56 - protein, 14 aa, Synthetic Construct
FGPPGLPGPVLFYS

SEQ ID NO: 57 - protein, 10 aa, Synthetic Construct
FGPPGLPGPV

SEQ ID NO: 58 - protein, 11 aa, Synthetic Construct
FGPPGLPGPVL

SEQ ID NO: 59 - protein, 14 aa, Synthetic Construct
FGPPGLPGPVLFYS

SEQ ID NO: 60 - protein, 4 aa, Synthetic Construct
LFYS

SEQ ID NO: 61 - protein, 15 aa, Synthetic Construct
GHSVDRIPEYFGPPG

SEQ ID NO: 62 - protein, 14 aa, Synthetic Construct
GHSVDRIPEYFGPP

SEQ ID NO: 63 - protein, 20 aa, Synthetic Construct
GHSVDRIPEYFGPPGPGLPG

SEQ ID NO: 64 - protein, 24 aa, Synthetic Construct
GHSVDRIPEYFGPPGLPGPVLFYG

SEQ ID NO: 65 - protein, 24 aa, Synthetic Construct
GHSVDRIPEYFGPPGLPGPVLFYG

SEQ ID NO: 66 - protein, 9 aa, Synthetic Construct
  SITE 9: note = Residue 9 (E) is carboxy terminus bonded to the amine terminus of Residue 1 (Y) of SEQ ID NO. 12
  SITE 9: note = Residue 9 (E) is side chain carboxy bonded to the amine terminus of Residue 1 (G) of SEQ ID NO. 66
  SITE 1: note = Residue 1 (G) is amine terminus bonded to the side chain carboxy of Residue 9 (E) of SEQ ID NO. 66
GHSVDRIPE

SEQ ID NO: 67 - protein, 25 aa, Synthetic Construct
GHSVADRIPEYFGPPGLPGPVLFYS

SEQ ID NO: 68 - DNA, 41 bp, Synthetic Construct
  source 1..41: mol_type = other DNA
cgatcgtgaattcattaaagaggagaaattaactatggatg

SEQ ID NO: 69 - DNA, 32 bp, Synthetic Construct
  source 1..32: mol_type = other DNA
cgatgcaagcttttagctccaaaacaggactg

SEQ ID NO: 70 - DNA, 33 bp, Synthetic Construct
  source 1..33: mol_type = other DNA
cgatgcaagcttttagctataccacaggactgg

SEQ ID NO: 71 - DNA, 30 bp, Synthetic Construct
  source 1..30: mol_type = other DNA
tgacgaagcttttagccataaaacaggact

SEQ ID NO: 72 - DNA, 33 bp, Synthetic Construct
  source 1..33: mol_type = other DNA
acagaaaagcttttaggtataaaacaggactgg

SEQ ID NO: 73 - DNA, 32 bp, Synthetic Construct
  source 1..32: mol_type = other DNA
aagcaaaagcttttagcaataaaacaggactg

SEQ ID NO: 74 - DNA, 34 bp, Synthetic Construct
  source 1..34: mol_type = other DNA
aagcaaaagcttttaggcataaaacaggactgga

SEQ ID NO: 75 - DNA, 35 bp, Synthetic Construct
  source 1..35: mol_type = other DNA
acagaaaagcttttaataataaaacaggactggac

SEQ ID NO: 76 - DNA, 33 bp, Synthetic Construct
  source 1..33: mol_type = other DNA
acagaaaagcttttaccaataaaacaggactgg

SEQ ID NO: 77 - DNA, 33 bp, Synthetic Construct
  source 1..33: mol_type = other DNA
acagaaaagcttttaaaaataaaacaggactgg

SEQ ID NO: 78 - DNA, 31 bp, Synthetic Construct
  source 1..31: mol_type = other DNA
gctaattaagcttttagctataaaacaggac

SEQ ID NO: 79 - DNA, 20 bp, Synthetic Construct
  source 1..20: mol_type = other DNA
acgatggatccgtcgtgagg

SEQ ID NO: 80 - DNA, 41 bp, Synthetic Construct
  source 1..41: mol_type = other DNA
ggcataccatggttaaacaaactcaaaaaattcatgccttg

SEQ ID NO: 81 - DNA, 33 bp, Synthetic Construct
  source 1..33: mol_type = other DNA
acgctatgggcacgtgtaattttgctcgctttc

SEQ ID NO: 82 - DNA, 25 bp, Synthetic Construct
  source 1..25: mol_type = other DNA
cgtgcccatagcgtggatcgtattc

SEQ ID NO: 83 - DNA, 23 bp, Synthetic Construct
  source 1..23: mol_type = other DNA
gtagtccggaatacgatccacgc

SEQ ID NO: 84 - DNA, 32 bp, Synthetic Construct
  source 1..32: mol_type = other DNA
ggatcgtattccggactactttggccctccgg

SEQ ID NO: 85 - DNA, 31 bp, Synthetic Construct
  source 1..31: mol_type = other DNA
gagggccaaaggcctccggaatacgatccac

SEQ ID NO: 86 - DNA, 20 bp, Synthetic Construct
  source 1..20: mol_type = other DNA
ggaggcctttggccctccgg

SEQ ID NO: 87 - DNA, 23 bp, Synthetic Construct
  source 1..23: mol_type = other DNA
ccgcaatacgatccacgctatgg

SEQ ID NO: 88 - DNA, 32 bp, Synthetic Construct
  source 1..32: mol_type = other DNA
cgtggatcgtattgcggagtactttggccctc

SEQ ID NO: 89 - DNA, 30 bp, Synthetic Construct
  source 1..30: mol_type = other DNA
cgatccgggctatggccacgtgtaattttg

SEQ ID NO: 90 - DNA, 28 bp, Synthetic Construct
  source 1..28: mol_type = other DNA
ccatagcccggatcgtattccggagtac

SEQ ID NO: 91 - DNA, 27 bp, Synthetic Construct
  source 1..27: mol_type = other DNA
aatacgatccgccacgctatggccacg

SEQ ID NO: 92 - DNA, 26 bp, Synthetic Construct
  source 1..26: mol_type = other DNA
cgtggcggatcgtattccggagtact

SEQ ID NO: 93 - DNA, 46 bp, Synthetic Construct
  source 1..46: mol_type = other DNA
cacacagaattcattaaagaggagaaattaactatggatgtgatga

SEQ ID NO: 94 - DNA, 47 bp, Synthetic Construct
  source 1..47: mol_type = other DNA
ggtcagcgcgtgtttattcttcatcacatccatagttaatttctcct

SEQ ID NO: 95 - DNA, 47 bp, Synthetic Construct
  source 1..47: mol_type = other DNA
agaataaacacgcgctgacccagcgtaatgaaaaagcgctgaatgcg

SEQ ID NO: 96 - DNA, 47 bp, Synthetic Construct
  source 1..47: mol_type = other DNA
ttcaccggtatacgggtgatgctcaccgcattcagcgctttttcatt

SEQ ID NO: 97 - DNA, 47 bp, Synthetic Construct
  source 1..47: mol_type = other DNA
catcacccgtataccggtgaaagcgagcaaaattacacgtggccata

SEQ ID NO: 98 - DNA, 47 bp, Synthetic Construct
  source 1..47: mol_type = other DNA
aagtactccggaatacgatccacgctatggccacgtgtaattttgct

SEQ ID NO: 99 - DNA, 47 bp, Synthetic Construct
  source 1..47: mol_type = other DNA
gtggatcgtattccggagtactttggccctccgggcctgccgggtcc

SEQ ID NO: 100 - DNA, 46 bp, Synthetic Construct
  source 1..46: mol_type = other DNA
ctaattaagcttttagctataaaacaggactggacccggcaggccc

SEQ ID NO: 101 - DNA, 1450 bp, Synthetic Construct
  source 1..1450: mol_type = other DNA
gcgttttttattggtgagaatccaagctagccatcaattaagaaaaaaatttagcttgtagataaattcagaagttttattattccaattgagtgtaaaggcataactacaggagggagtgtgcaaaatgctgattacgccggataccctgaaaattacctgctttaacaaggatctggtggtgcttgacttactgaaagatgaatataccgtgctggaaaataccagcgatagcttcctgacctttcagattccgaaccctgacgtagaggcgtttctgaatggcgaaaaaaactgccgttatctgcaggcggatagccacaatgatcccgaatcttttctggaagtgcgttggatgaaaccgcaggtgcgtattaaacattctagcgccctggctcgtatctcggcgctgcgtgcgatgctgtattgtcggcatggcctgaatcatggcggcttgattgcgctgaaacgtcagttttgcgaaagccagaccattggctgcgcgaccaaaatcactccggaagtggacattcgtatcaagaaagaaattgcggcgttgaatagcatttttgatctgattgatcacgataatccgtgcctggtctattcgtgcgcgcttaaacagcgcctgattagccgtaaaattggcgcaaccctggtgattggcgtgcgtacccgtccgttttatagccatgcgtgggtggaggtggatagcaccgtggtgagcgatgatccggacctgagaaagaaaacgagcgtcatactggagtgctgaccatgctgattgtgccagtgatgctgtccgaaagcgtggcagtgccggctcattttaccgctgtgcgtaccgggatgtacctgatttatctggacgcgggctggagctatcgggaagatgacggaaaactgtgcatttttagcggctgggcgattaccggcaatggcgaagtgttatcgttgagcgagaccgcacctgatgtgctggagaaagagcttaaaagcgccgatggctatttttgctttattgttatagaaaatgatgtgatacaagtaatgaagagcctgtatcgttattgcgatgtgtactacggcaagactgcgtttggcgctattattggcaccggcttgaaagatgtggcgcggattagcgggaccaaaaccattactcgggactttattcgtagcttcatacgggatagctttcaggcggcgtttctgagcccgctgcataataccggaaaggcgtactccggctgcttgacccggattgaccgtgaaaatatccataccgaagcgtttgtgaatctgacccctctgaagaaaaacaccctggacgtgattattacgattgtgaaaaaactgccagccgatctgccgctggcgttatatctgtccggcggattggatagcagcatcgtgttcttctgcctgctggctgcggataaaccgttta

SEQ ID NO: 102 - DNA, 1433 bp, Synthetic Construct
  source 1..1433: mol_type = other DNA
tgctggctgcggataaaccgtttaccgtgtttcattgcttaccgttcagctttgaaaccgacgcagaacgggcggacgcgcatcgtctgtgccgtaagtataacattaccctgaccgagctgaaacctggctataccaccgatcgccaaattagcaatcgtgtaaatcatccgagcgattgtccggtggtgaataccttttggctgaatgcgagtgacggcctgacacaagatatgagcgggtggttctgcttggacggtcatggcggcgacagcgtgtttattcagagcccgagcgtgcgcatcattcgtgacttaattcgcgtgggcaaaccgattcaagcgttgcggaccgcattccgtcttgcagcgttgaaaagccgctctttacacagcgtactggcgagcgcatacaaaaccgaaccggcgaaacgtacccatgttcatccccttctggtgggccatattccaggcaccgcatggtatgaatacttggaagaacttatccgtattagcgaaagcacagtgtcaatttttccggatggaccggcggtgaaatttagtcctcttattagcctgagcgtgattcgttcacgtctggcggtgcgttacgaggataacatacgtgctgggcatgatcgtgcggggatccgtcgtgaggcgctgacccgttttaaagatccggtgttcataaagaaaactaagcgttccagcagccaattgatttttacaattattcggcagtatgaggacgaactgattgcgttcgtggtccgttatggcacctatattctggacaaccctgaagcgatgattgaggaaattcgttataatgctcatgtggaattaaacggcagcctgccggaaattttgtccctgattaaactgatggtctttagccagaagatggaagctgattttttcgacgcggagtcaaggcatgaattttttgagtttgtttaaaaccgtgagccgtgcgaccggcacccgtcatgtgggcaaaattacgttcattatcctgctgagcgtgttaagcagcgtgctgatcataatttctccggtcctgattagtcgtacaaccggctacctgatgagcgataccgcggatagctttcgtgtgatatttaccttgggtgcggtgtatgtgtttgccatttcagcgcaaaagataattgctttttgggcgacctacctgcagagcgccctgcgcgtggaatgtattgcgagcataagcgagtttttccttaagaaactgtattatagcggtagccagaaggacaacgctggtggaattagccagcaacttaatcaagccaccaatgacatttacataatcatccgtgaactgaccttcggcctgttaccaccgctgattcagctgtttattgccctgtggaccattttagcgagcgatgatgtgattgtggcgtgcat

SEQ ID NO: 103 - DNA, 1293 bp, Synthetic Construct
  source 1..1293: mol_type = other DNA
tgatgtgattgtggcgtgcatgtttctgatgtttgcgattagctttgtgtttaccaattatgtgtatacccataaaatcgtggcggctcgtcaggcgctgatggacagcagccgtaaaacctacaccttattatccgattcggtccgtaatattccggtggtgcgggtgtttaacggctttggcgtgtttatgaagcgtttcaacggccagctgacagaggatgcgcgtacccagcgtagcttttggaaagttagcttccatgcgcagctggtgtcggcgttactgcagataatcttttttggctccgcgttcttttatactctgtatggcgcgagcaccggtcaggtgaccattgagcatttcattctggtgagcagctacattctgattttgagtgcccccttggaaaatctgggccagtcctatatcaattttcgtcagagtgctcatgcgtttagcgcatttagcggcggttggctggaagaaccggtggatcgtgatggcaatctgcgccgtccccaaaacggggatattactctgagcggcgtgtcatttcgttacccggaaagcccgagctgcacactgcatgacgtgaatttacacttccgcacgggcgagttcataaccattaccggcaagtccggttcaggcaaaagcaccctgttgaaaatcctggcacgtcagcttccgctgaccatagggaactatacactgggaggctgccctaatgaagaactgcctgagcaagtgttctacgaaaacattgcatatgtgagccaagaagaatatgtctttatggatagcgtggcgtataacttacagatcgcgaatccggaagcgactcgtgaagagatgatctcggccctggcgaaaagcggactgcaaattggacatctgcaaggcgaagcgctgctggatttgaacctggccaatgaaggcaccaacatttccggaggtcaacgccaacggctgagcttggcgcgtttgtttttgcgtaacccggcagtgatactgttggacgagattacctctagcctggatcttatatctgaaacccacgcaattaacgcgattttgtcagactttccgaccgcgaccattatttcgatctcgcatcgtataagcacctttaactatagcgatcgtattattgtgatgcataacggtcgggttagcgatgcaggccccctgaacgaactgctgcaacgtaacaacttcatagctcaactgcaccgtagcgaagaaagtccacctgtgaaagcgccatgggcaaatattatacgcaaggcgac

SEQ ID NO: 104 - DNA, 50 bp, Synthetic Construct
  source 1..50: mol_type = other DNA
gcgtataatatttgcccatggttacgctttcacaggtggactttcttcgc

SEQ ID NO: 105 - protein, 24 aa, Synthetic Construct
GHSVDRIPEYFGPPGLPSPVLFYS

SEQ ID NO: 106 - protein, 22 aa, Synthetic Construct
GHSVDRIPEYFGPPGGPILFYS

SEQ ID NO: 107 - protein, 56 aa, Synthetic Construct; identical to SEQ ID NO: 16

SEQ ID NO: 108 - protein, 54 aa, Synthetic Construct; identical to SEQ ID NO: 26

SEQ ID NO: 109 - protein, 24 aa, Synthetic Construct
GHSVDRIAEYFGPPGLPGPVLFYS

SEQ ID NO: 110 - protein, 24 aa, Synthetic Construct
GHSPDRIPEYFGPPGLPGPVLFYS

SEQ ID NO: 111 - protein, 24 aa, Synthetic Construct
GHSVDRIPEYFGPPGLPGPVLWYS

SEQ ID NO: 112 - protein, 24 aa, Synthetic Construct
GHSVDRIPEYFGPPGLPGPVLFWS

SEQ ID NO: 113 - protein, 24 aa, Synthetic Construct
GHSVDRIPEYFGPPGLPGPVLFYA

SEQ ID NO: 114 - protein, 24 aa, Synthetic Construct
GHSVDRIPEYFGPPGLPGPVLFYY

SEQ ID NO: 115 - protein, 13 aa, Synthetic Construct
YFGPPGLPGPVLF

SEQ ID NO: 116 - protein, 24 aa, Synthetic Construct
GHSVDRIPEYFGPPGLPGPVLFYT

SEQ ID NO: 117 - protein, 24 aa, Synthetic Construct
GHSVDRIPEYFGPPGLPGPVLFYC

SEQ ID NO: 118 - protein, 24 aa, Synthetic Construct
GHSVDRIPEAFGPPGLPGPVLFYS

SEQ ID NO: 119 - protein, 5 aa, Synthetic Construct
GHSVD

SEQ ID NO: 120 - protein, 4 aa, Synthetic Construct
RIPE

SEQ ID NO: 121 - protein, 9 aa, Synthetic Construct
  SITE 9: note = Residue 9 (E) is carboxy terminus bonded to the amine terminus of Residue 1 (Y) of SEQ ID NO. 30
  SITE 9: note = Residue 9 (E) is side chain carboxy bonded to the amine terminus of Residue 1 (G) in SEQ ID NO. 121
  SITE 1: note = Residue 1 (G) is amine terminus bonded to the side chain carboxy of Residue 9 (E) of SEQ ID NO. 121
GHSVDRIPE

SEQ ID NO: 122 - protein, 9 aa, Synthetic Construct
  SITE 9: note = Residue 9 (E) is carboxy terminus bonded to the amine terminus of Residue 1 (Y) of SEQ ID NO. 31
  SITE 9: note = Residue 9 (E) is side chain carboxy bonded to the amine terminus of Residue 1 (G) in SEQ ID NO. 122
  SITE 1: note = Residue 1 (G) is amine terminus bonded to the side chain carboxy of Residue 9 (E) of SEQ ID NO. 122
GHSVDRIPE

SEQ ID NO: 123 - protein, 9 aa, Synthetic Construct
  SITE 9: note = Residue 9 (E) is carboxy terminus bonded to the amine terminus of Residue 1 (Y) of SEQ ID NO. 32
  SITE 9: note = Residue 9 (E) is side chain carboxy bonded to the amine terminus of Residue 1 (G) in SEQ ID NO. 123
  SITE 1: note = Residue 1 (G) is amine terminus bonded to the side chain carboxy of Residue 9 (E) of SEQ ID NO. 123
GHSVDRIPE

SEQ ID NO: 124 - protein, 6 aa, Synthetic Construct
  SITE 1: note = Residue 1 (G) is amine terminus bonded to the side chain carboxy of Residue 3 (E) of SEQ ID NO. 125
GHSVDR

SEQ ID NO: 125 - protein, 18 aa, Synthetic Construct
  SITE 3: note = Residue 3 (E) is side chain carboxy bonded to the amine terminus of Residue 1 (G) of SEQ ID NO. 124
IPEYFGPPGLPGPVLFYS

SEQ ID NO: 126 - protein, 11 aa, Synthetic Construct
  SITE 9: note = Residue 9 (E) is side chain carboxy bonded to the amine terminus of Residue 1 (G) in SEQ ID NO. 126
  SITE 1: note = Residue 1 (G) is amine terminus bonded to the side chain carboxy of Residue 9 (E) of SEQ ID NO. 126
GHSVDRIPEYF

SEQ ID NO: 127 - protein, 10 aa, Synthetic Construct
  SITE 9: note = Residue 9 (E) is side chain carboxy bonded to the amine terminus of Residue 1 (G) in SEQ ID NO. 127
  SITE 1: note = Residue 1 (G) is amine terminus bonded to the side chain carboxy of Residue 9 (E) of SEQ ID NO. 127
GHSVDRIPEY

SEQ ID NO: 128 - protein, 10 aa, Synthetic Construct
GHSVDRIPEY

SEQ ID NO: 129 - protein, 15 aa, Synthetic Construct
GHSVDRIPEYFGPPG

SEQ ID NO: 130 - protein, 14 aa, Synthetic Construct
GHSVDRIPEYFGPP

SEQ ID NO: 131 - protein, 20 aa, Synthetic Construct
GHSVDRIPEYFGPPGPGLPG

SEQ ID NO: 132 - protein, 24 aa, Synthetic Construct
GHSVDRIPEYFGPPGLPGPVLFYG

SEQ ID NO: 133 - protein, 10 aa, Synthetic Construct
  SITE 9: note = Residue 10 (E) is carboxy terminus bonded to the amine terminus of Residue 1 (Y) of SEQ ID NO. 134
  SITE 9: note = Residue 10 (E) is side chain carboxy bonded to the amine terminus of Residue 1 (G) in SEQ ID NO. 133
  SITE 1: note = Residue 1 (G) is amine terminus bonded to the side chain carboxy of Residue 10 (E) of SEQ ID NO. 133
GHSVADRIPE

SEQ ID NO: 134 - protein, 15 aa, Synthetic Construct
  SITE 1: note = Residue 1 (Y) is amine terminus bonded to the carboxy terminus of Residue 10 (E) in SEQ ID NO. 133
YFGPPGLPGPVLFYS

SEQ ID NO: 135 - protein, 24 aa, Synthetic Construct
  SITE 9: note = Residue 9 (E) is side chain carboxy bonded to the amine terminus of Residue 1 (G) in SEQ ID NO. 135
  SITE 1: note = Residue 1 (G) is amine terminus bonded to the side chain carboxy of Residue 9 (E) of SEQ ID NO. 135
GHSVDRIPEYFGPPGLPGPVLFYS

SEQ ID NO: 136 - protein, 24 aa, Synthetic Construct
  SITE 9: note = Residue 9 (E) is side chain carboxy bonded to the amine terminus of Residue 1 (G) in SEQ ID NO. 136
  SITE 1: note = Residue 1 (G) is amine terminus bonded to the side chain carboxy of Residue 9 (E) of SEQ ID NO. 136
GHSVDRIPEYFGPPGLPSPVLFYS

SEQ ID NO: 137 - protein, 22 aa, Synthetic Construct
  SITE 9: note = Residue 9 (E) is side chain carboxy bonded to the amine terminus of Residue 1 (G) of SEQ ID NO. 137
  SITE 1: note = Residue 1 (G) is amine terminus bonded to the side chain carboxy of Residue 9 (E) of SEQ ID NO. 137
GHSVDRIPEYFGPPGGPILFYS

SEQ ID NO: 138 - protein, 9 aa, Synthetic Construct
  SITE 9: note = Residue 9 (E) is carboxy terminus bonded to the amine terminus of Residue 1 (Y) of SEQ ID NO. 8
  SITE 1: note = Residue 1 (G) is amine terminus bonded to the side chain carboxy of Residue 9 (E) of SEQ ID NO. 138
  SITE 9: note = Residue 9 (E) is side chain carboxy bonded to the amine terminus of Residue 1 (G) in SEQ ID NO. 138
GHSVDRIPE

(19) United States
(12) Patent Application Publication
(10) Pub. No.: US 2026/0092086 A1
(43) Pub. Date: Apr. 2, 2026

(54) ANTIMICROBIAL LASSO PEPTIDES

(71) Applicant: THE TRUSTEES OF PRINCETON UNIVERSITY, Princeton, NJ (US)

(72) Inventors: A. James LINK, Princeton, NJ (US); Drew CARSON, Princeton, NJ (US)

(73) Assignee: THE TRUSTEES OF PRINCETON UNIVERSITY, Princeton, NJ (US)

(21) Appl. No.: 19/112,242

(22) PCT Filed: Sep. 15, 2023
(86) PCT No.: PCT/US2023/074413

§ 371 Date: Mar. 14, 2025

Related U.S. Application Data
(60) Provisional application No. 63/407,392, filed on Sep. 16, 2022.

(51) Int. Cl. C07K 14/24; A61K 38/00; C12P 21/02

(52) U.S. Cl. CPC C07K 14/24; C12P 21/02; A61K 38/00

(57) ABSTRACT

Cloacaenodin is a lasso peptide resistant to cleavage by proteases and with antimicrobial properties, for example, with the ability to inhibit he growth of and kill Enterobacter species of bacteria.

This International Application claims the benefit of U.S. Provisional Application No. 63/407,392, filed Sep. 16, 2022, the specification of which is hereby incorporated by reference in its entirety.

This invention was made with government support under Grant No. GM107036 awarded by the National Institutes of Health. The government has certain rights in the invention.

An XML file for a Sequence Listing XML is submitted herewith.

### FIELD OF THE INVENTION

The rise in resistant bacterial infections jeopardizes the efficacy of the antibiotics developed in the 20^{th} and 21^{st} centuries. New compounds are needed to combat infections that were once treatable and have potential to cause loss of life.

### BRIEF SUMMARY OF THE INVENTION

A method of the invention for inhibiting growth of a microorganism, includes providing a cloacaenodin-class lasso peptide and exposing the microorganism to the cloacaenodin-class lasso peptide. This can inhibit the growth of the microorganism.

The cloacaenodin-class lasso peptide can be purified and/or isolated. The cloacaenodin-class lasso peptide can include a ring, a loop region, and a tail region. The ring can be bonded to the loop region: the loop region can be bonded to the tail region.

The cloacaenodin-class lasso peptide can include the peptide sequence GHSVDRIPEYFGPPGLPGPVLFYS [SEQ ID NO. 1]. Zero (0), one (1), or two (2) of residues 2 through 8 of the peptide sequence can be removed or replaced with another residue. Zero (0) or one (1) residue can be inserted after one of residues 1 through 8. Zero (0), one (1), two (2), three (3), four (4), or five (5) of residues 12 through 24 of the peptide sequence can be removed or replaced with another residue. Zero (0), one (1), two (2), three (3), or four (4) residues can be inserted after at least one of residues 11 through 21 (the inserted residues can be inserted after one residue or after several residues of residues 11 through 21).

In this text, a residue is an amino acid monomer that can be bonded to one or more other amino acids. A peptide sequence is numbered with the leftmost residue being the lowest numbered amino acid and numbering proceeding sequentially with each successive amino acid to the right. For example, for the cloacaenodin peptide sequence GHSVDRIPEYFGPPGLPGPVLFYS [SEQ ID NO. 1] the leftmost residue (G, designating glycine) is residue 1 and the rightmost residue (S, designating serine) is residue 24. In this text, standard one-letter abbreviations and/or standard three-letter abbreviations may be used for residues (amino acids): for example, histidine may be abbreviated as H or His. When referring to modifications made to this peptide sequence or to another peptide sequence, the number of a residue given refers to the original peptide sequence. For example, replacement of residue 4 of this peptide sequence by proline (P) refers to replacement of valine (V) residue 4 with proline (P). This can be abbreviated as V4P: with reference to the cloacaenodin peptide sequence that is modified, this can be abbreviated as cloacaenodin V4P. For example, insertion of alanine (A) after residue 4 in this peptide sequence refers to insertion of alanine (A) after valine (V) residue 4 and before aspartate (D) residue 5, that is, to insertion of alanine (A) between valine (V) residue 4 and aspartate (D) residue 5. For example, removal of residues 16 and 17 from this peptide sequence refers to removal of leucine (L) residue 16 and removal of proline (P) residue 17, so that glycine (G) residue 15 and glycine (G) residue 18 become adjacent to and bonded to each other.

In this text, standard one-letter abbreviations may be used for nucleotides; for example, cytosine may be abbreviated as C or c.

In an embodiment of the invention, the cloacaenodin-class lasso peptide is not cloacaenodin of the peptide sequence GHSVDRIPEYFGPPGLPGPVLFYS [SEQ ID NO. 1], not cloacaenodin-2 of the peptide sequence GHSVDRIPEYFGPPGLPSPVLFYS [SEQ ID NO. 105], and not cloacaenodin-3 of the peptide sequence GHSVDRIPEYFGPPGGPILFYS [SEQ ID NO. 106].

The ring of the cloacaenodin-class lasso peptide can be formed of nine (9) residues:

for example, the ring can be formed of the subsequence GHSVDRIPE [SEQ ID NO. 9] (of the cloacaenodin-class lasso peptide). The ring of a cloacaenodin-class lasso peptide can be formed of ten (10) residues: for example, the ring can be formed of the subsequence GHSVADRIPE [SEQ ID NO. 7]. The loop region of the cloacaenodin-class lasso peptide can be formed of thirteen (13) residues: for example, the loop region can be formed of the subsequence YFGPPGLPGPVLF [SEQ ID NO. 115]. Alternatively, the loop region can be formed of twelve (12) residues or eleven (11) residues. The tail region of the cloacaenodin-class lasso peptide can be formed of two (2) residues: for example, the tail region can be formed of the subsequence YS.

As indicated above, modifications to a cloacaenodin-class lasso peptide sequence GHSVDRIPEYFGPPGLPGPVLFYS [SEQ ID NO. 1] can be made. This peptide sequence can be constrained, so that residues 22 through 24 of the peptide sequence are not removed or replaced. This peptide sequence can be constrained, so that at most two (2) of residues 12 through 21 of the peptide sequence are removed or replaced. Residue 8 of the peptide sequence can be proline (P). Residue 8 of the peptide sequence can be replaced by alanine (A). Residue 4 of the peptide sequence can be replaced by proline (P). Residue 22 of the peptide sequence can be replaced by tryptophan (W). Residue 23 of the peptide sequence can be replaced by tryptophan (W). Residue 24 of the peptide sequence can be replaced by alanine (A). Residue 24 of the peptide sequence can be replaced by tyrosine (Y). Residue 24 of the peptide sequence can be replaced by threonine (T). Residue 24 of the peptide sequence can be replaced by cysteine (C). Residue 10 of the peptide sequence can be replaced by alanine (A). Alanine (A) can be inserted after residue 4 of the peptide sequence. Residue 18 of the peptide sequence can be serine (S). Residue 20 of the peptide sequence can be isoleucine (I). Residues 16 and 17 of the peptide sequence can be removed.

The cloacaenodin-class lasso peptide can be cloacaenodin of the peptide sequence GHSVDRIPEYFGPPGLPGPVLFYS [SEQ ID NO. 1]. The cloacaenodin-class lasso peptide can be cloacaenodin-2 of the peptide sequence GHSVDRIPEYFGPPGLPSPVLFYS [SEQ ID NO. 105]. The cloacaenodin-class lasso peptide can be cloacaenodin-3 of the peptide sequence GHSVDRIPEYFGPPGGPILFYS [SEQ ID NO. 106]. The cloacaenodin-class lasso peptide can be of a peptide sequence GHSVADRIPEYFGPPGLPGPVLFYS [SEQ ID NO. 67], GHSVDRIAEYFGPPGLPGPVLFYS [SEQ ID NO. 109],

[SEQ ID NO. 110]
GHSPDRIPEYFGPPGLPGPVLFYS,
[SEQ ID NO. 111]
GHSVDRIPEYFGPPGLPGPVLWYS,
[SEQ ID NO. 112]
GHSVDRIPEYFGPPGLPGPVLFWS,
[SEQ ID NO. 113]
GHSVDRIPEYFGPPGLPGPVLFYA,
[SEQ ID NO. 114]
GHSVDRIPEYFGPPGLPGPVLFYY,
[SEQ ID NO. 116]
GHSVDRIPEYFGPPGLPGPVLFYT,
[SEQ ID NO. 117]
GHSVDRIPEYFGPPGLPGPVLFYC
or
[SEQ ID NO. 118]
GHSVDRIPEAFGPPGLPGPVLFYS.

The peptide sequence of the cloacaenodin-class lasso peptide can be at least 70%, at least 75%, at least 80%, at least 85%, at least 90%, or at least 95% homologous to

[[SEQ ID NO. 1]
GHSVDRIPEYFGPPGLPGPVLFYS.

The cloacaenodin-class lasso peptide can be threaded. That is, the cloacaenodin-class lasso peptide can have a threaded structure. The tail region can be threaded through the ring. The tail region can be passed through the empty center portion of the ring. The residue of the tail region that is bonded to the loop region and the residue of the loop region that is bonded to the tail region can be on opposite sides of the ring. For example, with the ring approximated by a plane passing through the residues forming the ring, a residue “y” of the tail region can be on one side of that plane, and the residue “x” of the loop region to which residue “y” is bonded can be on the opposite side of that plane. For example, the tail region of a subsequence YS can be threaded through the ring of a subsequence GHSVDRIPE [SEQ ID NO. 9]. The residue Y (tyrosine) of the tail region can be on one side of the ring, and the residue F (phenylalanine) (to which the residue Y (tyrosine) of the tail region is bonded) of the loop region can be on the opposite side of the ring. For example, the ring can be formed of nine (9) residues, and the cloacaenodin-class lasso peptide can include a threaded structure.

A pharmaceutical composition can include the cloacaenodin-class lasso peptide. The pharmaceutical composition can further include a pharmaceutically acceptable carrier or diluent. The pharmaceutical composition can be in a dosage form, such as an injectable liquid, a capsule, a tablet, a pill, a suppository, a powder, a time-release capsule, a time-release tablet, a time-release pill, a time-release suppository, a cream, an ointment, a gel, or an impregnated wound dressing. For example, the concentration of the cloacaenodin-class lasso peptide in the pharmaceutical composition can be less than 10 μM. For example, the cloacaenodin-class lasso peptide can be provided within a pharmaceutical composition.

The microorganism can be a gammaproteobacterium, for example, a gram-negative gammaproteobacterium. The microorganism can be of order Enterobacterales, of family Enterobacteriaceae, of genus Enterobacter, or of genus Kluyvera. For example, the microorganism can be a species of Enterobacter, such as Enterobacter amnigenus, Enterobacter asburiae, Enterobacter mori, Enterobacter nimipressuralis, Enterobacter cloacae, Enterobacter hormaechei, Enterobacter kobei, Enterobacter ludwigii, or Enterobacter xiangfangensis. For example, the microorganism can be a species of Kluyvera, such as Kluyvera ascorbata. The microorganism can be resistant to an antibiotic, resistant to a broad-spectrum antibiotic, resistant to an antibiotic of last resort, resistant to a beta-lactam antibiotic, or resistant to a carbapenem.

The microorganism can be exposed to the cloacaenodin-class lasso peptide in vitro. For example, such in vitro exposure of a microorganism to the cloacaenodin-class lasso peptide can be used to determine whether and at what concentration the cloacaenodin-class lasso peptide inhibits growth of the microorganism, for example, to determine the minimal inhibitory concentration (MIC). For example, the cloacaenodin-class lasso peptide can exhibit an MIC against the microorganism of 15 μM or less, 10 μM or less, 8 μM or less, 4 μM or less, 2 μM or less, 1 μM or less, 0.5 μM or less, or 0.25 μM or less.

The microorganism can be exposed to the cloacaenodin-class lasso peptide within or on a patient.

In a method according to the invention, a patient infected with the microorganism is treated, including by administering the cloacaenodin-class lasso peptide to the patient, so that the patient is treated. In a method according to the invention, a patient can be treated (for example, treated prophylactically) to prevent infection of the patient by the microorganism, including by administering the cloacaenodin-class lasso peptide to the patient, so that the infection of the patient by the microorganism is prevented. For example, the cloacaenodin-class lasso peptide can be administered to the patient intravenously, intraperitoneally, intramuscularly, orally, by nasal insufflation, by inhalation, topically, vaginally, urethrally, or rectally. For example, the patient can be a human, can be an animal, can be a mammal, can be a nonhuman animal, or can be a nonhuman mammal. For example, the patient can be a plant.

The cloacaenodin-class lasso peptide can be for use as a medicament. The cloacaenodin-class lasso peptide can be for use in the treatment of an infection with a microorganism. The cloacaenodin-class lasso peptide can be for use in the prevention of an infection with a microorganism. The cloacaenodin-class lasso peptide can be used in the manufacture of a medicament for the treatment of an infection with a microorganism. The cloacaenodin-class lasso peptide can be used in the manufacture of a medicament for the prevention of an infection with a microorganism.

In a method according to the invention, a cloacaenodin-class lasso peptide is produced, including by refactoring the precursor, protease, cyclase, and exporter genes for cloacaenodin into a plasmid in vitro, transforming the plasmid into cells in vitro, growing the cells in vitro, and inducing expression of the cloacaenodin-class lasso peptide by the cells in vitro. For example, the cells can be Escherichia coli (E. coli). A supernatant can be separated from the cells, for example, by centrifugation. The cloacaenodin-class lasso peptide (for example, a purified cloacaenodin-class lasso peptide) can be obtained from the supernatant, for example, by extraction, chromatography, reversed-phase (RP) chromatography, high-performance liquid chromatography (HPLC), and/or RP-HPLC. The cloacaenodin-class lasso peptide can be frozen at 0° C. or less, −10° C. or less, −20° C. or less, −40° C. or less, −60° C. or less, or −80° C. or less within 15 minutes, within 30 minutes, within 45 minutes, or within 60 minutes of obtaining the cloacaenodin-class lasso peptide from the supernatant. The cloacaenodin-class lasso peptide can be cloacaenodin of the peptide sequence GHSVDRIPEYFGPPGLPGPVLFYS [SEQ ID NO. 1].

Site-directed mutagenesis can be used to modify the plasmid. The cloacaenodin-class lasso peptide (which can be purified) can include the peptide sequence GHSVDRIPEYFGPPGLPGPVLFYS [SEQ ID NO. 1]. Zero (0), one (1), or two (2) of residues 2 through 8 of the peptide sequence can be removed or replaced with another residue. Zero (0) or one (1) residue can be inserted after one of residues 1 through 8. Zero (0), one (1), two (2), three (3), four (4), or five (5) of residues 12 through 24 of the peptide sequence can be removed or replaced. Zero (0), one (1), two (2), three (3), or four (4) residues can be inserted after at least one of residues 11 through 21. For example, the cloacaenodin-class lasso peptide can be cloacaenodin-2 of a peptide sequence GHSVDRIPEYFGPPGLPSPVLFYS [SEQ ID NO. 105] or cloacaenodin-3 of a peptide sequence GHSVDRIPEYFGPPGGPILFYS [SEQ ID NO. 106]. For example, the cloacaenodin-class lasso peptide can be of the peptide sequence

[SEQ ID NO. 67]
GHSVADRIPEYFGPPGLPGPVLFYS,
[SEQ ID NO. 109]
GHSVDRIAEYFGPPGLPGPVLFYS
[SEQ ID NO. 110]
GHSPDRIPEYFGPPGLPGPVLFYS,
[SEQ ID NO. 111]
GHSVDRIPEYFGPPGLPGPVLWYS,
[SEQ ID NO. 112]
GHSVDRIPEYFGPPGLPGPVLFWS,
[SEQ ID NO. 113]
GHSVDRIPEYFGPPGLPGPVLFYA,
[SEQ ID NO. 114]
GHSVDRIPEYFGPPGLPGPVLFYY,
[SEQ ID NO. 116]
GHSVDRIPEYFGPPGLPGPVLFYT,
[SEQ ID NO. 117]
GHSVDRIPEYFGPPGLPGPVLFYC,
or
[SEQ ID NO. 118]
GHSVDRIPEAFGPPGLPGPVLFYS.

### BRIEF DESCRIPTION OF THE DRAWINGS

FIG. 1A. A new lasso peptide is identified from Enterobacter species through genome mining. The native biosynthetic gene cluster (BGC) is an organization of lasso peptide genes in proteobacteria. It contains the precursor gene, cloA: the leader peptidase gene, cloB: the lasso peptide cyclase gene, cloC; and an adenosine triphosphate (ATP)-binding cassette transporter gene for export of the mature lasso peptide, cloD. To facilitate heterologous expression, the BGC was codon-optimized and refactored into a pQE-80 vector with the cloA gene under the control of an isopropyl-β-D-thiogalactopyranoside (IPTG)-inducible T5 promoter, while the rest of the cluster is under the control of the constitutive p_{mcjBCD} promoter.

FIG. 1B. The sequence of cloacaenodin and other known RNA polymerase (RNAP)-inhibiting lasso peptides. Cloacaenodin contains a 9-membered (9-residue) ring (lighter font) and a C-terminal serine, which differs from the other lasso peptides, which contain an 8-membered (8-residue) ring and a C-terminal glycine. Cloacaenodin contains the relatively well-conserved tyrosine (Y) residue directly after the ring, and the conserved penultimate tyrosine (Y) residue (underlined).

FIG. 2A. Cloacaenodin can be biosynthesized heterologously in E. coli. High-performance liquid chromatography (HPLC) chromatogram of the M9 supernatant extract from E. coli heterologous expression of cloacaenodin (darker trace) and purified cloacaenodin (lighter trace).

FIG. 2B. Mass spectrum of purified cloacaenodin displaying the +4, +3, and +2 charge states. A close-up of the +3 charge state isotopic distribution is shown. The expected monoisotopic +3 m/z is 861.77.

FIG. 3A. NMR structure of cloacaenodin. Representative cloacaenodin structure is shown. The steric locks Phe22 and Tyr23 are indicated.

FIG. 3B. The twenty (20) lowest energy structures are overlaid. These model structures have been deposited to the Protein Data Bank under PDB code 8DYN.

FIG. 4. Threaded cloacaenodin is resistant to proteolysis while the unthreaded conformer is proteolyzed into the major products shown. Loop region and tail region residues are darker and ring residues are lighter. Phe22 and Tyr23 are enlarged to denote their role as the steric lock residues for cloacaenodin. The liquid chromatography-mass spectrometry (LC-MS) data underlying this schematic are shown in FIGS. 17-20.

FIG. 5. An alignment of the A proteins from cloacaenodin-like BGCs, found from a BlastP search on the CloA sequence. The region with the least degree of similarity is the N-terminal portion of the leader, which is consistent with most of the recognition by the B protein being at the C-terminal end of the leader and the beginning of the core peptide. All of the leader peptides consist of 32-34 amino acids with a core peptide of 24 amino acids (with the exception of the Citrobacter core of 22 amino acids).

FIG. 6. LC-MS trace of supernatant extract for expression of cloacaenodin. Top: Total ion current (TIC) chromatogram of supernatant extract. Bottom: Extracted ion current (EIC) chromatogram of supernatant extracted for expected +3 and +2 mass-charge states. Extraction was for the predicted monoisotopic charge-states of a +3 m/z of 861.7725 and a +2 m/z of 1292.1552.

FIG. 7A. The threaded cloacaenodin stays intact when treated with carboxypeptidase, while the unthreaded cloacaenodin, which can be generated by heating at 95° C., is susceptible to proteolysis by carboxypeptidase. C-terminally truncated fragments of cloacaenodin can be detected only when the unthreaded peptide is treated. TIC chromatograms of cloacaenodin under the conditions indicated.

FIG. 7B. Table of observed masses and proposed sequences. For each value, the charge state is shown in parentheses.

FIG. 8A. Cartoon depicting the product of threaded cloacaenodin upon treatment with carboxypeptidase. The threaded peptide is not proteolyzed by carboxypeptidase as its C-terminus is protected by the ring.

FIG. 8B. Cartoon depicting the products of unthreaded cloacaenodin upon treatment with carboxypeptidase. The unthreaded cloacaenodin can be truncated at the C-terminus by 6, 9, or 10 amino acids. Steric lock residues are shown by larger circles.

FIG. 9. An 18 μM sample of cloacaenodin stays >80% threaded after incubating at 37° C. over a period of 3 days, showing that the peptide's bioactivity would not be completely inactivated by unthreading at this timescale.

FIG. 10A. Both threaded and unthreaded cloacaenodin yield similar MS/MS fragmentation patterns, but the threaded cloacaenodin is more resistant to fragmentation due to its interlocked structure. Various peaks were only observable for the unthreaded species. Tandem mass spectrometry (MS/MS) data of threaded cloacaenodin are shown.

FIG. 10B. MS/MS of unthreaded cloacaenodin.

FIG. 10C. Fragment ions observed. For each value, the charge state is shown in parentheses.

FIG. 11. Cloacaenodin remained threaded throughout NMR acquisition by keeping it at 4° C., as shown by the single peak on LC-MS before and after acquisition.

FIG. 12. One-dimensional (ID) nuclear magnetic resonance (NMR) spectrum taken before (top) and after (bottom) two-dimensional (2D) NMR acquisition ensured the sample remained stable. The ID NMR spectrum stayed consistent.

FIG. 13. Total correlation spectroscopy (TOCSY) results for cloacaenodin at a mixing time of 80 ms.

FIG. 14. Nuclear Overhauser effect spectroscopy (NOESY) results for cloacaenodin at a mixing time of 150 ms.

FIG. 15. NOESY results for cloacaenodin at a mixing time of 300 ms.

FIG. 16. NMR structures of cloacaenodin and other gram-negative-targeting lasso peptides. The lasso peptides share a large loop, short tail structure.

FIG. 17A. Threaded cloacaenodin is resistant to trypsin digestion while unthreaded cloacaenodin can be cleaved by trypsin. The cartoon depicts major products of threaded (top) and unthreaded (bottom) cloacaenodin upon trypsin treatment. Steric lock residues are shown larger. The arginine (R) residue in the ring is preferentially susceptible to trypsin digestion.

FIG. 17B. LC-MS data of trypsin proteolysis experiment on threaded and unthreaded cloacaenodin, with a table of observed masses and proposed sequences. The charge state for each value is shown in parentheses.

FIG. 18A. Threaded cloacaenodin is resistant to chymotrypsin digestion while unthreaded cloacaenodin can be cleaved by chymotrypsin. The cartoon depicts that threaded cloacaenodin is not affected by chymotrypsin treatment. Residues that are preferentially susceptible to chymotrypsin digestion are phenylalanine (F) and tyrosine (Y).

FIG. 18B. The cartoon depicts major products of unthreaded cloacaenodin upon chymotrypsin treatment. Residues that are preferentially susceptible to chymotrypsin digestion are phenylalanine (F) and tyrosine (Y).

FIG. 18C. LC-MS data of chymotrypsin proteolysis experiment on threaded and unthreaded cloacaenodin.

FIG. 18D. Table of observed masses and proposed sequences from LC-MS data of chymotrypsin proteolysis experiment on threaded and unthreaded cloacaenodin. For each value, the charge state is shown in parentheses.

FIG. 19A. Threaded cloacaenodin is resistant to thermolysin digestion while unthreaded cloacaenodin can be cleaved by thermolysin. The cartoon depicts that threaded cloacaenodin is not affected by thermolysin treatment. Residues that are preferentially susceptible to thermolysin digestion are the residue 3^{rd} from the tail end (phenylalanine (F)), the residue 4^{th} from the tail end (leucine (L)), the residue 5^{th} from the tail end (valine (V)), the phenylalanine (F) in the loop, and the valine (V) in the ring. The tyrosine (Y) 2^{nd} from the tail end (shown larger) acts as a steric lock residue.

FIG. 19B. The cartoon depicts major products of unthreaded cloacaenodin upon thermolysin treatment. Residues that are preferentially susceptible to thermolysin digestion are the residue 3^{rd} from the tail end (phenylalanine (F)), the residue 4^{th} from the tail end (leucine (L)), the residue 5^{th} from the tail end (valine (V)), the phenylalanine (F) in the loop, and the valine (V) in the ring.

FIG. 19C. LC-MS data of thermolysin proteolysis experiment on threaded and unthreaded cloacaenodin.

FIG. 19D. Table of observed masses and proposed sequences from LC-MS data of thermolysin proteolysis experiment on threaded and unthreaded cloacaenodin. For each value, the charge state is shown in parentheses.

FIG. 20A. Threaded cloacaenodin is resistant to elastase digestion while unthreaded cloacaenodin can be cleaved by elastase. The cartoon depicts that threaded cloacaenodin is not affected by elastase treatment. Residues that are preferentially susceptible to elastase digestion are the serine (S) in the ring and the leucine (L), valine (V), glycine (G), and isoleucine (I). The tyrosine (Y) 2^{nd} from the tail end and the phenylalanine (F) 3^{rd} from the tail end (shown larger) act as steric lock residues.

FIG. 20B. The cartoon depicts major products of unthreaded cloacaenodin upon elastase treatment. Residues that are preferentially susceptible to elastase digestion are the serine (S) in the ring and the leucine (L), valine (V), glycine (G), and isoleucine (I).

FIG. 20C. LC-MS data of elastase proteolysis experiment on threaded and unthreaded cloacaenodin.

FIG. 20D. Table of observed masses and proposed sequences from LC-MS data of elastase proteolysis experiment on threaded and unthreaded cloacaenodin. For each value, the charge state is shown in parentheses.

FIG. 21. When the cloD gene is deleted from the cloacaenodin expression plasmid for Escherichia coli (E. coli), E. coli cells can no longer grow in media that induces expression of cloacaenodin. Cloacaenodin Y10A is not toxic intracellularly to E. coli. On left plate: Grown on media with glucose to repress expression of pQE-80 derived plasmid, E. coli cells containing expression plasmid for wild-type cloacaenodin without CloD (left) and E. coli cells containing expression plasmid cloacaenodin Y10A without CloD (right). On right plate: Grown on media with IPTG to induce expression of pQE-80 derived plasmid, E. coli cells containing expression plasmid for wild-type cloacaenodin without CloD (left) and E. coli cells containing expression plasmid cloacaenodin Y10A without CloD (right).

FIG. 22A. Spot-on-lawn assays in M63 agar of cloacaenodin against commercially acquired Enterobacter species. 1.) Enterobacter cloacae (E. cloacae) ATCC 13047; 2.) Enterobacter nimipressuralis DSM 18955; 3.) Enterobacter kobei BAA-260; 4.) Enterobacter hormaechei ATCC 700323; 5.) Enterobacter asburiae DSM 17506; 6.) Enterobacter mori DSM 26271; 7.) Enterobacter amnigenus (E. amnigenus) ATCC 33072.

FIG. 22B. Selected spot-on-lawn assays in M63 agar of cloacaenodin against clinical isolates of Enterobacter species (full list of tested strains in Table 7). These strains consistently showed low micromolar MICs in the assay. UCI35, MGH243, and UCI193 are classified as carbapenem resistant.

FIG. 23. At a concentration of 15 μM, only threaded cloacaenodin has activity against Enterobacter cloacae. Unthreaded cloacaenodin has no activity at this concentration against Enterobacter cloacae.

FIG. 24A. Liquid inhibition assays in M63 media of cloacaenodin activity against Enterobacter cloacae and Enterobacter amnigenus. Optical density at 600 nm (OD₆₀₀) data after 8 hours are shown.

FIG. 24B. Liquid inhibition assays in M63 media of cloacaenodin activity against Enterobacter cloacae and Enterobacter amnigenus. OD₆₀₀ data after 16 hours are shown.

FIG. 25A. When treated with sub-MIC values of cloacaenodin, Enterobacter cloacae exhibits a filamentation phenotype. Untreated Enterobacter cloacae, grown for 16 hours in M63 media.

FIG. 25B. When treated with sub-MIC values of cloacaenodin, Enterobacter cloacae exhibits a filamentation phenotype. Enterobacter cloacae treated with 230 nM of cloacaenodin and grown for 16 hours in M63 media.

FIG. 26. Changing of the lower steric lock (Tyrosine (Y) 23) to a bulkier tryptophan (Trp, W) residue leads to only threaded cloacaenodin Y23W observed in the supernatant extract. Top: EIC of supernatant extract of cloacaenodin Y23W, extracted for the expected monoisotopic +2 and +3 charge states (1303.6632 Da and 869.4445 Da, respectively). Bottom: EIC of cloacaenodin Y23W in an HPLC fraction and heated at 95° C. This was done to verify that the peak at around 11.5 minutes in the supernatant extract (top trace) was threaded. The peak representing unthreaded cloacaenodin Y23W (bottom trace) was not observed in the supernatant extract (top trace).

FIG. 27A. After expression and purification of the peptide, the major cloacaenodin S24G product was unthreaded. This is supported by the later retention time compared to wild-type cloacaenodin, a lack of shift in retention time upon heating, and susceptibility to carboxypeptidase. The dehydrated product 5 is likely due to aspartate (Asp, D) dehydration in the ring.

FIG. 27B. Table summarizing data from FIG. 27A.

FIG. 28. Bioactivity of cloacaenodin variants against Enterobacter amnigenus. Peptides were tested at a concentration of 10 μM. Based on inhibition diameter, all variants appear to have similar or reduced activity compared to wild-type (WT) cloacaenodin.

FIG. 29. Despite being mostly unthreaded after purification, cloacaenodin S24G exerts intracellular toxicity in Escherichia coli XL-1 blue cells when expression is induced with IPTG in a construct lacking cloD. This result indicates that at least some threaded peptide exists intracellularly to exert antimicrobial activity.

FIG. 30. A variant of cloacaenodin with a 10-membered (10-residue) ring (cloacaenodin-10) can be biosynthesized and is detected in the supernatant. Top: EIC of cloacaenodin-10 in the supernatant extract. Middle: EIC of cloacaenodin-10 in supernatant extract after heating. The retention time does not shift. Bottom: EIC of cloacaenodin-10 in supernatant extract after treatment with carboxypeptidase. The peak detected at 12.1 minutes is no longer detected, suggesting that the cloacaenodin-10 was susceptible to degradation by carboxypeptidase. Extraction was for the predicted monoisotopic charge states of a +3 m/z of 885.4516 and a +2 m/z of 1327.6737.

FIG. 31. Cartoon depicting proposed biosynthesis of cloacaenodin with a 10-membered (10-residue) ring. Without being bound by theory, this lasso peptide variant may be made threaded and then unthread upon secretion into the supernatant and during purification.

FIG. 32. TIC (top trace) and EIC (bottom trace) chromatograms of a cloacaenodin P8A variant from supernatant extract. Extraction was for the predicted monoisotopic charge states of a +3 m/z of 853.1007 and a +2 m/z of 1279.1473.

FIG. 33. TIC (top trace) and EIC (bottom trace) chromatograms of a cloacaenodin V4P variant from supernatant extract. Extraction was for the predicted monoisotopic charge states of a +3 m/z of 861.1007 and a +2 m/z of 1291.1473.

FIG. 34. Purified cloacaenodin Y10A has reduced bioactivity against Enterobacter amnigenus. Spot 1 is 120 μM, and two-fold serial dilutions were spotted clockwise. Similar data for the wild-type peptide are shown in FIG. 22A.

FIG. 35. Peptide sequences of cloacaenodin, cloacaenodin-2, and cloacaenodin-3 precursors.

FIG. 36. Cloacaenodin-2 can be heterologously expressed in Escherichia coli and purified from the supernatant. The HPLC traces are shown with arrows indicating which peak was collected during the run. The top trace shows results from the first round of HPLC purification: the bottom trace shows results from the second round of HPLC purification.

FIG. 37A. LC-MS analysis of purified cloacaenodin-2. Top: Total ion current (TIC) chromatogram of purified cloacaenodin-2. Bottom: UV absorbance measured at 215 nm of purified cloacaenodin-2.

FIG. 37B. Mass spectrum of cloacaenodin-2 indicating the +3 and +2 charge-states. Predicted monoisotopic +3 charge-state is 871.7761. Observed monoisotopic +3 charge-state is 871.7596. The figure displays the highest intensity peaks from the mass spectrum.

FIG. 38. Cloacaenodin-3 can be heterologously expressed in Escherichia coli and purified from the supernatant. The HPLC traces are shown with arrows indicating which peak was collected during the run. The top trace shows results from the first round of HPLC purification: the bottom trace shows results from the second round of HPLC purification.

FIG. 39A. LC-MS analysis of purified cloacaenodin-3. Top: TIC chromatogram of purified cloacaenodin-3. A contaminant elutes immediately before cloacaenodin-3, but based on the UV-215 nm spectrum (see spectrum at bottom), this contaminant is present at minor amounts compared to the peptide (˜90% purity). Bottom: UV absorbance measured at 215 nm for purified cloacaenodin-3.

FIG. 39B. Mass spectrum of cloacaenodin-3 indicating the +3 and +2 charge-states. Predicted monoisotopic +3 charge-state is 796.3988. Observed monoisotopic +3 charge-state is 796.3767. The figure displays the highest intensity peaks from the mass spectrum.

FIG. 40. Summary of biosynthetic strategy to produce cloacaenodin-2 and cloacaenodin-3. In the cloacaenodin expression plasmid, the DNA encoding the core peptide was mutated via site-directed mutagenesis, so that it encoded cloacaenodin-2 and cloacaenodin-3 core peptide sequences. The sequences encoding the cloacaenodin leader peptide, CloB, CloC, and CloD, were left unchanged. Cloacaenodin-2 and cloacaenodin-3 were successfully produced in Escherichia coli using these expression plasmids and HPLC-purified from the supernatant extract. Cloacaenodin-3 has a predicted two (2) aa (amino acid) shorter loop, indicating tolerance of the cloacaenodin biosynthetic machinery for this reduced loop size.

FIG. 41. Spot-on-lawn assay of cloacaenodin, cloacaenodin-2, and cloacaenodin-3 against E. amnigenus in M63 agar. Spot 1 is cloacaenodin spotted at a concentration of 120 μM. Spot 2 is cloacaenodin-2 spotted at a concentration of 120 u M. Spot 3 is cloacaenodin-3 spotted at a concentration of 63 μM. 4 is pure water.

FIG. 42. Spot-on-lawn assays of tested clinical isolates treated with cloacaenodin in M63 agar. From the initial concentration shown to the left of the curved arrow and in the direction of the curved arrow, two-fold serial dilutions of cloacaenodin were spotted. The straight arrows point to the last visible spot on the plate which corresponds to the recorded minimal inhibitory concentration (MIC). Spot 8 is pure water in all plates. AR Bank #0132, AR Bank #0136, AR Bank #0144, and AR Bank #0154 are from the CRE panel. AR Bank #0001 and AR Bank #0002 are from the BIT panel.

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

Bacterial infections continue to be a scourge on humanity. Examples of problematic pathogenic bacteria are 1) bacteria that cause infections in hospital settings, i.e., nosocomial infections, and 2) bacteria that have acquired antimicrobial resistance, including antibiotic resistance. The ESKAPE pathogens are species of bacteria that are of clinical concern for their ability to evade the mechanisms of current antibiotics. Without new drugs and treatments, these resistant species present a threat to humanity, causing an unnecessary loss of life to infections once treatable. The last “E” in “ESKAPE” represents species of Enterobacter, which is a genus of Gram-negative γ-proteobacteria.² While Enterobacter species commonly reside commensally in the human and animal GI tract and in the environment on decaying matter, in soil, and in sewage, certain species such as Enterobacter cloacae have been the causative agents of many nosocomial outbreaks.²⁻⁴ Within this Enterobacter genus is the Enterobacter cloacae complex (ECC), which includes closely related species that are isolated as clinical specimens, for example Enterobacter cloacae and Enterobacter hormaechei.⁴⁻⁵ A number of these pathogens have natural resistance to β-lactam antibiotics and possess various carbapenemase genes.³ Colistin resistance has also been found in Enterobacter infections.⁶⁻¹² With the rise of multidrug resistance in Enterobacter bacteria, for example, with the emergence of Enterobacter strains displaying resistance to last-resort antibiotics such as carbapenems, new treatments are needed.³

Lasso peptides are named after their unusual threaded shape, resembling a slipknot. Lasso peptides may exhibit antimicrobial activity by several different mechanisms.³⁻²⁴ That is, lasso peptides can have a lariat-knot shape and narrow-spectrum antimicrobial activity against clinically relevant pathogens.^{25,60} Lasso peptides are ribosomally synthesized and post-translationally modified peptides. That is, lasso peptides are biosynthesized from a ribosomal precursor peptide (known as A) via the action of two enzymes, a protease (known as B) and lasso cyclase (known as C).²⁵⁻²⁹ The compact, threaded structure of lasso peptides shields portions of the amide backbone, which can render a lasso peptide protease resistant. Genomic sequencing can be used to find and predict lasso peptides and other ribosomally synthesized and post-translationally modified peptides (RiPPs) that may not be detected from cultivation of the native species in the lab. This can be used in the discovery of potential new drug compounds.³⁰⁻³⁵

A lasso peptide can display focused spectra of activity: this can provide a route to target specific pathogens without disturbing commensal bacteria.³⁶⁻³⁸ From past investigations with lasso peptides such as ubonodin⁶, citrocin, klebsidin⁹, microcin J25 (MccJ25)¹³, and capistruin¹⁴, it was noticed that these compounds can target strains that are phylogenetically similar to the producer, potentially serving as a mechanism for competition in microbial communities.³⁹ Without being bound by theory, lasso peptide biosynthetic gene clusters (BGCs) found in pathogen-related species may have antimicrobial activity against clinically relevant pathogens (a guilt-by-association approach), presenting a way to screen and prioritize genome mining hits. For example, a genome mining approach can be focused on organisms that are pathogen adjacent towards discovering lasso peptides with antimicrobial activity against a pathogen of interest.^{29,16,19}

Genome mining was used to identify a lasso peptide, now named cloacaenodin, which has potent antimicrobial activity, from the Enterobacter cloacae complex (ECC). The solution structure of this lasso peptide cloacaenodin was determined (solved) by 2D NMR. That is, by using NMR and mass spectrometric analysis this lasso peptide cloacaenodin was shown to include a threaded lasso fold which imparts proteolytic resistance that unthreaded peptides lack. Most peptides can be destroyed by general proteases that reside in the body; however, cloacaenodin, because of its unique shape, is resistant to proteolysis under conditions under which unthreaded linear, branched, and/or cyclic peptides are proteolyzed. That is, this lasso peptide cloacaenodin resists cleavage by proteases, including common proteases, thus having an advantage over linear peptides. This lasso peptide cloacaenodin has enhanced stability and may have enhanced stability in a clinical setting.

The breadth of the spectrum of antimicrobial activity of this lasso peptide, cloacaenodin, and its potential for treating nosocomial infections were assessed. For example, cloacaenodin showed potent and selective activity against multiple clinically relevant strains from the ECC. That is, an embodiment of the invention is the lasso peptide cloacaenodin, which has antimicrobial activity against species and strains of the bacterial genus Enterobacter and other genera, including nearby genera. Cloacaenodin was tested against a set of Enterobacter species and strains, and species and strains against which cloacaenodin has activity (i.e., the ability to inhibit growth or kill such species and strains) were identified. The lasso peptide cloacaenodin has potent activity against multiple species and strains of bacteria, including Enterobacter species and strains (such as members of the pathogenic Enterobacter cloacae complex (ECC)). That is, cloacaenodin can inhibit the growth of and/or kill such bacterial species and strains. Furthermore, cloacaenodin can do so without disruption of the native human microbiome, which includes beneficial bacteria. As discussed herein, cloacaenodin was shown to have activity against clinical strains (isolated from hospital patients) of Enterobacter that are resistant to other antibiotics (e.g., last-resort carbapenem antibiotics). For example, cloacaenodin has selective, low micromolar (minimal inhibitory concentration), antimicrobial activity against species related to the Enterobacter cloacae complex, including species implicated in nosocomial infections, and against clinical isolates of carbapenem-resistant Enterobacterales. Variants of this lasso peptide cloacaenodin were made that also have antimicrobial activity.

The lasso peptide cloacaenodin and its derivatives and variants can be used as pharmaceutical drugs that are an antimicrobial agents, for example, for the treatment of bacterial infections. The lasso peptide cloacaenodin acts as a narrow-spectrum antibiotic, which means that it is active only against certain species and strains of bacteria. This provides the advantage of being able to use cloacaenodin to target bacterial species and strains intended to be inhibited or killed, while leaving beneficial bacteria unharmed. This is in contrast with broad-spectrum antibiotics, which may inhibit or kill a broad range of species and strains of bacteria, including desirable bacteria. The lasso peptide cloacaenodin and its derivatives and variants can inhibit and kill bacterial species and strains that have evolved resistance to other antibiotics. The lasso peptide cloacaenodin and its derivatives and variants can be used alone or in combination with other antibiotics in therapy, for example, to treat a bacterial infection.

As discussed herein, recombinant DNA technology was used to express the lasso peptide cloacaenodin heterologously in E. coli. The cloacaenodin was purified via HPLC and its purity was confirmed. Structure-function analysis was carried out via mutagenesis of this lasso peptide. The mutagenesis experiments indicate aspects of the stability of this lasso peptide. That is, stability and structure-activity relationships of this lasso peptide cloacaenodin were studied via site-directed mutagenesis: site-directed mutagenesis was used to probe the importance of specific residues to the peptide's biosynthesis, stability, and bioactivity.

A lasso peptide may unthread over time at elevated temperatures when in solution. When a lasso peptide unthreads, it may lose its antimicrobial activity. This can be overcome by keeping the solution containing a lasso peptide cold at all times (for example, by keeping the solution frozen or on ice). A lasso peptide can also be kept long-term in lyophilized powder form without stability issues; that is, a lasso peptide can be maintained indefinitely substantially in its threaded configuration by having it in a lyophilized powder form. Cloacaenodin is shelf-stable indefinitely in its freeze-dried form.

### EXAMPLES

Genome Mining Reveals a New Lasso Peptide from Enterobacter Species

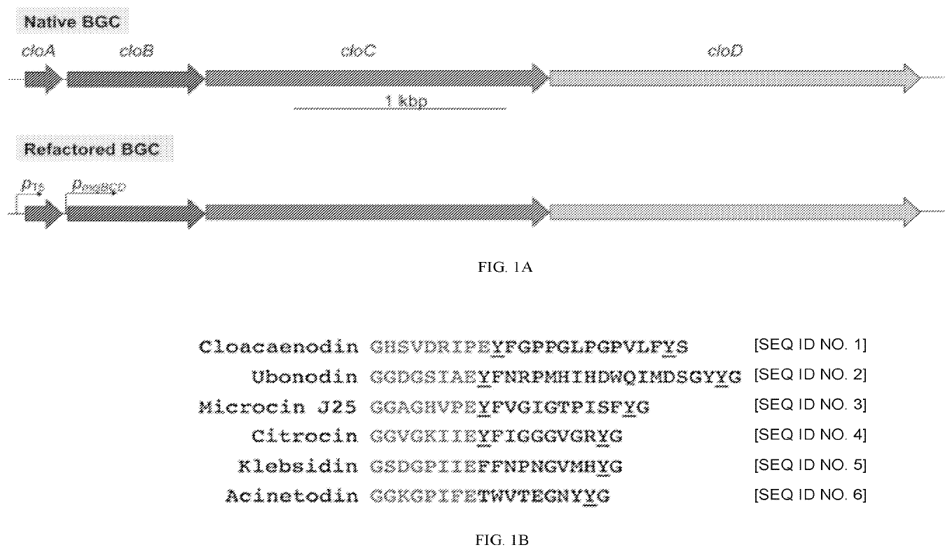

We employed a precursor-centric genome mining algorithm²⁶. Focus was on lasso peptides assumed to have a tyrosine (Tyr, Y) after the ring and a Tyr in the penultimate position. The corresponding Tyr residues in the lasso peptide MccJ25 make specific hydrogen binding contacts in the secondary channel of RNA (ribonucleic acid) polymerase (RNAP).⁴⁰ We identified a biosynthetic gene cluster (BGC) assumed to be a lasso peptide in Enterobacter hormaechei strain B3 (on contig NZ_LFHB01000019.1), a strain originally identified in long beans. We later identified identical BGCs in Enterobacter cloacae strain B2 (on contig NZ_JSWY01000033.1), a strain isolated from bitter gourd, and Enterobacter kobei strain 15727¹² (on contig NZ_JAKMKX010000007.1), a strain isolated from human sputum in China. We named this lasso peptide cloacaenodin (FIG. 1), because it originates from species of the Enterobacter cloacae complex and the Latin root “nodum” signifies knot.

A protein BLAST search on the CloA protein revealed 14 distinct protein accession numbers corresponding to cloacaenodin-like precursors from other strains of Salmonella, Escherichia coli, Citrobacter, and Enterobacter, including Enterobacter cloacae and Enterobacter hormaechei subsp. xiangfangensis (FIG. 5). We queried Enterobacter cloacae and Enterobacter hormaechei in an NCBI assembly search and found 616 total GenBank assemblies for Enterobacter cloacae and 3,754 total GenBank assemblies for Enterobacter hormaechei. Because we only observed the cloacaenodin BGC in a select few Enterobacter cloacae and Enterobacter hormaechei strains, this indicated to us that the cloacaenodin BGC is rare among the currently assembled genome sequences of these two species.

From the BLAST results, the presence of the B, C, and D genes (corresponding to the lasso peptide protease, cyclase, and exporter, respectively) downstream of the A gene (corresponding to the precursor) was manually confirmed. With the exception of the E. coli strains encoding a cloacaenodin-like precursor (FIG. 5) which appear to have their B and C genes disrupted, the amino acid sequences of the biosynthetic enzymes all have at least a 50% amino acid identity to CloB, CloC, and CloD, with the D proteins having the highest degree of identity (Tables 1-3).

TABLE 1
B proteins found near cloacaenodin-like A genes

| Species | Accession | Percent Identity |
|---|---|---|
| E. hormaechei LB3, E. cloacae B2, E. kobei 15727 | WP_046889164.1 | 100 |
| Enterobacter mori strain 08-091 | WP_215231253.1 | 91.63 |
| (plasmid pHS08-091) | | |
| Enterobacter hormaechei subsp. xiangfiangensis | MBT1945799.1 | 91.63 |
| strain 120070 | | |
| E. hormaechei subsp. xiangfangensis strain | HAV1851688.1 | 91.63 |
| ECC296 | | |
| Enterobacter ludwigii strain I140 | WP_104440573.1 & | 99.53 |
| | MBK1520793.1 | |
| Clinical sample of Salmonella enterica subsp. | EEJ6141376.1 | 79.53 |
| enterica serovar Urbana | | |
| Environmental sample of Salmonella enterica | EAM2342716.1 | 80.00 |
| E. cloacae strain N29 35 & | WP_194517084.1 | 79.07 (strain |
| strain MDR0071, e483 | (strain N29 35) & | N29 35) & |
| | WP_063955191.1 | 78.60 (strain |
| | (strain MDR0071, e483) | MDR0071, e483) |
| Clinical sample of Salmonella enterica subsp. | EEJ6141366.1 | 77.21 |
| enterica serovar Urbana | | |
| Environmental sample of E. coli | B disrupted | B disrupted |
| (strain PSU-1444) | | |
| E. coli E22 | B disrupted | B disrupted |
| Citrobacter sp. CtB7.12 | WP_054177363.1 | 53.95 |

TABLE 2
C proteins found near cloacaenodin-like A genes

| Species | Accession | Percent Identity |
|---|---|---|
| E. hormaechei LB3, E. cloacae 82, E. kobei 15727 | WP_046889163.1 | 100 |
| Enterobacter mori strain 08-091 | WP_215231254.1 | 86.19 |
| (plasmid pHS08-091) | | |
| Enterobacter hormaechei subsp. xiangfiangensis | MBT1945800.1 | 86.19 |
| strain 120070 | | |
| E. hormaechei subsp. xiangfangensis strain | HAV1851689.1 | 86.19 |
| ECC296 | | |
| Enterobacter ludwigii strain I140 | WP_185791751.1 & | 99.25 |
| | MBK1520792.1 | |
| Clinical sample of Salmonella enterica subsp. | EEJ6141377.1 | 72.05 |
| enterica serovar Urbana | | |
| Environmental sample of Salmonella enterica | EAM2342717.1 | 72.42 |
| E. cloacae strain N29 35 & | WP_194517083.1 | 72.98 (strain |
| strain MDR0071, e483 | (strain N29 35) & | N29 35) and |
| | WP_063955193.1 | 73.17 (strain |
| | (strain MDR0071, e483) | MDR0071, e483) |
| Clinical sample of Salmonella enterica subsp. | EEJ6141367.1 | 72.04 |
| enterica serovar Urbana | | |
| Environmental sample of E. coli | C disrupted | C disrupted |
| (strain PSU-1444) | | |
| E. coli E22 | C disrupted | C disrupted |
| Citrobacter sp. CtB7.12 | WP_054177364.1 | 52.91 |

TABLE 3
D proteins found near cloacaenodin-like A genes

| Species | Accession | Percent Identity |
|---|---|---|
| E. hormeechei LB3, E. cloacae 82, E. kobei 15727 | WP 072057270.1 | 100 |
| Enterobacter mori strain 08-091 | WP 215231255.1 | 94.64 |
| (plasmid pHHS08-091) | | |
| Enterobacter hormaechei subsp. xiangfiangensis | MBT1945801.1 | 94.29 |
| strain 120070 | | |
| E. hormaechei subsp. xiangfangensis strain | HAV1851690.1 | 94.29 |
| ECC296 | | |
| Enterobacter ludwigii strain I140 | WP_185791750.1 & | 99.83 |
| | MBK1520791.1 | |
| Clinical sample of Salmonella enterica subsp. | EEJ6141378.1 | 85.84 |
| enterica serovar Urbana | | |
| Environmental sample of Salmonella enterica | EAM2342718.1 | 85.15 |
| E. cloacae strain N29 35 & | WP_194517082.1 | 85.49 (strain |
| strain MDR0071, e483 | (strain N29 35) | MDR0071, |
| | (sequence cut short) & | e483) |
| | WP_080471846.1 (strain | |
| | MDR0071, e483) | |
| Clinical sample of Salmonella enterica subsp. | EEJ6141368.1 | 85.15 |
| enterica serovar Urbana | | |
| Environmental sample of E. coli | EFN8342409.1 | 84.80 |
| (strain PSU-1444) | | |
| E. coli E22 | WP_001299236.1 | 84.80 |
| Citrobacter sp. CtB7.12 | WP_054177365.1 | 70.93 |

More information on the genome mining procedure is in the section “Identification of Gene Cluster and Bioinformatic Search of Cloacaenodin-like Gene Clusters” under “Methods”, below.

Without being bound by theory, because of the producing species, this lasso peptide BGC was further considered, because it was hypothesized that this lasso peptide BGC may have activity against members of the Enterobacter cloacae complex (ECC). The gene cluster organization of ABCD (FIG. 1A) all in a single operon is observed in proteobacterial lasso peptide gene clusters¹⁴, with the gene clusters of citrocin and MccJ25 being exceptions in which the A genes are transcribed divergent1 from the BCD operon.^{17,41}.

Lasso peptides ubonodin¹⁶ and citrocin¹⁷ are inhibitors of ribonucleic acid (RNA) polymerase (RNAP), similar to the lasso peptides MccJ25, klebsidin, and acinetodin (FIG. 1B).^{19,40,42-44} Without being bound by theory, because of the presence of the conserved Tyr residues, it was hypothesized that cloacaenodin may also function by inhibiting RNAP. However, the C-terminal amino acid in cloacaenodin is serine (Ser, S), differing from the conserved glycine (Gly, G) seen in other RNAP-targeting lasso peptides (FIG. 1B). Therefore, it was considered whether and how this residue would be important to the biosynthesis and activity of this new lasso peptide. The previous RNAP-targeting lasso peptides ubonodin, citrocin, MccJ25, acinetodin, and klebsidin have been 8-membered (8-residue) rings, whereas cloacaenodin has a 9-membered (9-residue) ring (lighter font in FIG. 1B).

### Heterologous Expression of Cloacaenodin

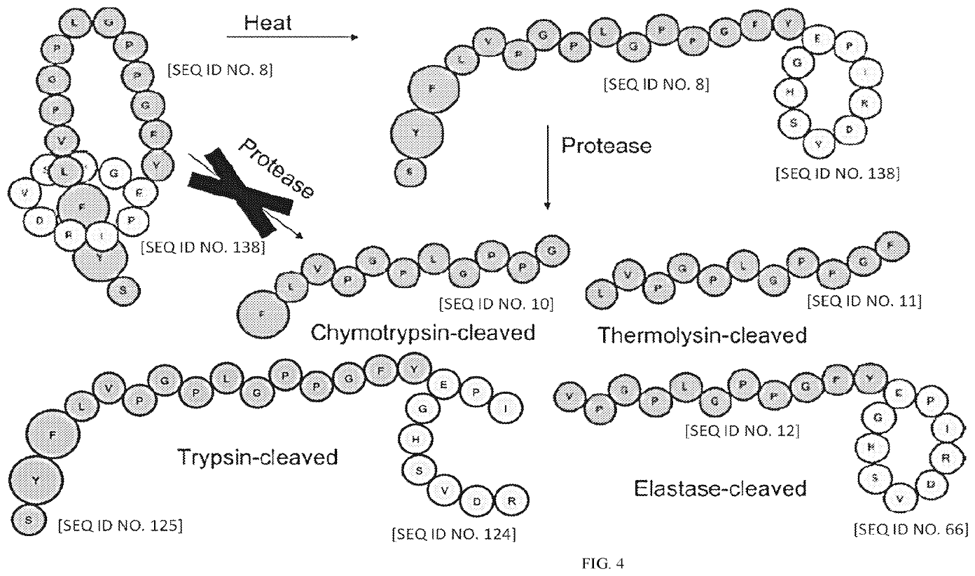

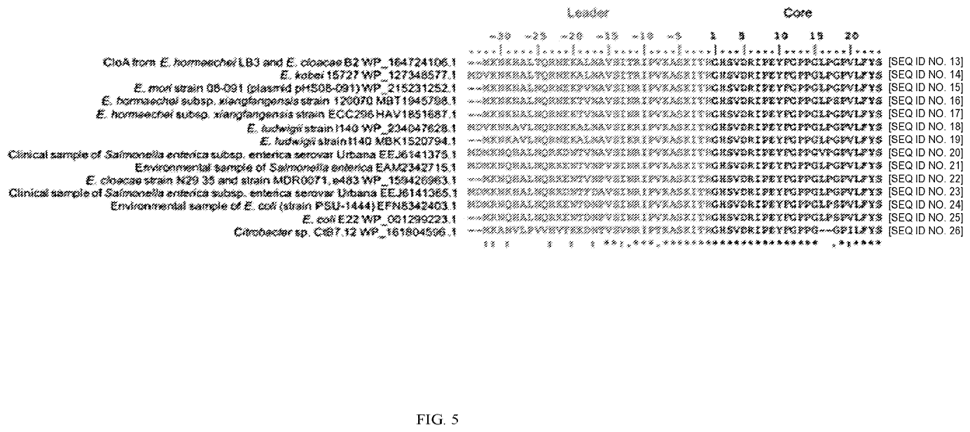

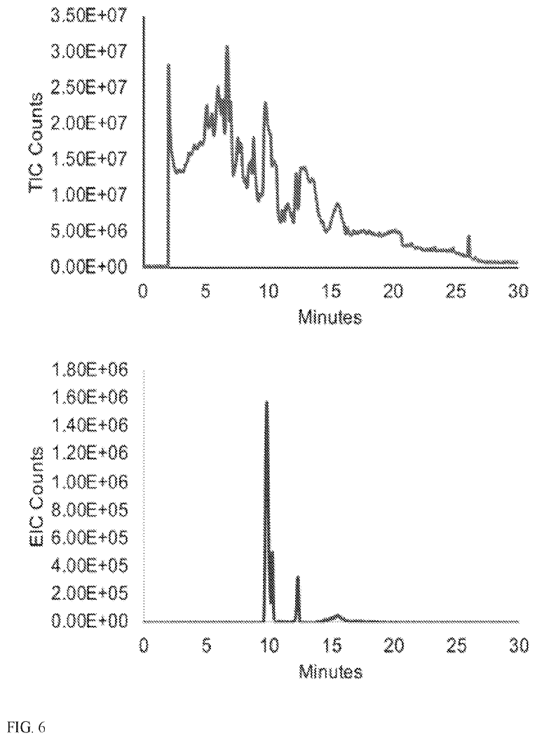

To produce the lasso peptide, a heterologous expression strategy in Escherichia coli, a strategy that has worked for proteobacterial lasso peptide BGCs, was used.^{45,46} The A gene was placed under the inducible T5 promoter in the pQE-80 plasmid with the other genes (B, C, and D) under the control of the constitutive p_{mcjBCD} promoter (FIG. 1A). To facilitate expression and eliminate any codon bias, the gene sequences were codon optimized for Escherichia coli. The resulting codon-optimized BGC was synthesized as gBlocks and cloned into pQE-80. With this construct, cloacaenodin at room temperature in M9 minimal media was expressed overnight. Consistent with the presence of a D gene, which encodes for an adenosine triphosphate (ATP)-binding cassette transporter that exports the lasso peptide from cells, a signal that matched the expected mass of cloacaenodin in the supernatant extract was detected through liquid chromatography-mass spectrometry (LC-MS) analysis. When extracting for the expected cloacaenodin mass a prominent peak and a much smaller later-eluting peak (FIG. 6) were observed. Without being bound by theory, because lasso peptides are capable of unthreading⁴⁷⁻⁴⁹, it was hypothesized that these peaks corresponded to the threaded and unthreaded variants of cloacaenodin.

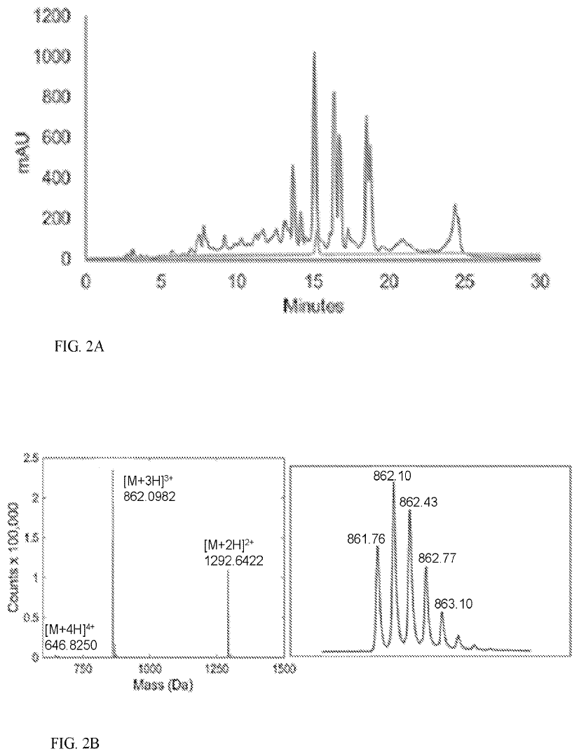

The supernatant extract was injected for high-performance liquid chromatography (HPLC) and the prominent peak eluting at a retention time of 15.0 min (about 60/40 water/acetonitrile) was collected (FIG. 2A). Following collection, this compound was checked by LC-MS, revealing a single peak that eluted at ˜10.5 minutes and contained a species consistent with the expected mass of cloacaenodin (FIG. 2B). A yield of ˜1.1 mg/L of culture was obtained.

More information on the heterologous expression and purification of cloacaenodin is in the sections “Cloning and Plasmid Construction” and “Expression and Purification of Cloacaenodin and Mutants” under “Methods”, below.

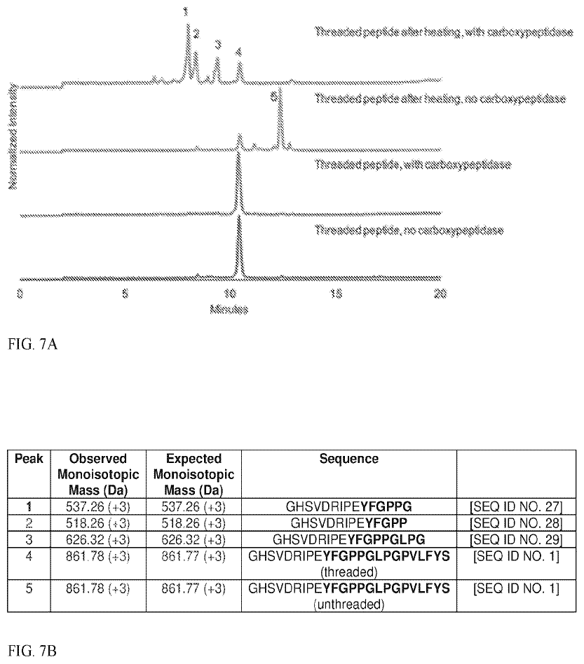

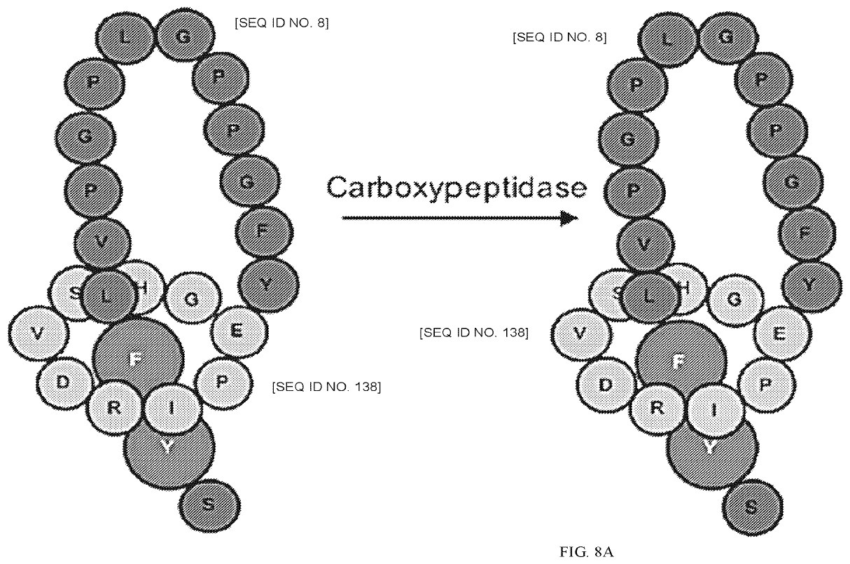

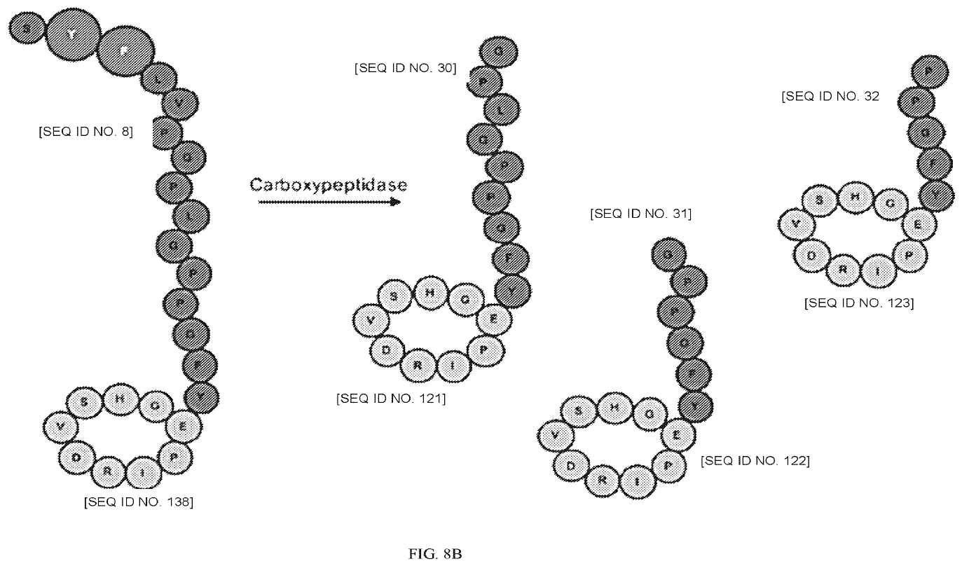

The threaded nature of the collected cloacaenodin sample was next sought to be verified, because the threaded structure is crucial to the bioactivity of lasso peptides.⁵⁰ Heat treatment of certain lasso peptides leads to their unthreading: the unthreaded species often elutes differently than the threaded peptide.^{48,49} We performed a heating assay at 95° C. and noticed that after 1.5 hours ˜5% of cloacaenodin eluted at around 12.4 min instead of 10.5 min (FIGS. 7A-7B). Upon treatment with carboxypeptidase^{48,49}, this later eluting peak was eliminated, and various earlier-eluting peaks consistent with C-terminal truncations of 6, 9, or 10 amino acids were then observed. These truncations were not observed for the unheated cloacaenodin sample when treated with carboxypeptidase (FIGS. 7A-8B). These results support the smaller, and later eluting, peak in the supernatant extract being unthreaded cloacaenodin. This unthreaded variant in the extract may have formed because of thermal unthreading during expression and purification, exposure to organic solvents during purification, or both. To ensure that the sample we collected from the HPLC remained stable, we froze the sample immediately upon collection until lyophilization, such that the peptide was minimally kept in solution.

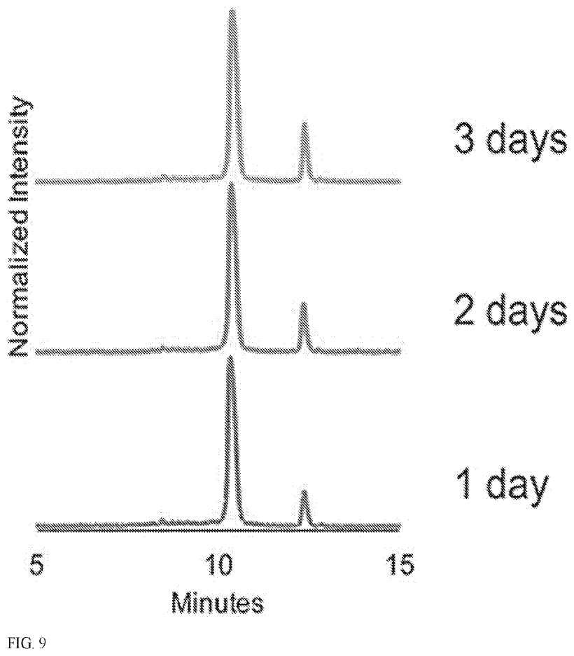

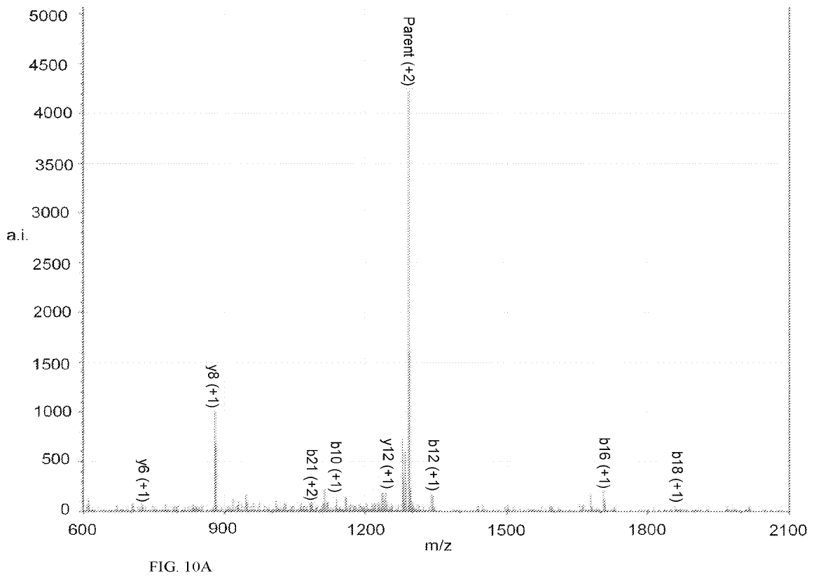

With the finding that cloacaenodin was susceptible to unthreading, it was tested whether and how cloacaenodin would unthread at 37° C., human physiological temperature, in pure water. After a period of 72 hours, the sample of 18 μM cloacaenodin remained ˜83% threaded based on relative peak area on LC-MS (FIG. 9). It may be unusual for lasso peptides to unthread at this temperature: however, in terms of bioactivity, this degree of unthreading stills provide a majority of threaded peptide to exert its function.

More information on testing of cloacaenodin's stability is in the section “Cloacaenodin Stability” under “Methods”, below.

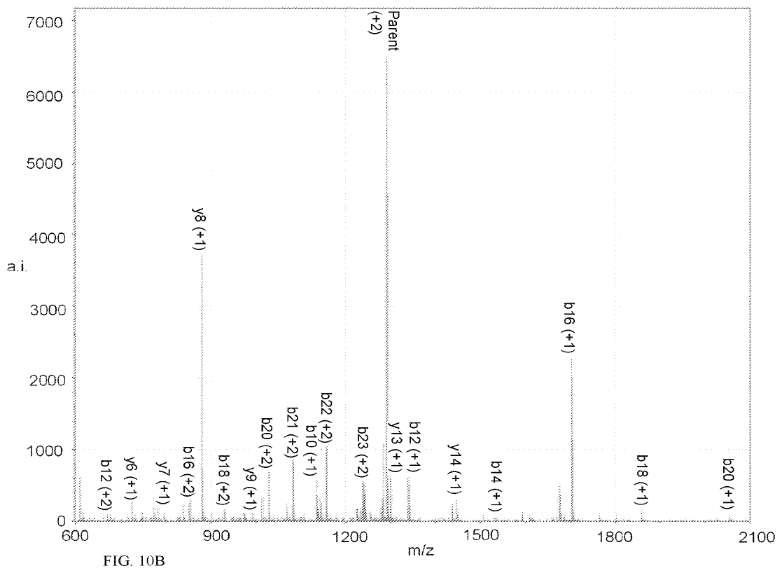

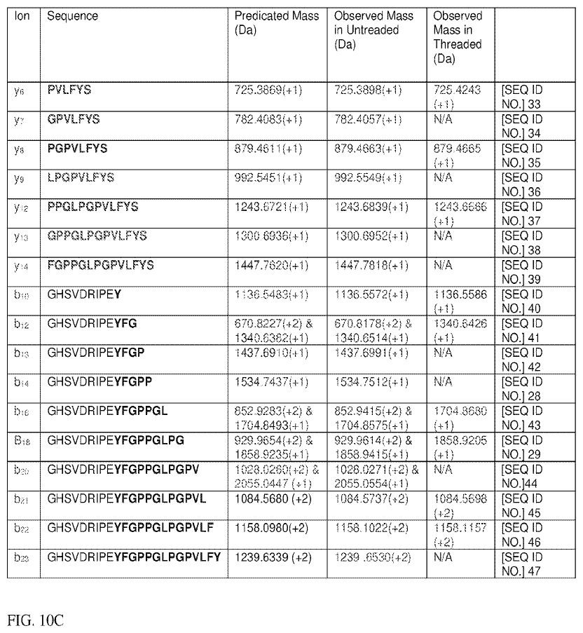

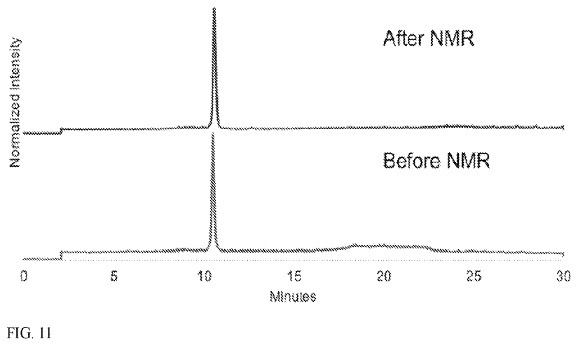

Tandem mass spectrometry (MS/MS) analysis via collision-induced dissociation (CID) was performed on both the threaded and unthreaded conformers of cloacaenodin. Similar fragments were observed for both conformers: however, the unthreaded conformer was more prone to fragmentation than the threaded conformer, consistent with previous observations that threaded lasso peptides are resistant to fragmentation (FIGS. 10A-10B).^{14,16,50} Certain fragmentation ions were only observable for the unthreaded conformer (FIG. 10B).

### NMR Determined Structure of Cloacaenodin

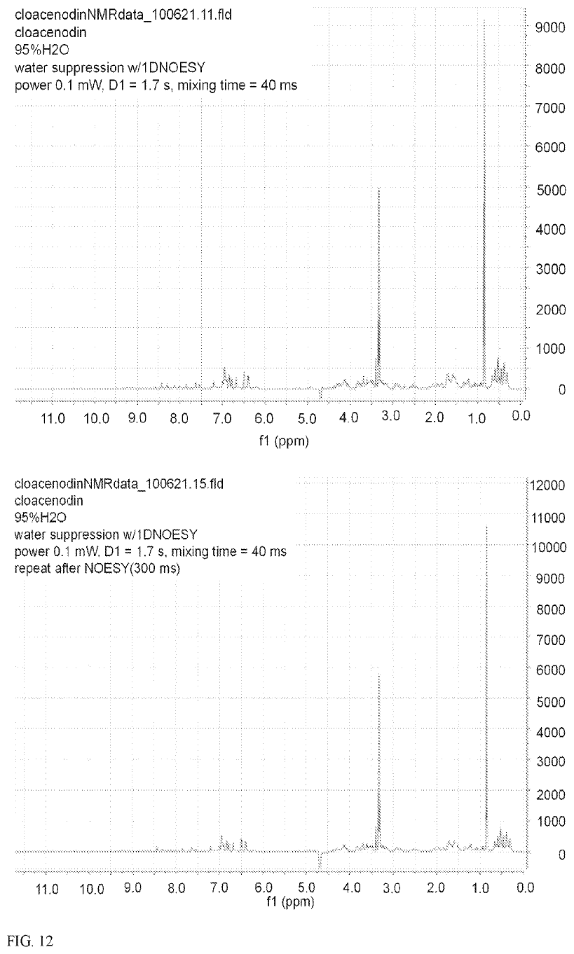

The structure of cloacaenodin in water was determined using two-dimensional (2D) nuclear magnetic resonance (NMR) analysis, a technique that has been successfully used to show the threaded shape of lasso peptides.⁵¹ The possibility of cloacaenodin unthreading during the NMR acquisition was addressed by keeping the sample at 4° C. throughout acquisition. LC-MS analysis of the sample prior to and following NMR data collection confirmed that cloacaenodin remained threaded (FIG. 11). As an additional control, one-dimensional (1D) NMR spectra were acquired in between each 2D NMR spectrum acquisition to ensure stability of the sample (FIG. 12).

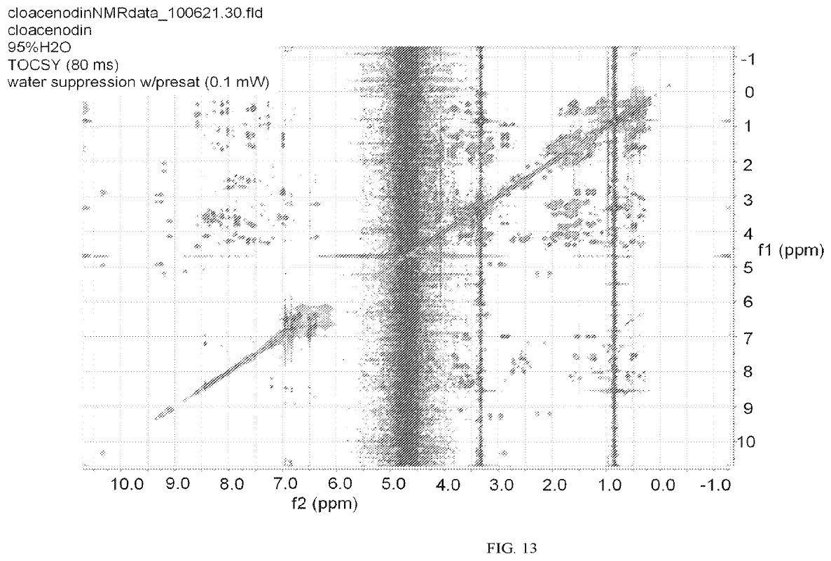

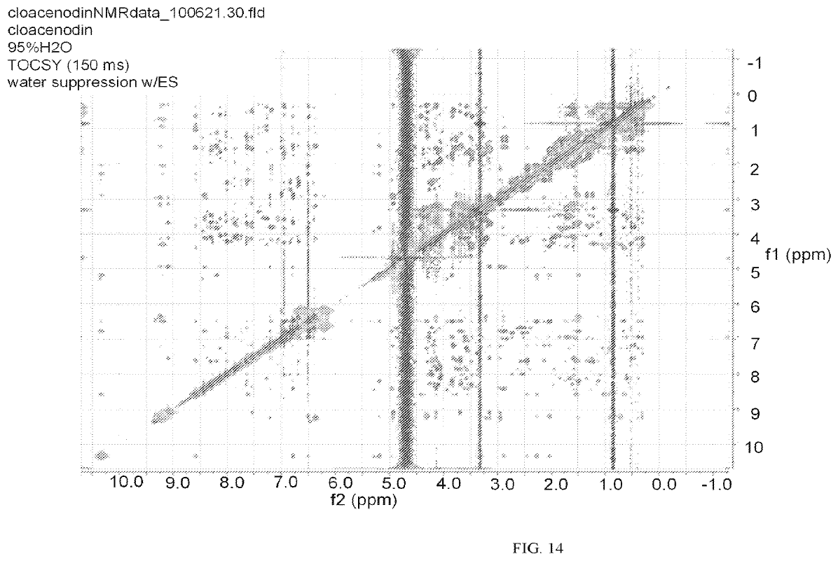

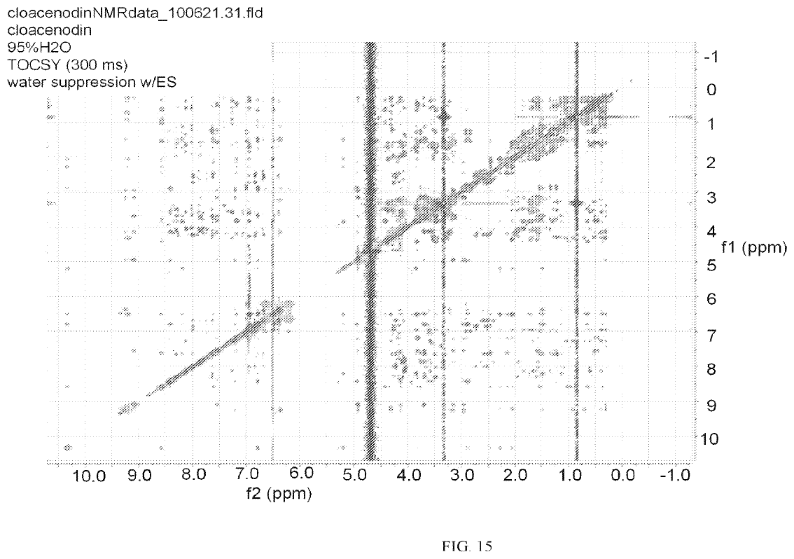

Total correlation spectroscopy (TOCSY) data at a mixing time of 80 ms (FIG. 13) along with two nuclear Overhauser effect spectroscopy (NOESY) spectra were acquired: one at 150 ms (FIG. 14) and one at 300 ms (FIG. 15). The TOCSY and 300 ms NOESY data were used together to assign proton shifts (Table 4). This NMR analysis was particularly challenging because of the presence of five prolines (Pro, P) in the sequence. In addition to the prolines disrupting the NOESY walk during the assignment of residues, these prolines can lead to multiple isomers of cloacaenodin due to cis/trans isomerization as observed previously for MccJ25.⁵² Multiple possibilities in the assignments of Pro17 and Gly 18 (Table 4) were found, and because of this ambiguity in assigning the chemical shifts for these protons, these residues were left undefined in model building. The chemical shifts for the amide protons of Phe22 and Tyr23 (9.210 ppm and 10.320 ppm respectively) are markedly downshifted from the BMRB average: the unique chemical environment experienced by these residues suggest that they may be the amino acids passing through the isopeptide bonded ring. The wide dispersion of the amide proton chemical shifts in the spectra (6.364 ppm-10.320 ppm) provides further support for the lassoed structure of cloacaenodin, because amide proton shifts for unthreaded lasso peptides are generally in a narrower range.⁵¹

TABLE 4
Chemical shift assignments for NMR in water.

| Residue | Hydrogen Atom | δ (ppm) |
|---|---|---|
| Glycine 1 | H | 6.364 |
| | A1 | 4.141 |
| | A2 | 3.222 |
| Histidine 2 | H | 9.243 |
| | A | 4.932 |
| | B2 | 3.148 |
| | B3 | 2.960 |
| | D2 | 7.203 |
| | E1 | 8.421 |
| Serine 3 | H | 9.094 |
| | A | 4.134 |
| | B | 3.697 |
| Valine 4 | H | 7.542 |
| | A | 3.823 |
| | B | 1.576 |
| | QG1 | 0.520 |
| | QG2 | 0.449 |
| Aspartic Acid 5 | H | 7.175 |
| | A | 4.191 |
| | B2 | 2.444 |
| | B3 | 2.139 |
| Arginine 6 | H | 7.612 |
| | A | 3.758 |
| | B2 | 1.342 |
| | B3 | 1.231 |
| | D2 | 2.912 |
| | D3 | 2.834 |
| | QG | 1.529 |
| | E | 6.992 |
| Isoleucine 7 | H | 8.578 |
| | A | 4.417 |
| | B | 1.023 |
| | G12 | 0.966 |
| | G13 | 0.875 |
| | QD | 0.872 |
| | QG | 0.646 |
| Proline 8 | A | 3.835 |
| | B2 | 1.847 |
| | B3 | 1.718 |
| | G2 | |
| | G3 | 1.296 |
| | QD | 3.477 |
| Glutamic Acid 9 | H | 8.189 |
| | A | 3.688 |
| | QB | 1.130 |
| | QG | 1.482 |
| Tyrosine 10 | H | 7.631 |
| | A | 4.217 |
| | B2 | 2.570 |
| | B3 | 2.513 |
| | QD | 6.678 |
| | QE | 6.387 |
| Phenylalanine 11 | H | 7.996 |
| | A | 4.250 |
| | B2 | 2.726 |
| | B3 | 2.516 |
| | QD | 6.504 |
| | QE | 6.945 |
| | HZ | |
| Glycine 12 | H | 6.945 |
| | QA | 3.606 |
| Proline 13 | A | 4.373 |
| | B2 | 2.050 |
| | B3 | 1.717 |
| | G2 | |
| | G3 | 1.599 |
| | QD | 3.265 |
| Proline 14 | A | 4.171 |
| | B2 | 2.001 |
| | B3 | 1.717 |
| | G2 | |
| | G3 | 1.552 |
| | QD | 3.312 |
| Glycine 15 | H | 8.267 |
| | A2 | 3.664 |
| | A3 | 3.477 |
| Leucine 16 | H | 8.018 |
| | A | 4.293 |
| | B2 | 1.215 |
| | B3 | 1.145 |
| | QQD | 0.504 |
| Proline 17 | A | 4.115 |
| | B2 | 1.937/1.834 |
| | B3 | 1.695/1.595 |
| | QG | 1.553/1.346 |
| | QD | 3.323/3.324 |
| Glycine 18 | H | 7.856/8.303 |
| | A2 | 3.758/3.688 |
| | A3 | 3.688/3.487 |
| Proline 19 | A | 4.067 |
| | B2 | 1.834 |
| | B3 | 1.595 |
| | G2 | |
| | G3 | 1.346 |
| | QD | 3.172 |
| Valine 20 | H | 8.123 |
| | A | 3.579 |
| | B | 1.660 |
| | QG1 | 0.594 |
| | QG2 | 0.381 |
| Leucine 21 | H | 7.264 |
| | A | 4.315 |
| | B2 | 1.052 |
| | B3 | 0.919 |
| | G | 0.708 |
| | QD1 | 0.379 |
| | QD2 | 0.309 |
| Phenylalanine 22 | H | 9.210 |
| | A | 5.190 |
| | B2 | 2.271 |
| | B3 | 2.045 |
| | QC | |
| | QE | |
| | HZ | |
| Tyrosine 23 | H | 10.320 |
| | A | 4.955 |
| | QB | 2.890 |
| | QD | 6.780 |
| | QE | 6.485 |
| Serine 24 | H | 8.420 |
| | A | 4.134 |
| | B2 | 3.613 |
| | B3 | 3.547 |

Both shift assignments are listed for Pro17 and Gly18; however, because of ambiguity these were left out in model building. Other shifts are listed for protons that could be unambiguously assigned for input into the CYANA analysis program.

The 150 ms NOESY spectrum was integrated for through-space distance restraints. From the NOESY spectrum, interactions between protons from Phe22 (F22) with Asp5 (D5), Arg6 (R6), Pro8 (P8), and Glu9 (E9), as well as between protons from Tyr23 (Y23) with Gly1 (G1), His2 (H2), Asp5 (D5), Ile7 (17), Pro8 (P8), and Glu9 (E9), were observed (Table 5). These interactions indicated that Phe22 and Tyr23 likely function as the bulky steric lock residues to keep the peptide threaded. These assignments, as well as through-space NOEs from integrated peaks and explicit distance constraints around the isopeptide bond (calculated from the rubrivinodin crystal structure PDB 50QZ)⁵³, were given to the CYANA 2.1 analysis program⁵⁴ using the automated mode, where all prolines (Pro, P) were presumed trans.

TABLE 5
NOEs observed between lock and ring residues.

| Position | Residue | Hydrogen | Position | Residue | Hydrogen |
|---|---|---|---|---|---|
| 23 | Tyr | H | 5 | Asp | HB2 |
| 23 | Tyr | H | 5 | Asp | HB3 |
| 23 | Tyr | H | 7 | Ile | HG12 |
| 23 | Tyr | H | 9 | Glu | QG |
| 23 | Tyr | H | 8 | Pro | HA |
| 23 | Tyr | QD | 9 | Glu | H |
| 23 | Tyr | HA | 2 | His | HD2 |
| 23 | Tyr | QD | 2 | His | HD2 |
| 23 | Tyr | QB | 2 | His | HD2 |
| 23 | Tyr | QC | 9 | Glu | QG |
| 23 | Tyr | QD | 9 | Glu | QB |
| 23 | Tyr | QD | 7 | Ile | HG12 |
| 23 | Tyr | QD | 7 | Ile | QG2 |
| 23 | Tyr | QD | 9 | Glu | HA |
| 23 | Tyr | QE | 7 | Ile | QG2 |
| 23 | Tyr | QE | 9 | Glu | HA |
| 23 | Tyr | QE | 9 | Glu | QG |
| 23 | Tyr | QB | 1 | Gly | H |
| 23 | Tyr | HA | 5 | Asp | HB2 |
| 23 | Tyr | HA | 5 | Asp | HB3 |
| 23 | Tyr | QB | 1 | Gly | HA2 |
| 22 | Phe | HB2 | 7 | Ile | H |
| 22 | Phe | HB3 | 7 | Ile | H |
| 22 | Phe | HA | 9 | Glu | H |
| 22 | Phe | HB2 | 6 | Arg | H |
| 22 | Phe | HB2 | 5 | Asp | H |
| 22 | Phe | HA | 8 | Pro | HA |
| 22 | Phe | HA | 9 | Glu | QB |
| 22 | Phe | HA | 9 | Glu | QG |
| 22 | Phe | HB2 | 8 | Pro | HA |
| 22 | Phe | HB3 | 8 | Pro | HA |
| 22 | Phe | HB2 | 5 | Asp | HB3 |
| 22 | Phe | HB3 | 5 | Asp | HB3 |
| 22 | Phe | HB2 | 5 | Asp | HB2 |

More information on the acquisition of NMR data is in the sections “NMR Data Collection” and “Determination of Structure through NMR Analysis” under “Methods”, below.

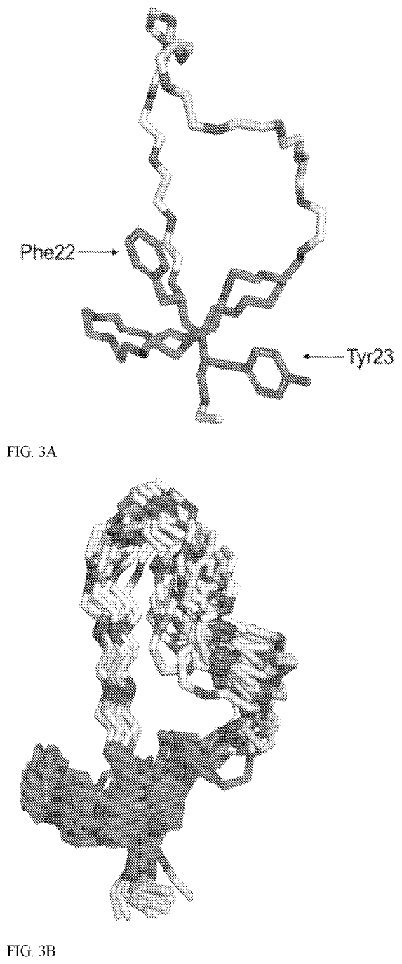

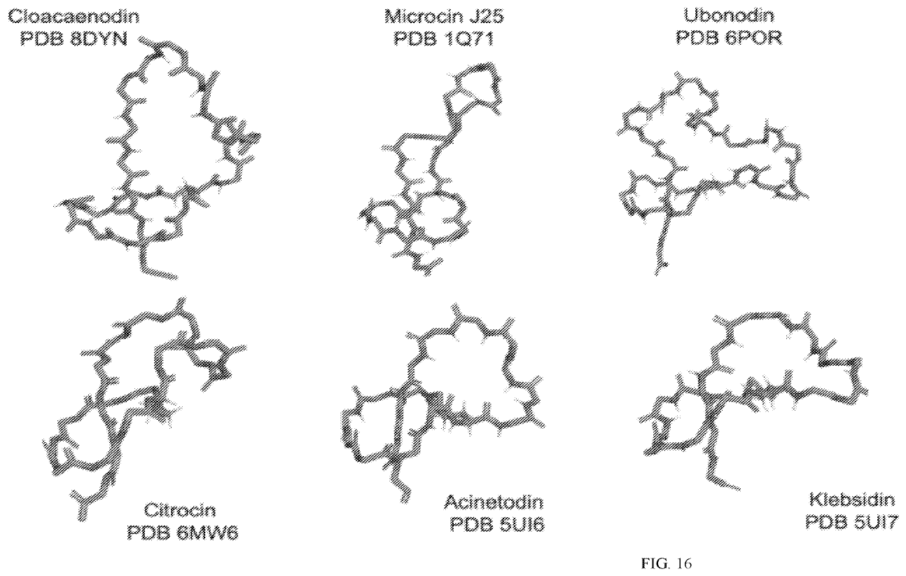

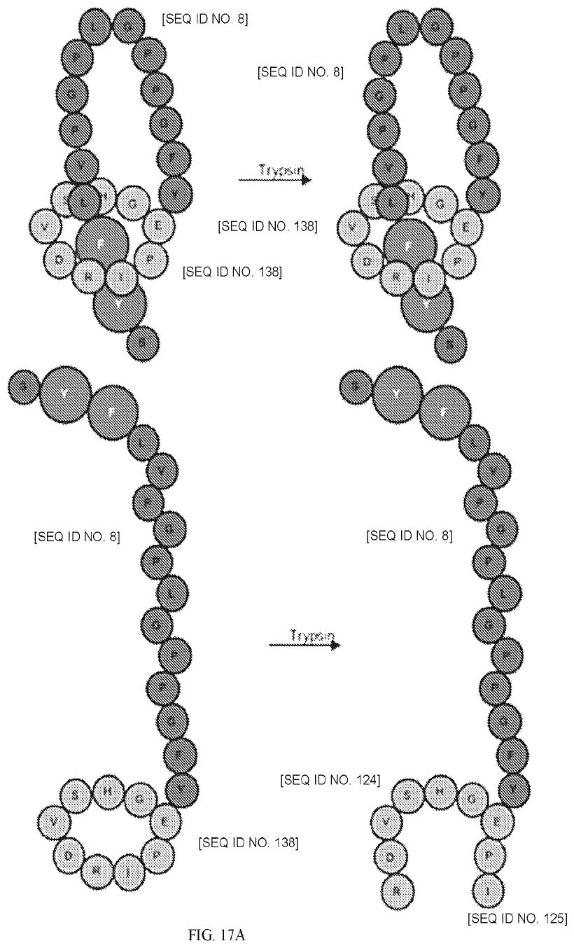

The top 20 structures calculated by CYANA show Phe22 and Tyr23 as the upper and lower steric lock residues respectively (FIG. 3A), similar to the Phe19 and Tyr20 steric locks of MccJ2550,55,56. Cloacaenodin has a loop region of 13 amino acids (YFGPPGLPGPVLF [SEQ ID NO. 115]), which is the second largest loop observed after ubonodin, and a short tail region of only two amino acids (YS) (FIG. 3B). This large loop and short tail structure is similar to that of the RNAP-inhibiting lasso peptides MccJ25, ubonodin, citrocin, klebsidin, and acinetodin. For example, cloacaenodin is unique with its C-terminal serine (Ser, S) and 9-membered (9-residue) ring (FIG. 16).

### Threaded Cloacaenodin Resists Protease Cleavage

An advantage of a lasso peptide is that it may be resistant to proteolysis. Cloacaenodin was determined to be resistant to C-terminal proteolysis by the exopeptidase carboxypeptidase (FIGS. 7A-8B). It was investigated whether the lasso peptide cloacaenodin was resistant to endopeptidases.

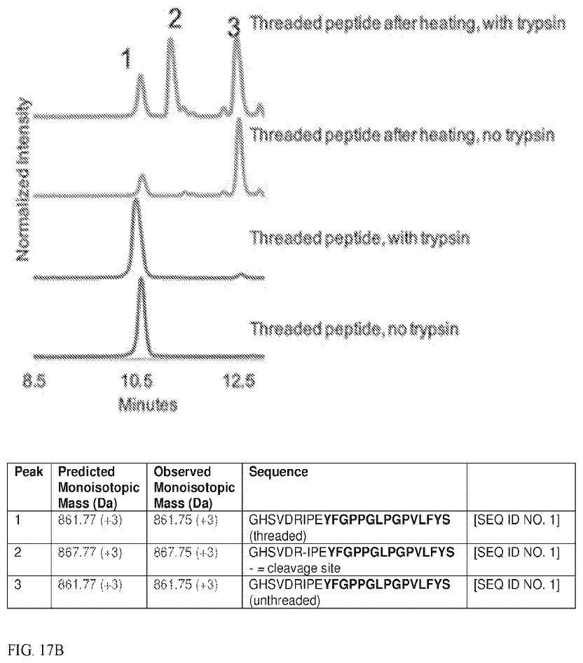

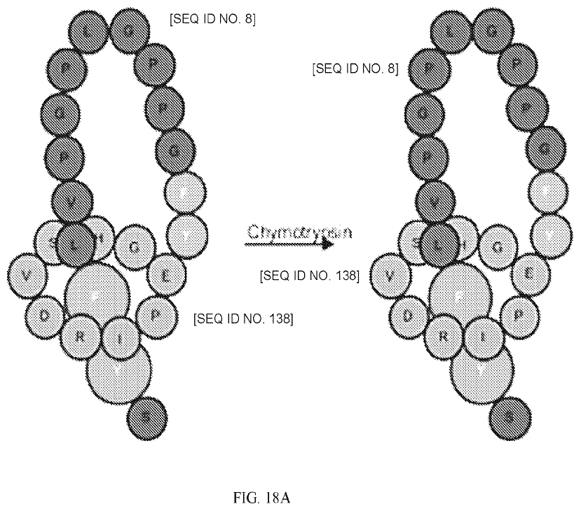

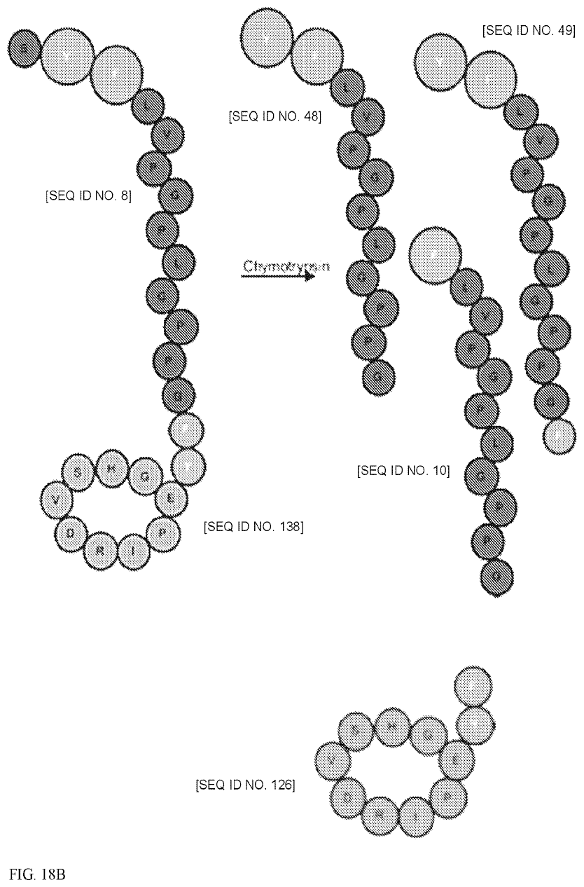

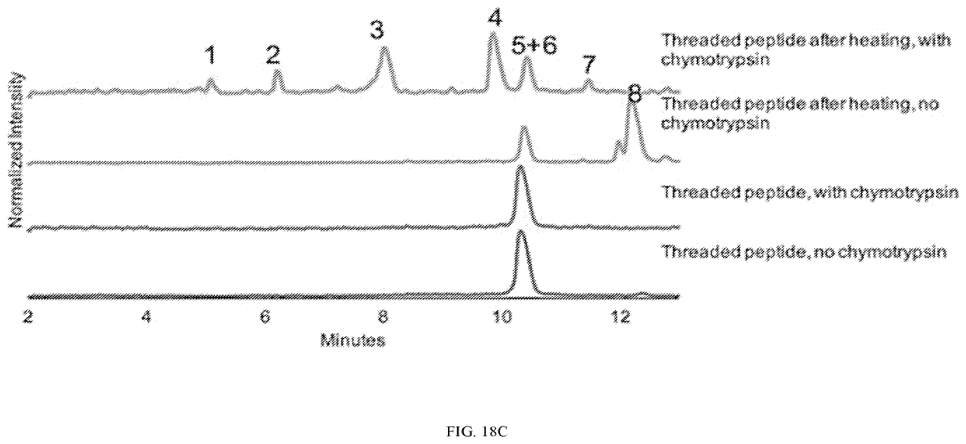

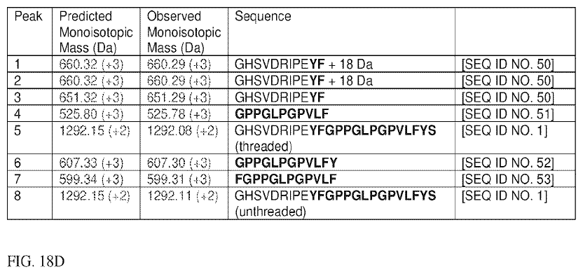

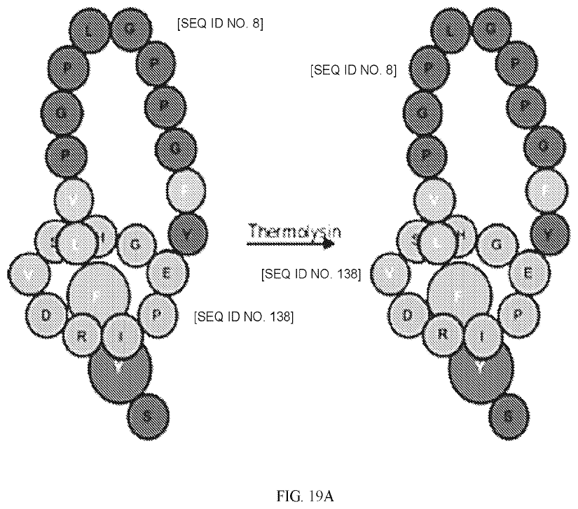

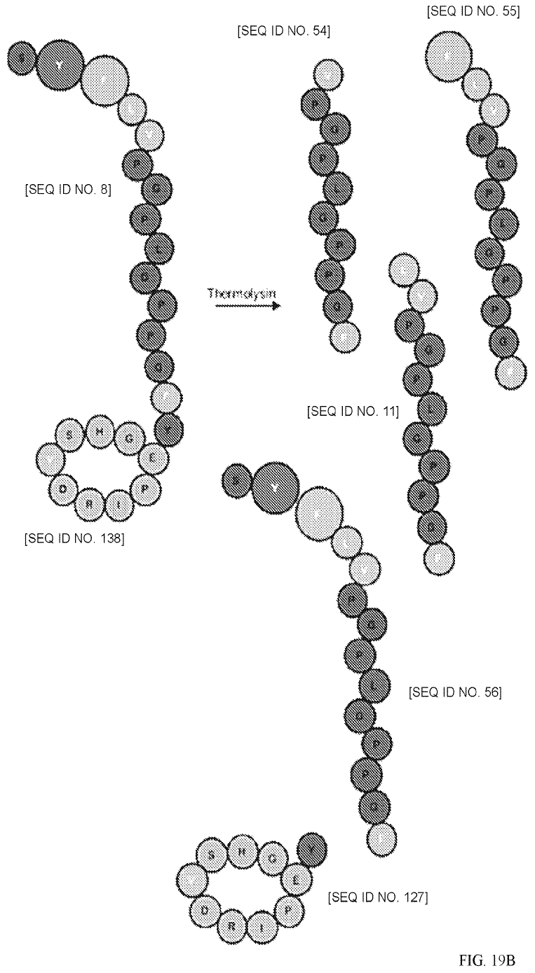

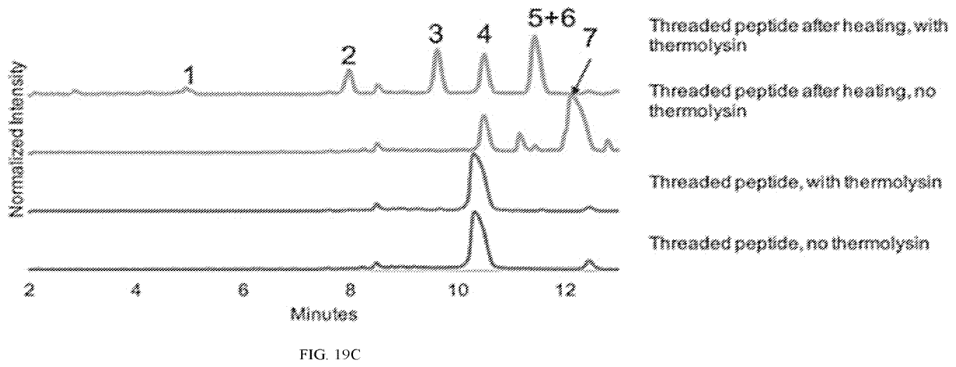

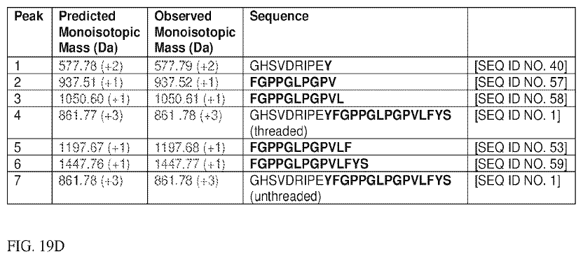

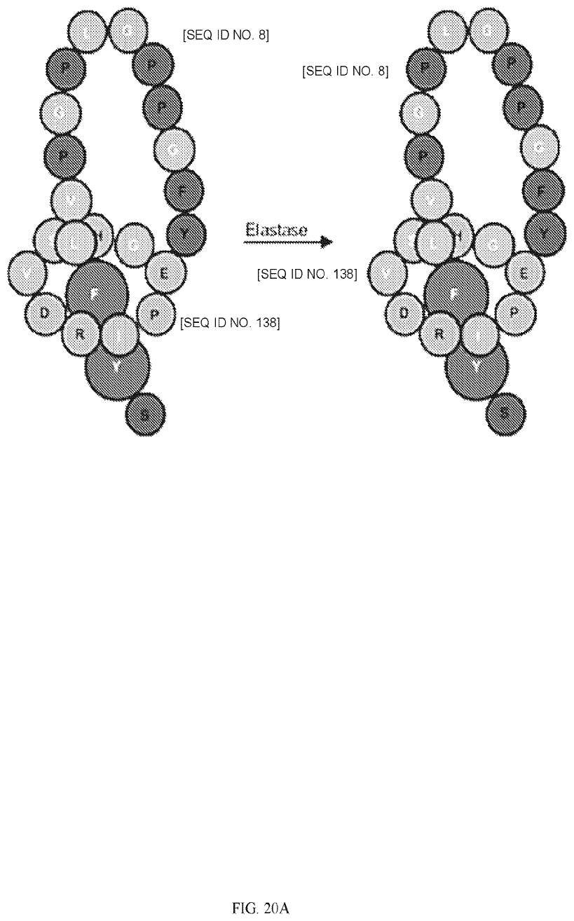

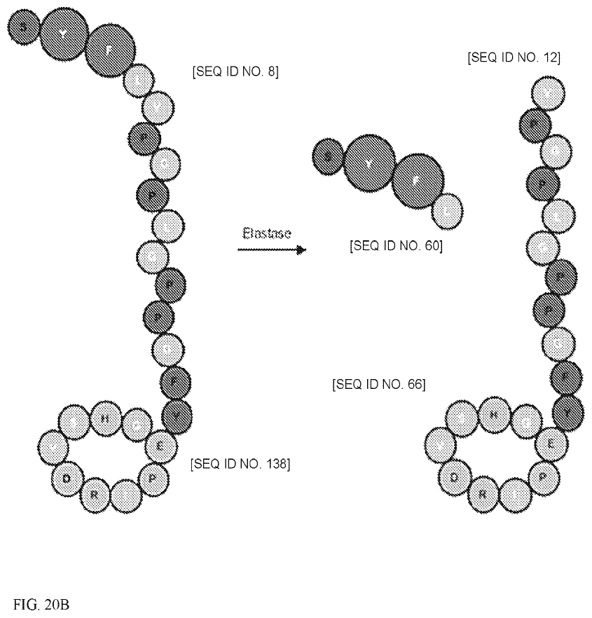

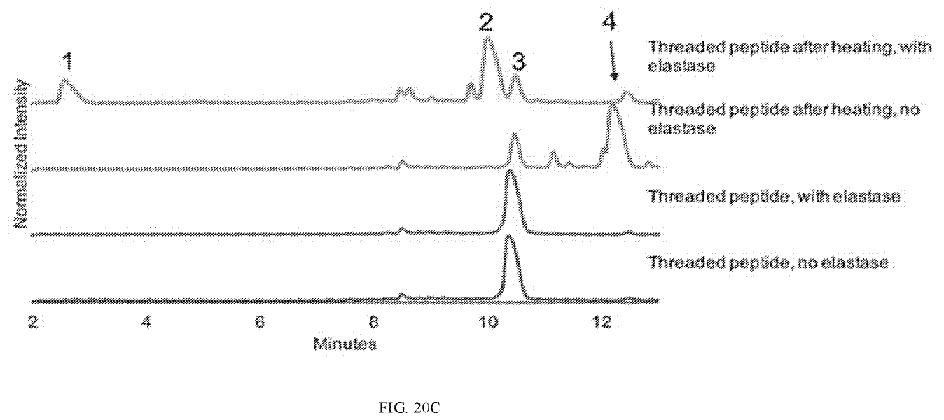

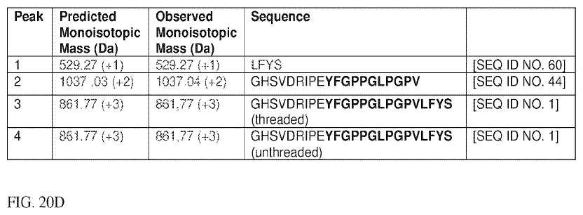

The sequence of cloacaenodin contains residues (amino acid residues) cleavable by trypsin, chymotrypsin, elastase, and thermolysin. When testing was done on a sample of unthreaded cloacaenodin (generated by heating), fragments consistent with cleavage of the unthreaded peptide both in its linear and ring segments were observed. In contrast, under the same conditions, the threaded cloacaenodin lasso peptide remained resistant to proteolysis by each of these four proteases (FIGS. 4 and 17A-20D). This is consistent with the proteolytic resistance of other lasso peptides and demonstrates that the threaded structure of cloacaenodin is essential to its proteolytic stability.⁵⁷⁻⁵⁹

More information on the testing of resistance of cloacaenodin to proteolysis is in the sections “Carboxypeptidase Digestion” and “Protease Digestion” under “Methods”, below.

### Cloacaenodin has Antimicrobial Activity Against Multiple Enterobacter Strains

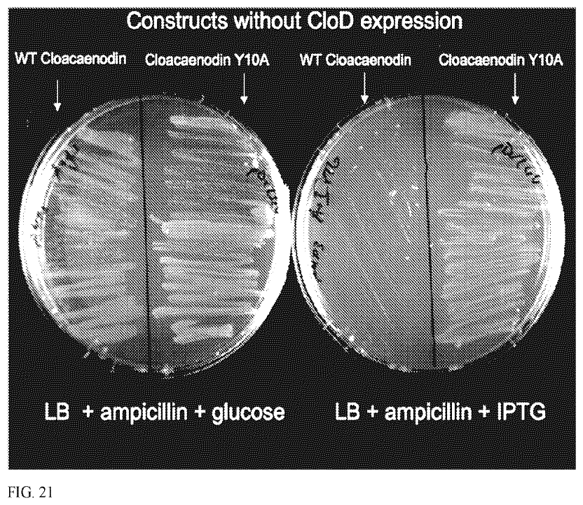

After characterizing the structure and proteolytic resistance of cloacaenodin, cloacaenodin's inhibition of bacterial growth was tested. As a preliminary test of antimicrobial activity, a version of the expression plasmid from which the cloD gene was removed was cloned, so that the lasso peptide could not be exported by the Escherichia coli upon isopropyl β-D-1-thiogalactopyranoside (IPTG)-induced expression. Escherichia coli XL-1 Blue colonies did not appear on B agar when IPTG was added to the plate (FIG. 21), indicative of intracellular cloacaenodin toxicity.

Among antimicrobial lasso peptides, target strains may be phylogenetically or environmentally related to the producing strain.⁶⁰ Strains that were members of the Enterobacter cloacae complex (ECC), as well as strains that may reside in the same environmental niche as Enterobacter strains, were tested. There are different proposed names for some of the strains tested, such as the re-classification of Enterobacter amnigenus and Enterobacter nimipressuralis to the genus Lelliottia.⁶¹

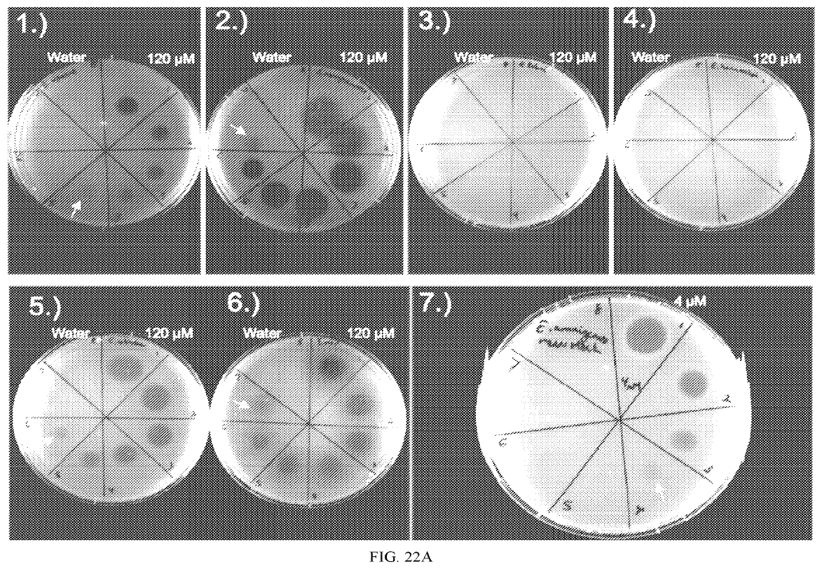

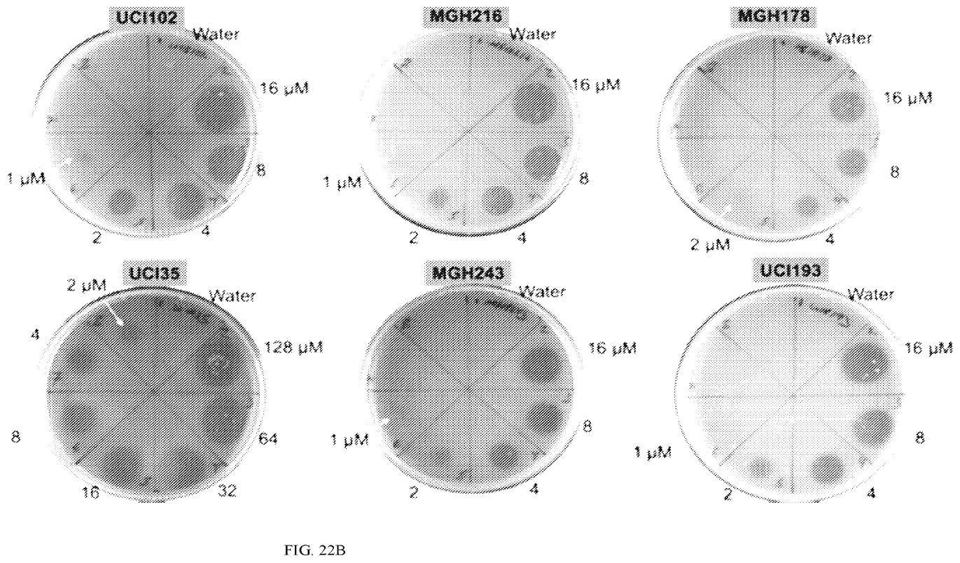

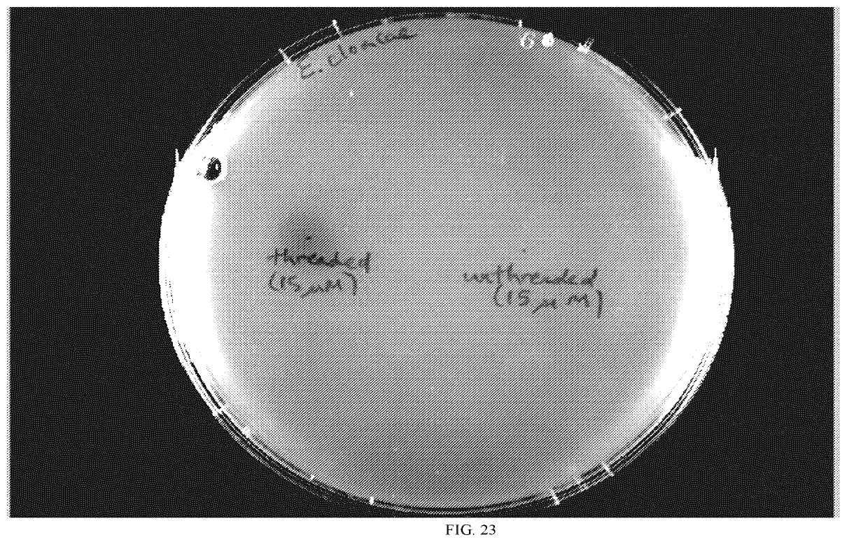

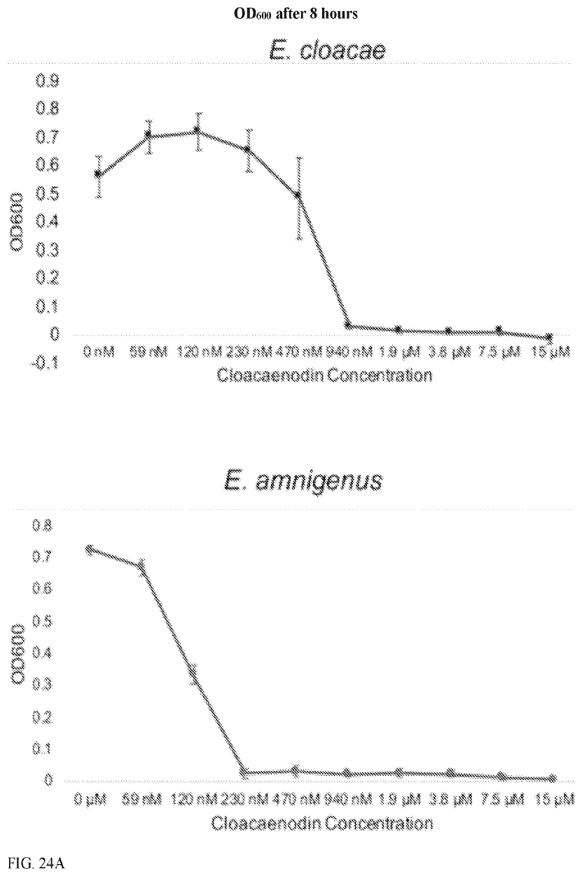

Spot-on-lawn assays in M63 agar against a panel of bacteria using purified cloacaenodin dissolved in water (FIG. 22A, Table 6) were used. Cloacaenodin had no observed activity against E. coli MG1655, B. subtilis 168, Salmonella enterica serovar Newport, Klebsiella aerogenes ATCC 13048, Enterobacter hormaechei ATCC 700323, or Enterobacter kobei BAA-260. However, zones of inhibition in the low micromolar range of cloacaenodin concentration were observed for cloacaenodin tested against Enterobacter cloacae ATCC 13047, Enterobacter mori DSM 26271, Enterobacter asburide DSM 17506, Enterobacter amnigenus ATCC 33072, and Enterobacter nimipressuralis DSM 18955 (Table 8). Enterobacter cloacae may be the most clinically relevant strain against which activity was found, as this pathogen is a frequent cause of nosocomial infections around the globe.² The other strains found to be susceptible to cloacaenodin are causative agents for certain plant diseases or human pathogens.⁶²⁻⁶⁵

TABLE 6
Commercial strains or laboratory strains tested for cloacaenodin activity.
Strain

* Enterobacter cloacae subsp. cloacae
ATCC 13047
Klebsiella aerogenes
ATCC 13048
Enterobacter hormaechei
ATCC 700323
* Enterobacter asburiae
DSM 17506
* Enterobacter mori
DSM 26271
Enterobacter kobei
BAA-260
* Lelliottia nimipressuralis
DSM 18955
* Enterobacter amnigenus
ATCC 33072
Escherichia coli
MG1655
Bacillus subtilis
168
Salmonella enterica sp. enterica
serovar Newport
An asterisk indicates that the strain was susceptible to cloacaenodin.

The type strain of Enterobacter cloacae (ATCC 13047) tested as discussed above was isolated from human cerebrospinal fluid in 1890⁶⁶ before the era of antibiotics. The activity of cloacaenodin was also tested against more recent clinical isolates. A panel of two (2) clinical isolates of Enterobacter using the spot-on-lawn assay was tested (Table 7). These isolates are part of a larger collection of carbapenem-resistant Enterobacterales (CRE) that have been analyzed for their genetic mechanisms of resistance.⁶⁷ In six (6) of these strains, three (3) of which are classified as resistant to carbapenem antibiotics, low micromolar values of the minimal inhibitory concentration (MIC) of cloacaenodin were observed (Tables 7-8, FIG. 22B). In the other clinical isolates, we observed either weak or no activity (Table 7). The observation of strain-specific activity is consistent with the observed narrow-spectrum activity of ubonodin against Burkholderia strains due to transport through PupB.^{16,68} These assays demonstrate that cloacaenodin has the potential to serve as a potent therapeutic against Enterobacter strains that have evolved resistance to last-resort antibiotics.

TABLE 7
Clinical strains tested for cloacaenodin activity.

| | | | | Classified |
|---|---|---|---|---|
| | | | Meropenem | as |
| | | Isolation | MIC | Carbapenem |
| Strain ID | Species | Source | (μg/mL) | Resistant? |
| AR0154 | Enterobacter | N/A | N/A | Yes |
| | cloacae | | | |
| BIDMC120 | Enterobacter | Foot ulcer | 4 | Yes |
| | cloacae | | | |
| BIDMC87 | Enterobacter | Blood | 16 | Yes |
| | hormaechei | culture | | |
| BWH39 | Enterobacter | Urine | 2 | Yes |
| | hormaechei | | | |
| MGH3 | Enterobacter | Respiratory | ≥16 | Yes |
| | cloacae | | | |
| * UCI35 | Enterobacter | Surgical | 4 | Yes |
| | hormaechei | Procedure | | |
| | | Specimen | | |
| * MGH178 | Enterobacter | Wound | ≤0.25 | No |
| | kobei | | | |
| * MGH216 | Enterobacter | Urine | ≤0.25 | No |
| | ludwigii | | | |
| MGH239 | Enterobacter | Urine | 1 | Intermediate |
| | roggenkampii | | | |
| * MGH243 | Enterobacter | Tissue | 16 | Yes |
| | asburiae | Wound | | |
| * UCI102 | Enterobacter | Urine | 0.063 | No |
| | cloacae | | | |
| * UCI193 | Enterobacter | Sputum | 23 | Yes |
| | asburiae | | | |

Strains designated with BIDMC were isolated at Beth Israel Deaconess Medical Center in Boston, MA. Strains designated with BWH were isolated at Brigham and Women's Hospital in Boston, MA. Strains designated with UCI were isolated at University of California in Irvine, CA. Strains designated with MGH were isolated at Massachusetts General Hospital in Boston, MA. AR0154 was obtained from the Antibiotic Resistance Bank from the U.S. Centers for Disease Control. An asterisk indicates that the strain was susceptible to cloacaenodin.

TABLE 8
Minimal inhibitory concentration (MIC) of
cloacaenodin against susceptible strains.

| Species | MIC in M63 media | |
|---|---|---|
| Enterobacter cloacae ATCC 13047 | 7.5 μM | (agar)/ |
| | | 940 nM (liquid) |
| Enterobacter nimipressuralis DSM 18955 | 940 nM | (liquid) |
| Enterobacter asburiae DSM 17506 | 1.9 μM | (agar) |
| Enterobacter mori DSM 26271 | 3.8 μM | (agar) |
| Enterobacter amnigenus ATCC 33072 | 1.9 μM | (agar) |
| * Enterobacter cloacae UCI102 | 500 nM | (agar)/230 nM |
| | | (liquid) |
| * Enterobacter ludwigii MGH216 | 230 nM | (liquid) |
| * Enterobacter ludwigii MGH216 | 230 nM | (liquid) |
| * Enterobacter kobei MGH178 | 1 μM | (agar) |
| * Enterobacter hormaechei UCI35 | 1 μM | (agar) |
| * Enterobacter asburiae MGH243 | 2 μM | (agar) |
| * Enterobacter asburiae UCI193 | 2 μM | (agar) |

An asterisk indicates that the strain is a clinical isolate. Also tested was the bioactivity of a sample of cloacaenodin that had been heated and then HPLC-purified to isolate the unthreaded peptide. At a concentration of 15 μM, no activity of this unthreaded peptide against Enterobacter cloacae was observed, consistent with the threaded structure of the cloacaenodin lasso peptide being essential for activity (FIG. 23).

The bioactivity of a sample of cloacaenodin that had been heated and then HPLC-purified to isolate the unthreaded peptide was tested. At a concentration of 15 μM, no activity of this unthreaded peptide against Enterobacter cloacae was observed, consistent with the threaded structure of the cloacaenodin lasso peptide being essential for activity (FIG. 23).

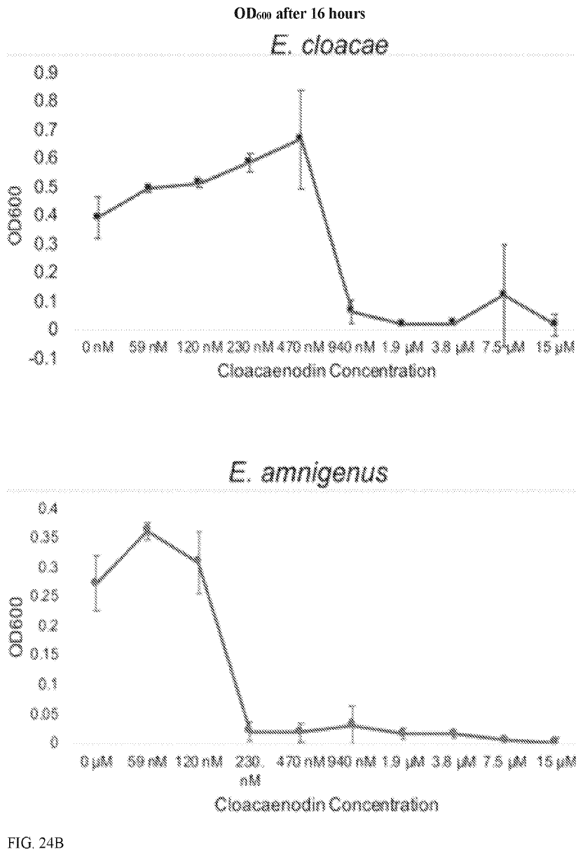

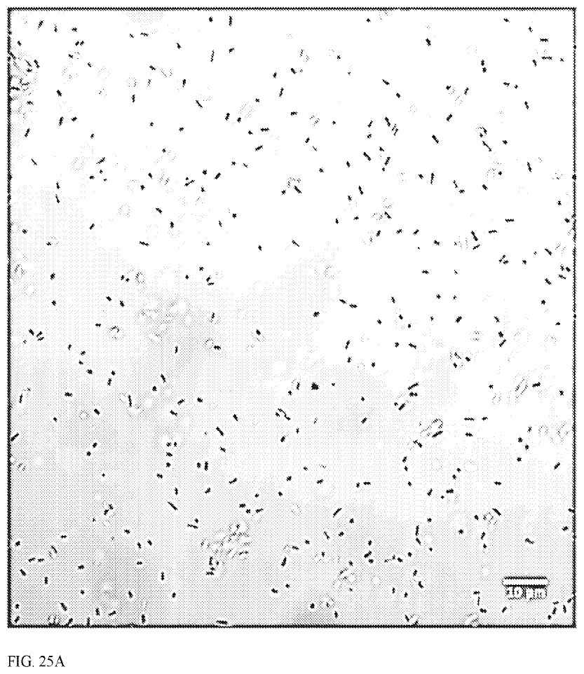

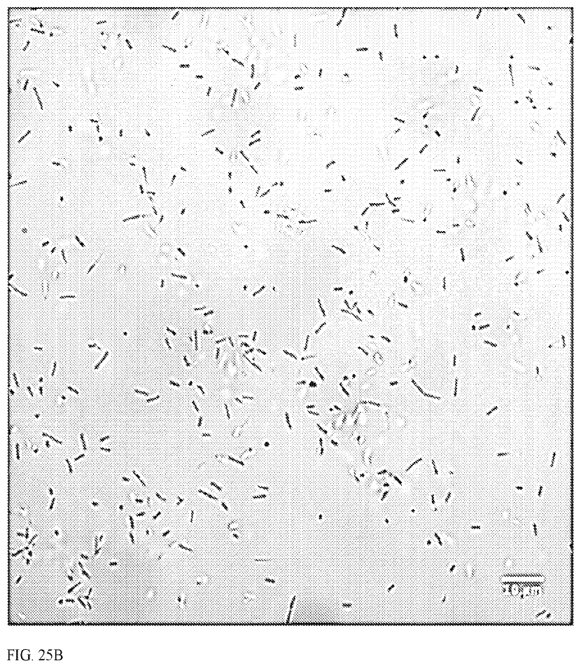

More information on the testing for activity of cloacaenodin against bacterial strains is in the section “Cloacaenodin Antimicrobial Activity” under “Methods”, below. Cloacaenodin was tested against Enterobacter cloacae and Enterobacter amnigenus using a broth microdilution assay in M63 media. An MIC value of 940 nM for Enterobacter cloacae and 230 nM for Enterobacter amnigenus was observed from this assay, showing that cloacaenodin is more active in solution than on solid media (Table 8, FIGS. 24A-24B). When testing against Enterobacter cloacae, greater optical density (OD) values for sub-MIC treated wells were noticed, which motivated looking for a phenotypic abnormality for these cells. Under the microscope, Enterobacter cloacae cells that were treated with sub-MIC concentrations of cloacaenodin were observed to exhibit a degree of filamentation (FIGS. 25A-25B). Filamentation has been observed for Escherichia coli cells upon exposure to MccJ25.¹³ More information on the microscope technique is in the section “Microscopy” under “Methods”, below.

### Stability and Bioactivity of Cloacaenodin Variants

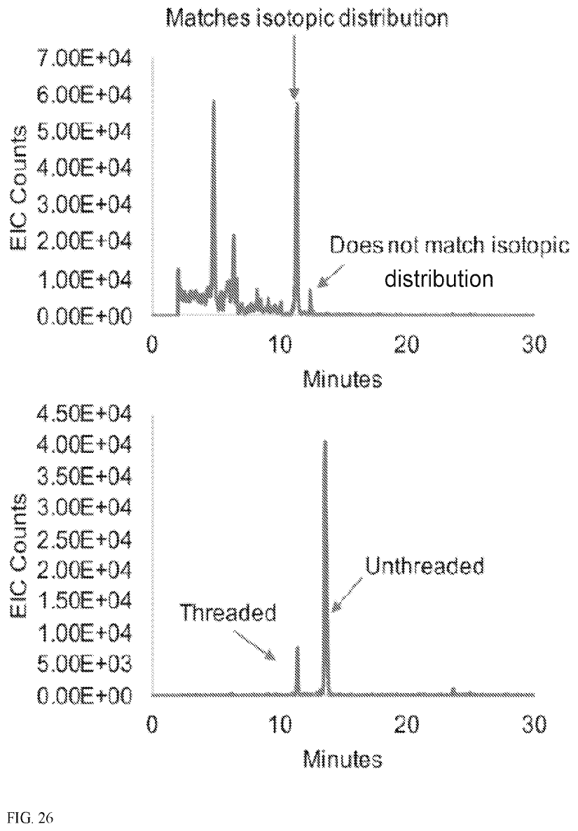

To gain insight into the contribution of specific cloacaenodin residues toward cloacaenodin's stability and bioactivity, site-directed mutagenesis on various residues was carried out, with the main results summarized in Table 9. A peak of unthreaded cloacaenodin was seen in the supernatant extract (FIG. 6). Without being bound by theory, increasing the sidechain volume of the steric lock residues Phe22 (F22) and Tyr23 (Y23) may retard the unthreading process. Swapping of the upper steric lock Phe22 (F22) to Trp22 (W22) still resulted in comparable levels of unthreaded peptide in the extract, while a Y23W (swap of Tyr23 to Trp23) variant only had the threaded peptide observable in its supernatant extract (although the yield was reduced >10-fold) (FIG. 26).

TABLE 9
Summary of yield, stability, and bioactivity
of cloacaenodin variants.

| | Yield | Stability | Bioactivity |
|---|---|---|---|
| Cloacaenodin | Relative | Relative | Relative |
| Variant | to WT | to WT | to WT |
| F22W | ++ | Comparable | Comparable |
| Y23W | + | More | n.d. |
| S24G | +++ | Less | Abolished |
| S24T | ++ | Comparable | Less |
| S24C | ++ | Comparable | Less |
| S24A | ++ | Comparable | Comparable |
| S24Y | ++ | Comparable | Comparable |
| 10-membered | L | Less | n.d. |
| ring | | | |
| G1A | Not tolerated | N/A | N/A |
| E9D | Not tolerated | N/A | N/A |
| P8A | ++ | Comparable | n.d. |
| V4P | L | More | n.d. |
| Y10A | + | Comparable | Much less |

For yield, +++ signifies made at near wild-type levels, ++ signifies made at ~40-60% wild-type levels, + signifies made at ≤10% wild-type levels, all as detected by absorbance at UV-215 measured from HPLC. L signifies only detected on LC-MS. Stability was judged by ratio of threaded-to-unthreaded peptide in supernatant extract after purification, judged by LC-MS. N/A stands for not applicable. Variants that were not tested for bioactivity are listed as n.d. for no data.

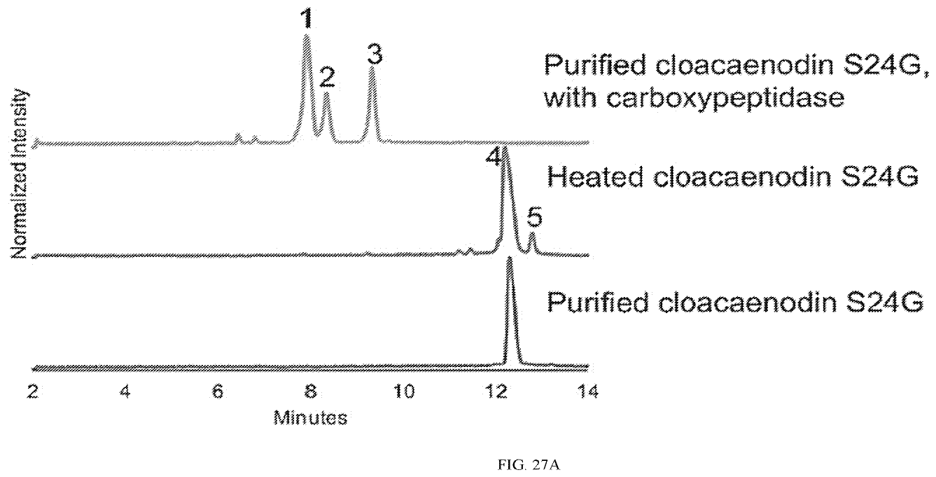

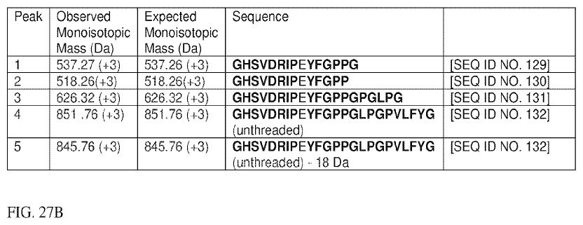

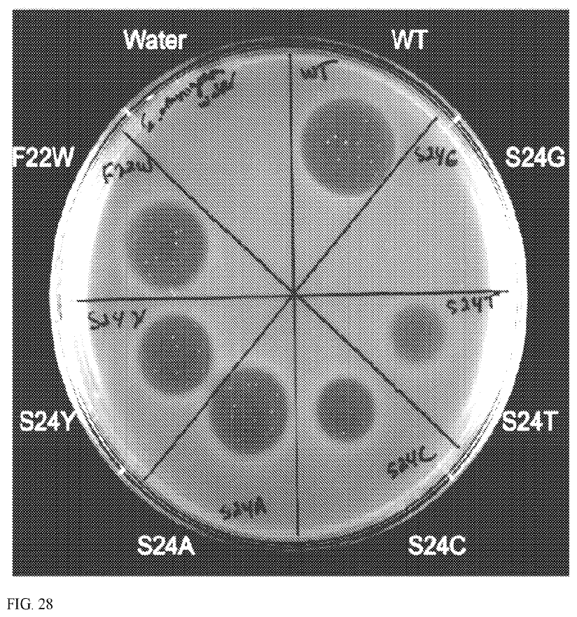

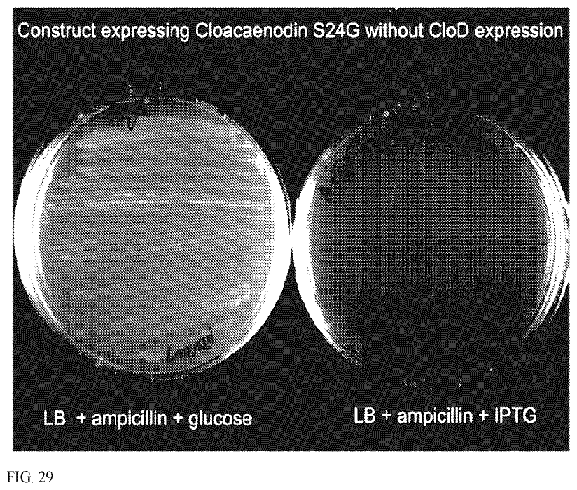

Because cloacaenodin is unique in having a C-terminal serine (Ser, S) compared to other lasso peptides that inhibit RNAP, this serine residue 24 was swapped to the more typical C-terminal glycine (Gly, G). When the S24G variant was expressed, the majority of the peptide was unthreaded in the supernatant extract, based on the later retention time as well as heating and carboxypeptidase assays (FIGS. 27A-27B). This unthreaded peptide lost its bioactivity when tested against Enterobacter amnigenus (FIG. 28). However, when expressed intracellularly without the cloD gene present, the S24G variant still inhibited Escherichia coli cell growth, suggesting that at least some fraction of the peptide remains threaded in the cytoplasm and exerts antimicrobial activity (FIG. 29). Next were made conservative substitutions to the S24 residue, cloacaenodin S24A, S24C, and S24T, each of which was similarly stable as the wild-type and had similar or decreased bioactivity compared to the wild-type (FIG. 28). Without being bound by theory, given the unthreading behavior, it can be hypothesized that swapping the S24 to a bulkier Tyr (S24Y), Phe (S24F), or Trp (S24W) residue may stabilize the lassoed shape: however, it was found that these variants still allowed unthreading. The S24Y mutant was purified by HPLC and had similar bioactivity as the wild-type (FIG. 28).

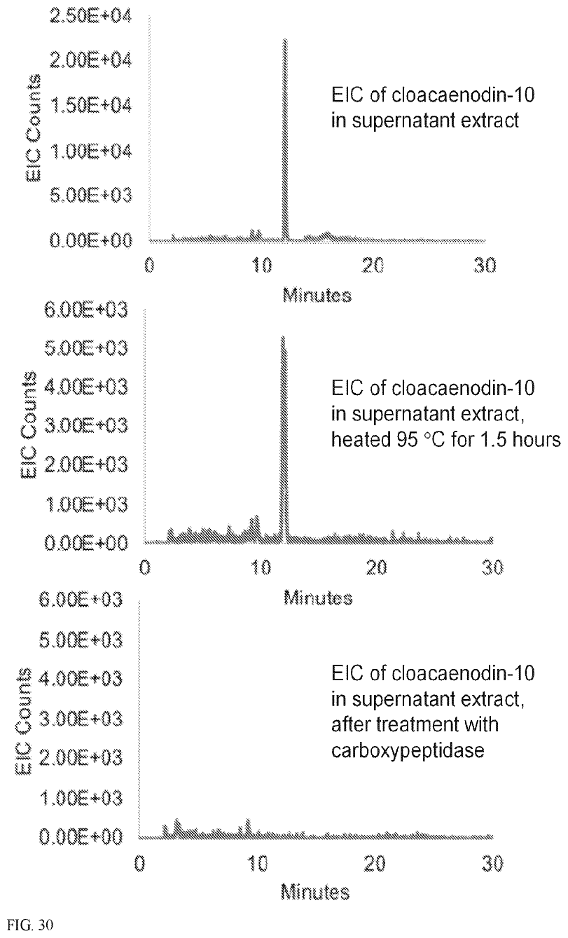

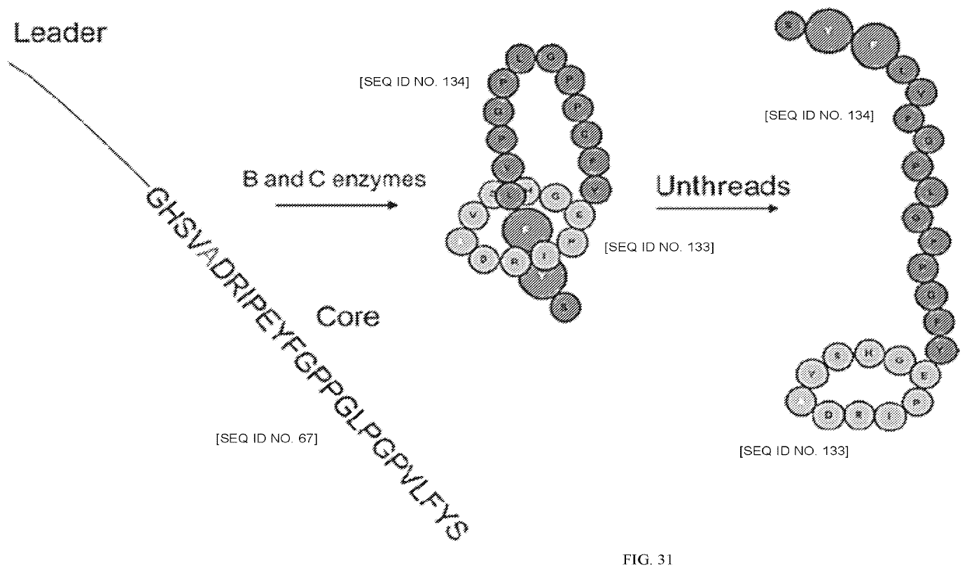

It was investigated whether the cloacaenodin biosynthetic enzymes could tolerate an increase in ring size. An increase in ring size from 9 to 10 aa (amino acids) was reported for the lasso peptide fuscanodin/fusilassin.⁶⁹ A variant of cloacaenodin with an extra alanine (Ala, A) near the middle of the ring was cloned, so that the new ring sequence was GHSVADRIPE [SEQ ID NO. 7]. A single peak corresponding to the expected mass and isotopic distribution was detected in the supernatant, eluting at about 12.1 minutes, and made at about 1% of the wild-type yield. Heating assays and carboxypeptidase assays of the extract demonstrated that this was an unthreaded peptide (FIG. 30). Without being bound by theory, it can be hypothesized that the lasso peptide is first made threaded by the B and C enzymes, but then unthreads upon being secreted into the supernatant and during the purification procedure (FIG. 31).

Although changes to isopeptide-bonded residues are generally not tolerated for lasso peptides^{70,71}, certain biosynthetic enzymes may tolerate variations at these positions.^{23,72,73} For cloacaenodin, G1A and E9D variants were not produced, as the variants could not be detected in the supernatant or the cell pellet.

A proline at position 8 in the 9-membered (9-residue) ring of the lasso peptide caulosegnin II prevented unthreading at 95° C.⁷⁴

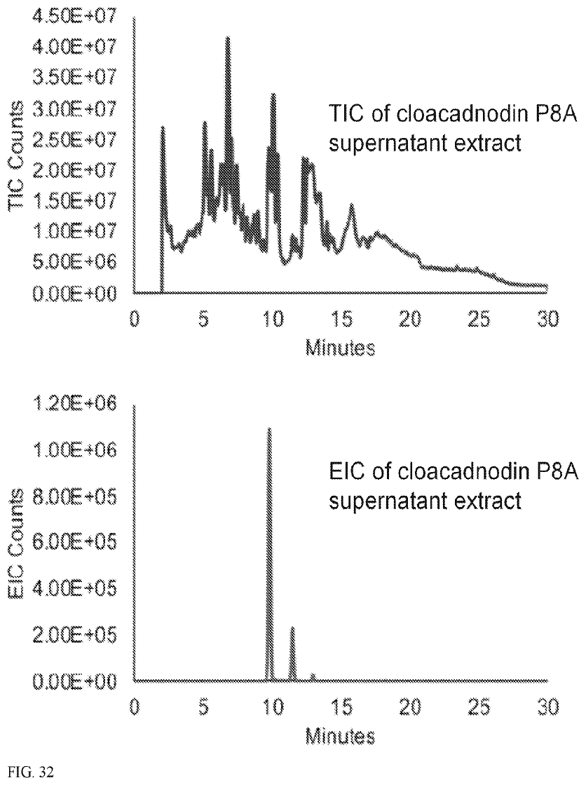

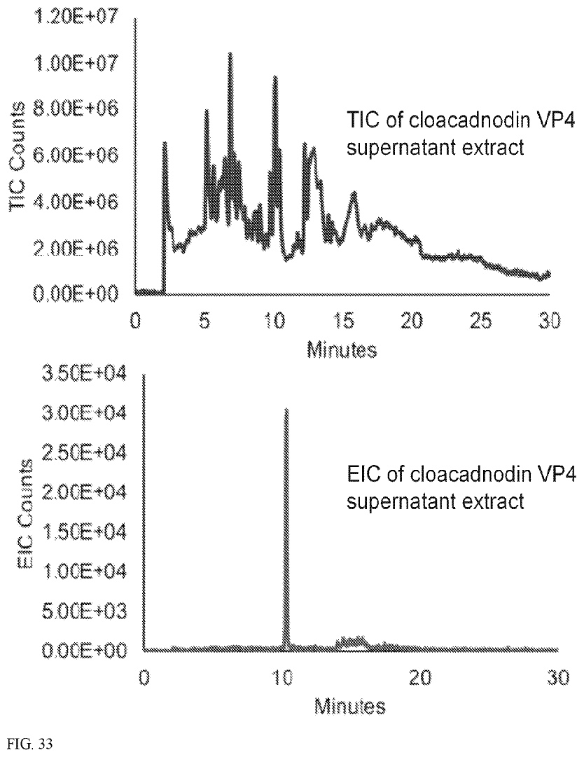

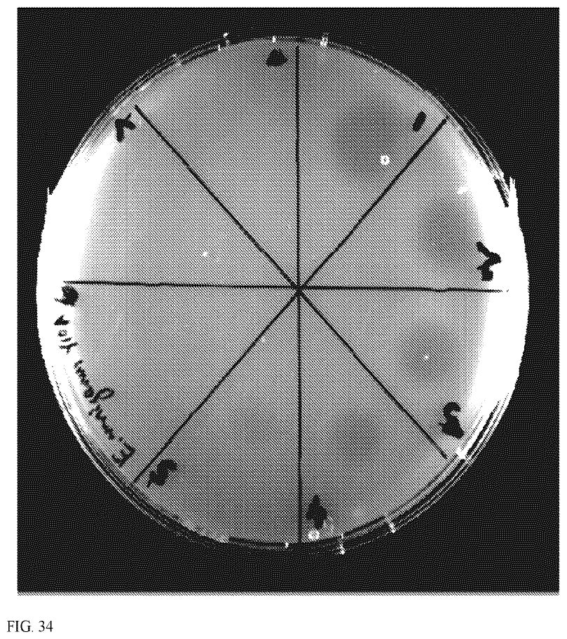

For cloacaenodin, a dramatic decrease in the ratio of threaded-to-unthreaded peptide in the extract for a cloacaenodin P8A variant (FIG. 32) was not observed. However, swapping the Val4 residue to proline (Pro, P) (V4P) appeared to favor the threaded peptide, suggesting that rigidification of the ring with proline can stabilize the lasso peptide against unthreading. However, this increased stability comes at a cost, because the V4P variant is only made at about 1% of the wild-type yield (FIG. 33).

The Tyr9 sidechain in MccJ25 (corresponding to Tyr10 in cloacaenodin) hydrogen bonds with RNA polymerase in a co-crystal structure.⁴⁰ A cloacaenodin Y10A variant was purified: despite being threaded, cloacaenodin Y10A had reduced activity against Enterobacter amnigenus (FIG. 34). When expressing this peptide in Escherichia coli without CloD present, the Escherichia coli cells could still grow, suggesting that the Y10A variant lacks activity because of decreased target binding as opposed to decreased transport into susceptible cells (FIG. 21).

### Antibiotic-Resistant Isolates are Susceptible to Cloacaenodin

Two panels from the CDC & FDA Antibiotic Resistance Isolate Bank were used: the Enterobacterales Carbapenemase Diversity (CRE) panel and the Enterobacterales Carbapenem Breakpoint (BIT) panel.⁸¹ These panels represent strains with resistance to carbapenems, which are last resort antibiotics: there is an urgent need of new treatments of infections with these strains.

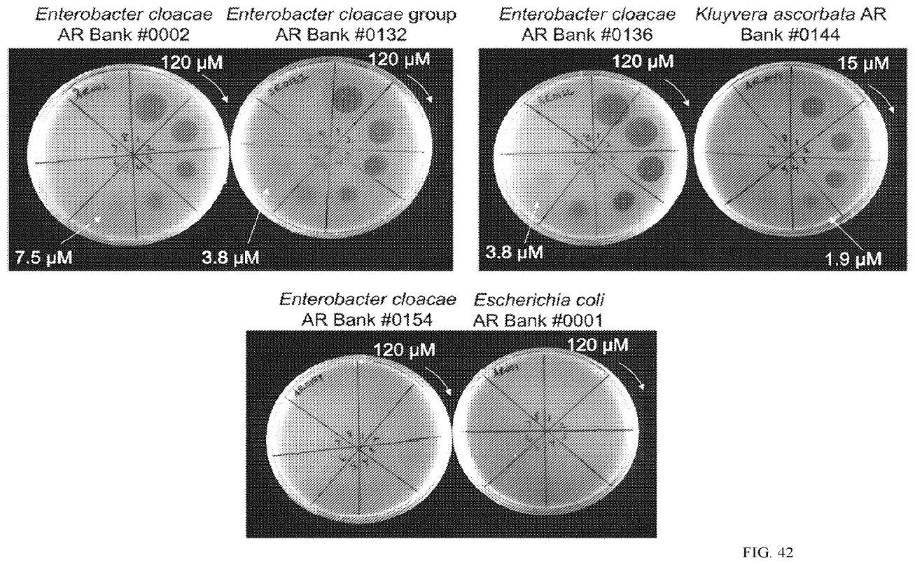

We tested 6 strains: 4 Enterobacter strains, 1 Kluyvera strain, and 1 Escherichia coli strain. Cloacaenodin has single digit micromolar activity against the Kluyvera strain and three (3) of the Enterobacter strains (FIG. 42).

Peptides from Other Strains: Heterologous Production and Activity

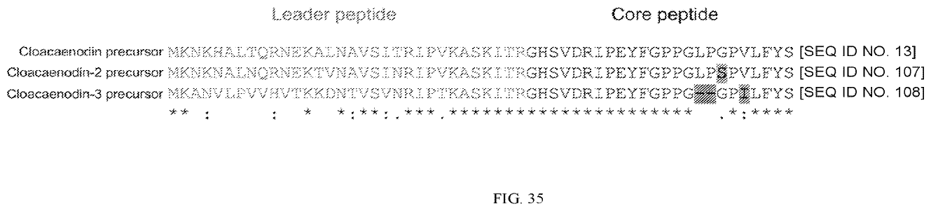

Biosynthetic gene clusters (BGCs) encoding cloacaenodin-like peptides with similarities to cloacaenodin were found in other species of bacteria, including other Enterobacter strains and one Citrobacter strain.⁷⁹ A number of the core peptides are identical in sequence to cloacaenodin, while some have deviations. A peptide encoded by Enterobacter hormaechei subsp. xiangfangensis strain 120070, a strain isolated from a human blood sample in China, is a G18S variant of cloacaenodin. In Citrobacter sp. CtB7.12, a strain isolated from the gut microbiome of a termite in Mexico,⁸⁰ the encoded core peptide has a deletion (1) of the L16 and P17 residues, and a V20I variation, compared to cloacaenodin. Thus, the overall lasso peptide is 22 aa (amino acid residues) instead of the 24 aa (amino acid residues) of cloacaenodin, with the deleted amino acids reducing the loop size by two (2) aa (amino acid residues). The cloacaenodin G18S variant was named cloacaenodin-2, and the cloacaenodin ΔL16 ΔP17 V20I variant was named cloacaenodin-3. The core peptide sequences of cloacaenodin-2 and cloacaenodin-3 are GHSVDRIPEYFGPPGLPSPVLFYS [SEQ ID NO. 105] and GHSVDRIPEYFGPPGGPILFYS [SEQ ID NO. 106], respectively, where the underlined residues are changes from wild-type cloacaenodin (FIG. 35).

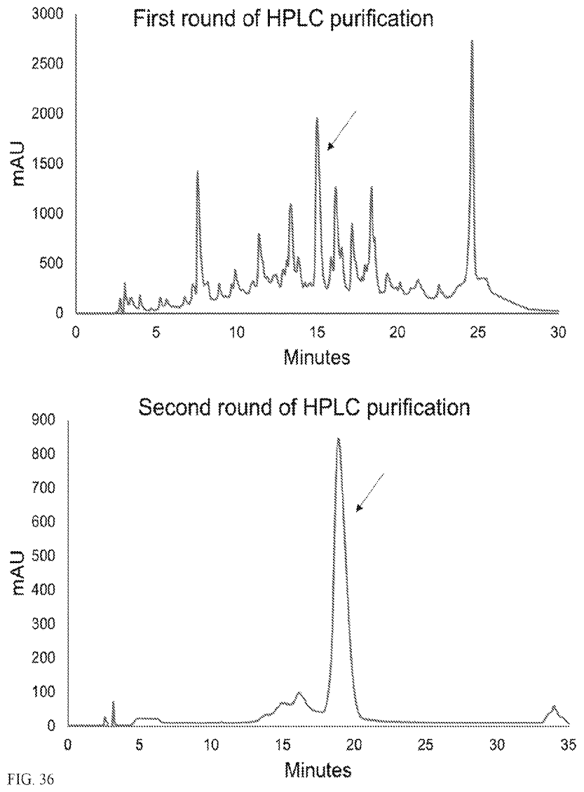

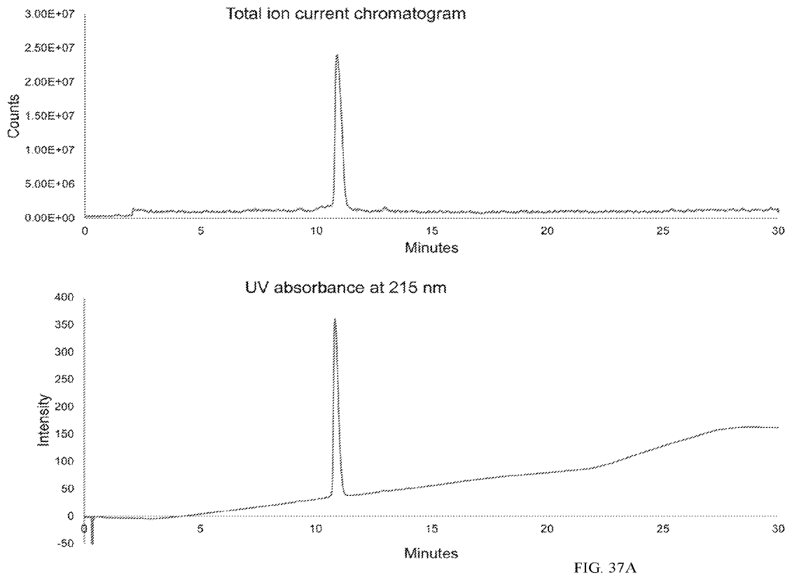

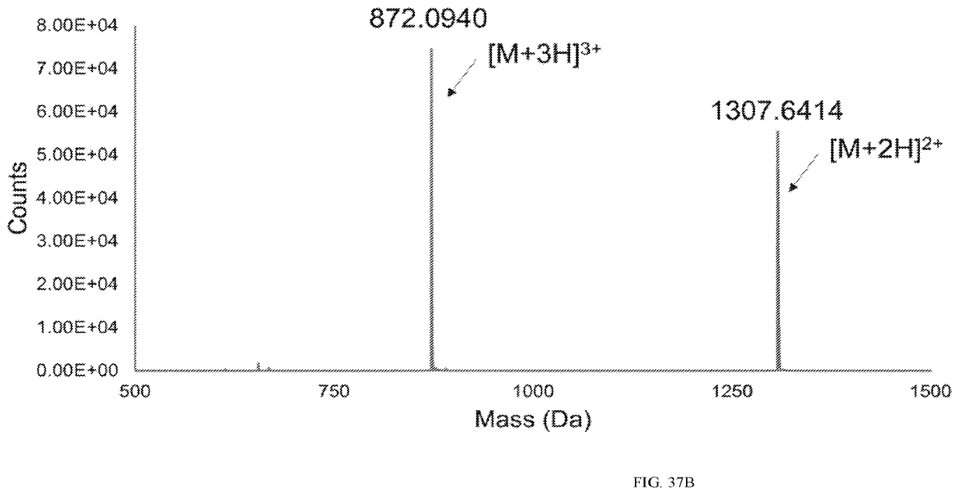

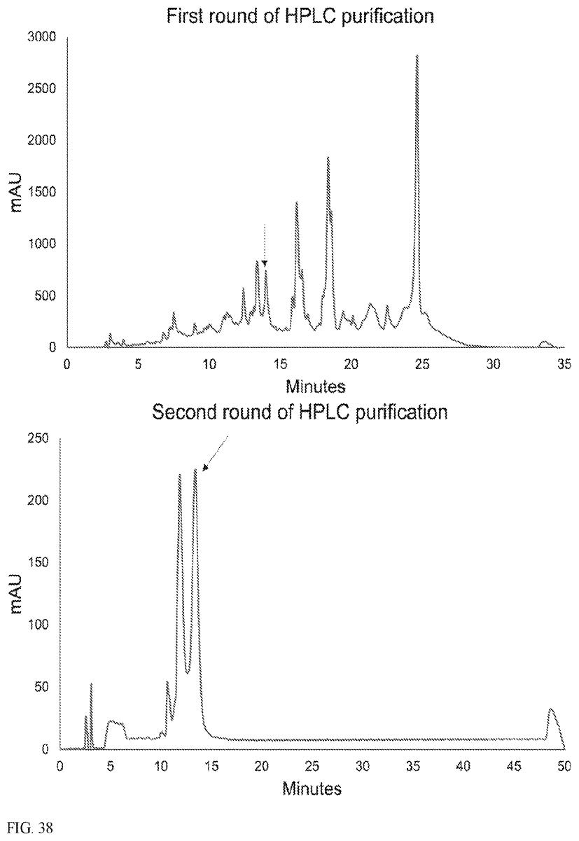

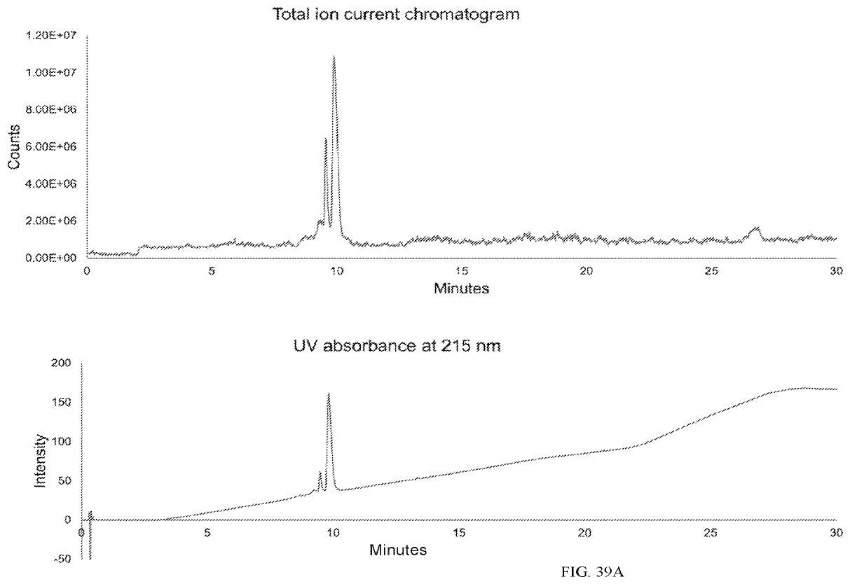

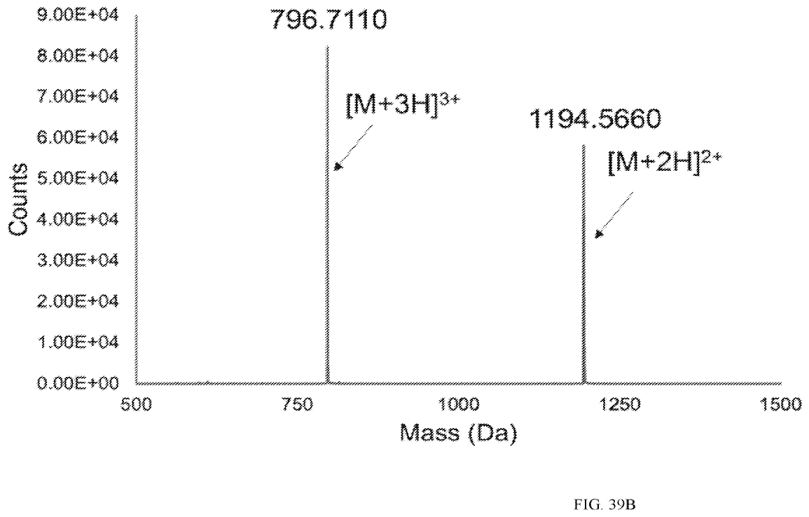

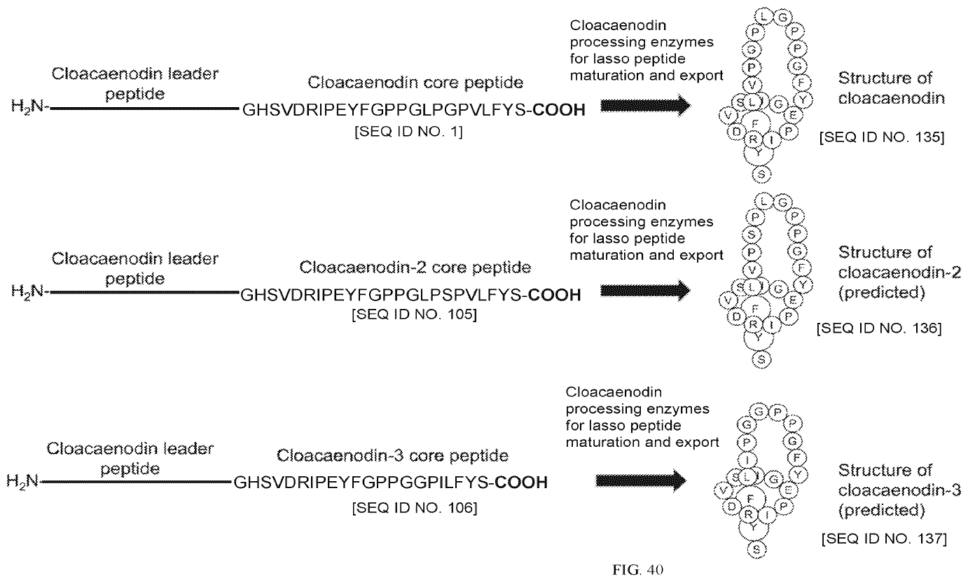

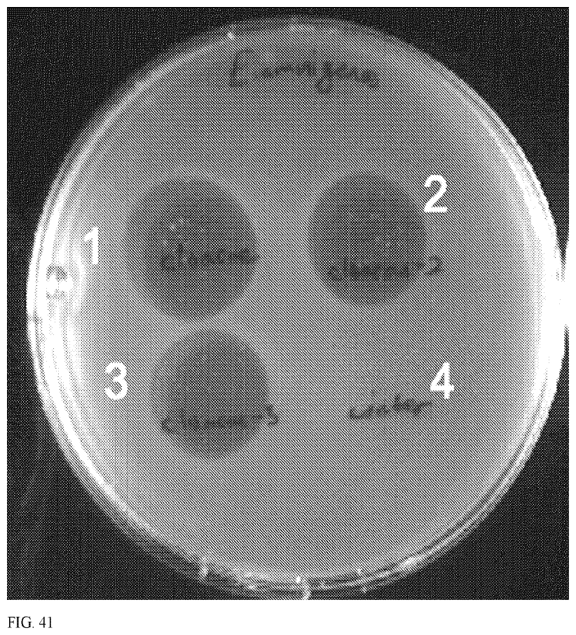

By making mutations to the cloacaenodin expression plasmid in the core peptide region of cloA, we successfully heterologously expressed these two lasso peptides, cloacaenodin-2 and cloacaenodin-3. While the B, C, and D protein sequence for the various lasso peptides differ from each other⁷⁹ (and are most dissimilar for cloacaenodin-3, from Citrobacter), the two peptides were still produced using this heterologous expression system using the biosynthetic machinery of cloacaenodin (FIGS. 36-39B). These data offer insight into the substrate tolerance of the cloacaenodin biosynthetic machinery, particularly that the loop can be shortened by 2 aa (amino acid residues) and still be correctly processed (FIG. 40).

We tested purified cloacaenodin-2 and cloacaenodin-3 against Enterobacter amnigenus, a strain susceptible to wild-type cloacaenodin. We found that these peptides cloacaenodin-2 and cloacaenodin-3 showed similar activity against Enterobacter amnigenus as did cloacaenodin (FIG. 41).

### Methods

Detailed experimental methods and materials used, including details on cloacaenodin BGC identification, cloacaenodin heterologous expression, NMR analysis, antimicrobial assays, and protease assays, are provided in the following.

### Materials

For cloning, E. coli XL-1 blue cells were used, while for peptide expression, E. coli BL-21 cells were used. Q5 DNA polymerase from New England Biolabs (NEB) was used for polymerase chain reaction (PCR), and all restriction enzymes and T4 DNA ligase used were purchased from NEB. Plasmids were purified using mini-prep spin columns from QIAGEN. Deoxyribonucleic acid (DNA) fragments for molecular cloning were gel extracted using Zymoclean Gel DNA recovery kits from Zymo. Commercial strains for testing cloacaenodin activity were purchased from Leibniz Institute DSMZ or American Type Culture Collection and are listed in Table 6. Primers and gBlocks were ordered from Integrated DNA Technologies and cloned plasmids were sequence confirmed with Genewiz (now Azenta) before expression. All primer sequences are listed in Table 10 and resulting plasmids are listed in Table 11. After expression, cultures were spun down with an Avanti J26S XP centrifuge from Beckman Coulter. For supernatant purification, HyperSep C8 columns from Thermo Fisher with a 6 mL column volume were used. Methanol extracts were rotovapped with a Buchi Rotovapor R-210. LC-MS analysis and LC-MS/MS analysis were done using an Agilent 6530 QTOF connected to an Agilent 1260 LC, with all analysis done using electrospray ionization with the instrument in positive ion mode. LC-MS data were visualized using Agilent MassHunter software, and MS/MS data were visualized using mMass software. The column used for LC-MS analysis was an Agilent Zorbax C18 column, with size 2.1 mm by 50 mm and 3.5 μm particle size. For HPLC, an Agilent 1200 series HPLC was used, with extracts purified using an Agilent Zorbax C18 column, with size 9.4 mm by 250 mm and 5 μm particle size. HPLC-grade solvents were used for LC-MS and HPLC, with acetonitrile purchased from Sigma-Aldrich. Collected HPLC fractions were lyophilized using a Labconco FreeZone 4.5.

TABLE 10
Primer sequences used in this study. The sequences shown are from the 5′ end to 3′ end.

| Primer | | |
|---|---|---|
| Description | Sequence | |
| Cloacae | CGATCGTGAATTCATTAAAGAGGAGAAATTAACTATGGATG | [SEQ ID NO. 68] |
| forward | | |
| Cloacae Y23W | CGATGCAAGCTTTTAGCTCCAAAACAGGACTG | [SEQ ID NO. 69] |
| reverse | | |
| Cloacae F22W | CGATGCAAGCTTTTAGCTATACCACAGGACTGG | [SEQ ID NO. 70] |
| reverse | | |
| Cloacae S24G | TGACGAAGCTTTTAGCCATAAAACAGGACT | [SEQ ID NO. 71] |
| reverse | | |
| Cloacae S24T | ACAGAAAAGCTTTTAGGTATAAAACAGGACTGG | [SEQ ID NO. 72] |
| reverse | | |
| Cloacae S24C | AAGCAAAAGCTTTTAGCAATAAAACAGGACTG | [SEQ ID NO. 73] |
| reverse | | |
| Cloacae S24A | AAGCAAAAGCTTTTAGGCATAAAACAGGACTGGA | [SEQ ID NO. 74] |
| reverse | | |
| Cloacae S24Y | ACAGAAAAGCTTTTAATAATAAAACAGGACTGGAC | [SEQ ID NO. 75] |
| reverse | | |
| Cloacae S24W | ACAGAAAAGCTTTTACCAATAAAACAGGACTGG | [SEQ ID NO. 76] |
| reverse | | |
| Cloacae S24F | ACAGAAAAGCTTTTAAAAATAAAACAGGACTGG | [SEQ ID NO. 77] |
| reverse | | |
| Cloacae | GCTAATTAAGCTTTTAGCTATAAAACAGGAC | [SEQ ID NO. 78] |
| reverse | | |
| Cloacae BGC D | ACGATGGATCCGTCGTGAGG | [SEQ ID NO. 79] |
| removal forward | | |
| Cloacae BGC D | GGCATACCATGGTTAAACAAACTCAAAAAATTCA | [SEQ ID NO. 80] |
| removal reverse | TGCCTTG | |
| Cloacae GIA | ACGCTATGGGCACGTGTAATTTTGCTCGCTTTC | [SEQ ID NO. 81] |
| internal reverse | | |
| Cloacae G1A | CGTGCCCATAGCGTGGATCGTATTC | [SEQ ID NO. 82] |
| internal forward | | |
| Cloacae E9D | GTAGTCCGGAATACGATCCACGC | [SEQ ID NO. 83] |
| internal reverse | | |
| Cloacae E9D | GGATCGTATTCCGGACTACTTTGGCCCTCCGG | [SEQ ID NO. 84] |
| internal forward | | |
| Cloacae Y10A | GAGGGCCAAAGGCCTCCGGAATACGATCCAC | [SEQ ID NO. 85] |
| internal reverse | | |
| Cloacae Y10A | GGAGGCCTTTGGCCCTCCGG | [SEQ ID NO. 86] |
| internal forward | | |
| Cloacae P8A | CCGCAATACGATCCACGCTATGG | [SEQ ID NO. 87] |
| internal reverse | | |
| Cloacae P8A | CGTGGATCGTATTGCGGAGTACTTTGGCCCTC | [SEQ ID NO. 88] |
| internal forward | | |
| Cloacae V4P | CGATCCGGGCTATGGCCACGTGTAATTTTG | [SEQ ID NO. 89] |
| internal reverse | | |
| Cloacae V4P | CCATAGCCCGGATCGTATTCCGGAGTAC | [SEQ ID NO. 90] |
| internal forward | | |
| Cloacae | AATACGATCCGCCACGCTATGGCCACG | [SEQ ID NO. 91] |
| 10-member (10- | | |
| residue) ring | | |
| internal reverse | | |
| Cloacae | CGTGGCGGATCGTATTCCGGAGTACT | [SEQ ID NO. 92] |
| 10-member (10- | | |
| residue) ring | | |
| internal forward | | |

TABLE 11
Plasmids used.

| Plasmid Name | Description | Host vector |
|---|---|---|
| pAK2 | Refactored cloacaenodin BGC | pQE-80L |
| pMP1 | Cloacaenodin Y23W | pAK2 |
| pMP2 | Cloacaenodin F22W | pAK2 |
| pMP3 | Cloacaenodin BGC | pAK2 |
| | without D gene | |
| pMP4 | Cloacaenodin S24G | pAK2 |
| pMP5 | Cloacaenodin S24T | pAK2 |
| pMP6 | Cloacaenodin S24C | pAK2 |
| pMP7 | Cloacaenodin S24A | pAK2 |
| pMP8 | Cloacaenodin S24Y | pAK2 |
| pMP9 | Cloacaenodin S24W | pAK2 |
| pMP10 | Cloacaenodin S24F | pAK2 |
| pMP11 | Cloacaenodin G1A | pAK2 |
| pMP12 | Cloacaenodin E9D | pAK2 |
| pMP14 | Cloacaenodin Y10A | pAK2 |
| pDVC060 | Cloacaenodin P8A | pAK2 |
| pDVC061 | Cloacaenodin V4P | pAK2 |
| pDVC062 | Cloacaenodin 10-membered ring | pAK2 |
| pDVC066 | Cloacaenodin Y10A in cloacaenodin | pMP3 |
| | BGC without D gene | |
| pDVC067 | Cloacaenodin S24G in cloacaenodin | pMP3 |
| | BGC without D gene | |

### Identification of Gene Cluster and Bioinformatic Search of Cloacaenodin-Like Gene Clusters

A precursor-centric genome mining algorithm was used²⁶, with searching for precursors with the tyrosine (Tyr, Y) after the expected ring and penultimate Tyr. Upon identification of the cloacaenodin BGC in Enterobacter hormaechei strain LB3, a BlastP search was conducted on the amino acid sequence for CloA using the following default parameters: standard database search set, non-redundant protein sequences database, blastp algorithm, 100 maximum target sequences, parameters automatically adjusted for short input sequences, expected threshold of 0.05, word size of 6, 0 max matches in a query range, BLOSUM 62 matrix, gap costs of existence: 11 and extension: 1, conditional compositional score matrix adjustment, low complexity regions filtered. We then manually searched nearby the identified A genes to confirm the presence of the B, C, and D genes. To search for the total number of assembled Enterobacter cloacae and Enterobacter hormaechei genomes, an assembly search was conducted on NCBI with “Enterobacter cloacae” or “Enterobacter hormaechei” as the search query on Aug. 25, 2022, and the total number of “Latest GenBank” assemblies was recorded. To compare the amino acid sequences of the CloA, CloB, CloC, and CloD-like proteins, the multiple sequence alignment tool was used through Clustal Omega on the web server.⁷⁶

### Cloning and Plasmid Construction

The BGC of cloacaenodin (consisting of the ABCD architecture) was codon-optimized for E. coli using DNAWorks.⁷⁷ The codon-optimized sequence was used for the gene refactoring into pQE-80. This refactored gene cluster contains the A precursor under the control of the isopropyl-β-D-thiogalactopyranoside (IPTG)-inducible T5 promoter in pQE-80, with the other genes of the BGC (B, C, and D) placed under the natural constitutive mcjBCD promoter of the microcin J25 gene cluster. Briefly, the cloA gene was cloned following the T5 promoter and ribosome binding site (RBS) of pQE-80 using EcoRI and HindIII restriction sites. This was assembled with primers listed in Table 12. gBlocks encoding the codon-optimized cloBCD genes were amplified via overlap PCR with a preceding pmcjBCD promoter. These gBlock sequences are listed in Table 13. The resulting purified PCR product was then cloned the plasmid containing cloA using the NheI and NcoI restriction sites. This resulted in formation of the pAK2 plasmid (pT5-cloA pmcjBCD-cloBCD), which was verified by sequencing from Genewiz (now Azenta).

TABLE 12
Primers used to assemble cloA gene in pAK2.
The sequences shown are from the 5′ to the
3′ end.

| Primer | Sequence | |
|---|---|---|
| 1F | CACACAGAATTCATTAAAGAGGAGAA | [SEQ ID |
| | ATTAACTATGGATGTGATGA | NO. 93] |
| 2R | GGTCAGCGCGTGTTTATTCTTCATCA | [SEQ ID |
| | CATCCATAGTTAATTTCTCCT | NO. 94] |
| 3F | AGAATAAACACGCGCTGACCCAGCGT | [SEQ ID |
| | AATGAAAAAGCGCTGAATGCG | NO. 95] |
| 4R | TTCACCGGTATACGGGTGATGCTCAC | [SEQ ID |
| | CGCATTCAGCGCTTTTTCATT | NO. 96] |
| 5F | CATCACCCGTATACCGGTGAAAGCGA | [SEQ ID |
| | GCAAAATTACACGTGGCCATA | NO. 97] |
| 6R | AAGTACTCCGGAATACGATCCACGCT | [SEQ ID |
| | ATGGCCACGTGTAATTTTGCT | NO. 98] |
| 7F | GTGGATCGTATTCCGGAGTACTTTGG | [SEQ ID |
| | CCCTCCGGGCCTGCCGGGTCC | NO. 99] |
| 8F | CTAATTAAGCTTTTAGCTATAAAACA | [SEQ ID |
| | GGACTGGACCCGGCAGGCCC | NO. 100] |

TABLE 13
gBlock sequences used to assemble pAK2.
The sequences shown are from the 5′ to the 3′ end.

| gBlock | Sequence | |
|---|---|---|
| 1 | GCGTTTTTTATTGGTGAGAATCCAAGCTAGCCATCAATTAA | [SEQ ID NO. 101] |
| | GAAAAAAATTTAGCTTGTAGATAAATTCAGAAGTTTTATTAT | |
| | TCCAATTGAGTGTAAAGGCATAACTACAGGAGGGAGTGTG | |
| | CAAAATGCTGATTACGCCGGATACCCTGAAAATTACCTGCT | |
| | TTAACAAGGATCTGGTGGTGCTTGACTTACTGAAAGATGAA | |
| | TATACCGTGCTGGAAAATACCAGCGATAGCTTCCTGACCTT | |
| | TCAGATTCCGAACCCTGACGTAGAGGCGTTTCTGAATGGC | |
| | GAAAAAAACTGCCGTTATCTGCAGGCGGATAGCCACAATG | |
| | ATCCCGAATCTTTTCTGGAAGTGCGTTGGATGAAACCGCA | |
| | GGTGCGTATTAAACATTCTAGCGCCCTGGCTCGTATCTCG | |
| | GCGCTGCGTGCGATGCTGTATTGTCGGCATGGCCTGAATC | |
| | ATGGGGGCTTGATTGCGCTGAAACGTCAGTTTTGCGAAAG | |
| | CCAGACCATTGGCTGCGCGACCAAAATCACTCCGGAAGTG | |
| | GACATTCGTATCAAGAAAGAAATTGCGGCGTTGAATAGCAT | |
| | TTTTGATCTGATTGATCACGATAATCCGTGCCTGGTCTATT | |
| | CGTGGGCGCTTAAACAGCGCCTGATTAGCCGTAAAATTGG | |
| | CGCAACCCTGGTGATTGGCGTGCGTACCCGTCCGTTTTAT | |
| | AGCCATGCGTGGGTGGAGGTGGATAGCACCGTGGTGAGC | |
| | GATGATCCGGACCTGAGAAAGAAAACGAGCGTCATACTGG | |
| | AGTGCTGACCATGCTGATTGTGCCAGTGATGCTGTCCGAA | |
| | AGCGTGGCAGTGCCGGCTCATTTTACCGCTGTGCGTACCG | |
| | GGATGTACCTGATTTATCTGGACGGGGGCTGGAGCTATCG | |
| | GGAAGATGACGGAAAACTGTGCATTTTTAGGGGCTGGGCG | |
| | ATTACCGGCAATGGCGAAGTGTTATCGTTGAGCGAGACCG | |
| | CACCTGATGTGCTGGAGAAAGAGCTTAAAAGCGCCGATGG | |
| | CTATTTTTGCTTTATTGTTATAGAAAATGATGTGATACAAGT | |
| | AATGAAGAGCCTGTATCGTTATTGCGATGTGTACTACGGCA | |
| | AGACTGCGTTTGGCGCTATTATTGGCACCGGCTTGAAAGA | |
| | TGTGGCGCGGATTAGCGGGACCAAAACCATTACTCGGGAC | |
| | TTTATTCGTAGCTTCATACGGGATAGCTTTCAGGCGGCGTT | |
| | TCTGAGCCCGCTGCATAATACCGGAAAGGCGTACTCCGGCT | |
| | GCTTGACCCGGATTGACCGTGAAAATATCCATACCGAAGCGT | |
| | TTGTGAATCTGACCCCTCTGAAGAAAAACACCCTGGACGT | |
| | GATTATTACGATTGTGAAAAAACTGCCAGCCGATCTGCCGC | |
| | TGGCGTTATATCTGTCCGGCGGATTGGATAGCAGCATCGTGT | |
| | TCTTCTGCCTGCTGGCTGCGGATAAACCGTTTA | |
| 2 | TGCTGGCTGCGGATAAACCGTTTACCGTGTTTCATTGCTTA | [SEQ ID NO. 102] |
| | CCGTTCAGCTTTGAAACCGACGCAGAACGGGGGACGCG | |
| | CATCGTCTGTGCCGTAAGTATAACATTACCCTGACCGAGCT | |
| | GAAACCTGGCTATACCACCGATCGCCAAATTAGGAATCGT | |
| | GTAAATCATCCGAGCGATTGTCCGGTGGTGAATACCTTTTG | |
| | GCTGAATGCGAGTGACGGCCTGACACAAGATATGAGCGGGT | |
| | GGTTCTGCTTGGACGGTCATGGCGGCGACAGCGTGTTTATTC | |
| | AGAGCCCGAGCGTGCGCATCATTCGTGACTTAATTCGCGTGG | |
| | GCAAACCGATTCAAGCGTTGCGGACCGCATTCCGTCTTGCAG | |
| | CGTTGAAAAGCCGCTCTTTACACAGCGTACTGGCGAGCGCA | |
| | TACAAAACCGAACCGGCGAAACGTACCCATGTTCATCCCC | |
| | TTCTGGTGGGCCATATTCCAGGCACCGCATGGTATGAATA | |
| | CTTGGAAGAACTTATCCGTATTAGCGAAAGCACAGTGTCAA | |
| | TTTTTCCGGATGGACCGGCGGTGAAATTTAGTCCTCTTATT | |
| | AGCCTGAGCGTGATTCGTTCACGTCTGGGGGTGCGTTAGG | |
| | AGGATAACATACGTGCTGGGCATGATCGTGGGGGGATCCG | |
| | TCGTGAGGCGCTGACCCGTTTTAAAGATCCGGTGTTCATA | |
| | AAGAAAACTAAGCGTTCCAGCAGCCAATTGATTTTTACAAT | |
| | TATTCGGCAGTATGAGGACGAACTGATTGCGTTCGTGGTC | |
| | CGTTATGGCACCTATATTCTGGACAACCCTGAAGCGATGAT | |
| | TGAGGAAATTCGTTATAATGCTCATGTGGAATTAAACGGCA | |
| | GCCTGCCGGAAATTTTGTCCCTGATTAAACTGATGGTCTTT | |
| | AGCCAGAAGATGGAAGCTGATTTTTTCGACGCGGAGTCAA | |
| | GGCATGAATTTTTTGAGTTTGTTTAAAACCGTGAGCCGTGC | |
| | GACCGGCACCCGTCATGTGGGCAAAATTACGTTCATTATCC | |
| | TGCTGAGCGTGTTAAGCAGCGTGCTGATCATAATTTCTCCGGT | |
| | CCTGATTAGTCGTACAACCGGCTACCTGATGAGCGATACC | |
| | GCGGATAGCTTTCGTGTGATATTTACCTTGGGTGCGGTGT | |
| | ATGTGTTTGCCATTTCAGCGCAAAAGATAATTGCTTTTTGG | |
| | GCGACCTACCTGCAGAGCGCCCTGCGCGTGGAATGTATTG | |
| | CGAGCATAAGCGAGTTTTTCCTTAAGAAACTGTATTATAGC | |
| | GGTAGCCAGAAGGACAACGCTGGTGGAATTAGCCAGCAAC | |
| | TTAATCAAGCCACCAATGACATTTACATAATCATCCGTGAAC | |
| | TGACCTTCGGCCTGTTACCACCGCTGATTCAGCTGTTTATTGC | |
| | CCTGTGGACCATTTTAGCGAGCGATGATGTGATTGTGGCGT | |
| | GCAT | |
| 3 | TGATGTGATTGTGGCGTGCATGTTTCTGATGTTTGCGATTAG | [SEQ ID NO. 103] |
| | CTTTGTGTTTACCAATTATGTGTATACCCATAAAATCGTGGC | |
| | GGCTCGTCAGGCGCTGATGGACAGCAGCCGTAAAACCTAC | |
| | ACCTTATTATCCGATTCGGTCCGTAATATTCCGGTGGTGCG | |
| | GGTGTTTAACGGCTTTGGCGTGTTTATGAAGCGTTTCAACGGC | |
| | CAGCTGACAGAGGATGCGCGTACCCAGCGTAGCTTTTGGAA | |
| | AGTTAGCTTCCATGCGCAGCTGGTGTCGGCGTTACTGCAGA | |
| | TAATCTTTTTTGGCTCCGCGTTCTTTTATACTCTGTATGGCG | |
| | CGAGCACCGGTCAGGTGACCATTGAGCATTTCATTCTGGTG | |
| | AGCAGCTACATTCTGATTTTGAGTGCCCCCTTGGAAAATCTG | |
| | GGCCAGTCCTATATCAATTTTCGTCAGAGTGCTCATGCGTTT | |
| | AGCGCATTTAGCGGCGGTTGGCTGGAAGAACCGGTGGATC | |
| | GTGATGGCAATCTGCGCCGTCCCCAAAACGGGGATATTACT | |
| | CTGAGCGGCGTGTCATTTCGTTACCCGGAAAGCCCGAGCTG | |
| | CACACTGCATGACGTGAATTTACACTTCCGCACGGGCGAGT | |
| | TCATAACCATTACCGGCAAGTCCGGTTCAGGCAAAAGCACC | |
| | CTGTTGAAAATCCTGGCACGTCAGCTTCCGCTGACCATAGG | |
| | GAACTATACACTGGGAGGCTGCCCTAATGAAGAACTGCCTG | |
| | AGCAAGTGTTCTACGAAAACATTGCATATGTGAGCCAAGAA | |
| | GAATATGTCTTTATGGATAGCGTGGCGTATAACTTACAGATC | |
| | GCGAATCCGGAAGCGACTCGTGAAGAGATGATCTCGGCCC | |
| | TGGCGAAAAGCGGACTGCAAATTGGACATCTGCAAGGCGAA | |
| | GCGCTGCTGGATTTGAACCTGGCCAATGAAGGCACCAACAT | |
| | TTCCGGAGGTCAACGCCAACGGCTGAGCTTGGCGCGTTTGT | |
| | TTTTGCGTAACCCGGCAGTGATACTGTTGGACGAGATTACC | |
| | TCTAGCCTGGATCTTATATCTGAAACCCACGCAATTAACGCG | |
| | ATTTTGTCAGACTTTCCGACCGCGACCATTATTTCGATCTCG | |
| | CATCGTATAAGCACCTTTAACTATAGCGATCGTATTATTGTG | |
| | ATGCATAACGGTCGGGTTAGCGATGCAGGCCCCCTGAACG | |
| | AACTGCTGCAACGTAACAACTTCATAGCTCAACTGCACCGTA | |
| | GCGAAGAAAGTCCACCTGTGAAAGCGCCATGGGCAAATATTAT | |
| | ACGCAAGGCGAC | |

It was found after assembling the gBlocks that a stop codon was missing on the cloned cloD gene; we corrected this with the following primer sequence:

[SEQ ID NO. 104]
5′-GCGTATAATATTTGCCCATGGTTACGCTTTCACAGGTGGACTTT
CTTCGC-3′.

Cloacaenodin variants were constructed using site-directed mutagenesis. Mutant precursor genes were amplified from the wild-type precursor in pAK2 using mutagenic primers. Following PCR amplification of the mutated precursor gene, the purified PCR product was digested and ligated into pAK2 with the EcoRI and HindIII restriction sites. For the pMP3 plasmid for deletion of the cloD gene, the D gene was first disrupted by digestion of pAK2 with BamHI and NcoI, which removed the entire cloD gene and the C-terminal portion of the cloC gene. The digested plasmid was then ligated with an insert that restored the C-terminal portion of the cloC gene. All mutants were sequence confirmed by Genewiz (now Azenta) before use.

### Expression and Purification of Cloacaenodin and Mutants

pAK2 was transformed via electroporation into Escherichia coli BL-21 cells before plating on an LB agar plate supplemented with 100 μg/mL of ampicillin. Following overnight incubation of the plate at 37° C., a single colony was then used to inoculate 5 mL of LB broth supplemented with 100 μg/mL of ampicillin. This culture was then grown at 37° C. with 250 rpm shaking overnight. The following day, the OD₆₀₀ of the overnight culture was measured, and this was diluted to an OD₆₀₀ of 0.02 in 500 mL M9 minimal media in a 2 L flask. The M9 minimal media consisted of M9 salts, 0.2% glucose, 1 mM MgSO₄, 0.00005 wt % thiamine, and the 20 amino acids each at a concentration of 40 mg/L. 100 μg/mL of ampicillin was also added to the culture for plasmid selection. Following inoculation with the overnight culture, the 500 mL cultures were allowed to grow at 37° C. with shaking at 250 rpm. Once the OD₆₀₀ of these cultures reached approximately 0.2 (˜3-4 hours), 1 mM of IPTG was added to the cultures to induce expression of cloacaenodin. The culture was allowed to grow overnight at room temperature, with shaking at 250 rpm.

After expression, the cells and supernatant were separated by centrifugation at 4000×g for 15 minutes at 4° C. To purify the supernatant, the supernatant was extracted through a 6 mL Strata C8 column through the use of a vacuum chamber. The column was activated with 6 mL of 100% methanol before being washed with 12 mL of deionized (DI) water. The supernatant was then added to the column and allowed to flow through. After the supernatant was flowed through, the column was washed with 12 mL of DI water, and then 6 mL of 100% methanol was added to elute the extract. The methanol was then dried with a rotovap, and following this, 1 mL of DI water per liter of expression was used to resuspend the dried extract. The extract was then spun down further on a tabletop centrifuge before injection on LC-MS.

The LC-MS was operated at 0.5 mL/min of a water/acetonitrile gradient with the addition of 0.1% formic acid. From 0-1 min, 90% water/10% acetonitrile flowed through the column, followed by a linear gradient from 90% water/10% acetonitrile to 50% water/50% acetonitrile from 1-20 minutes, followed by a linear gradient from 50% water/50% acetonitrile to 10% water/90% acetonitrile from 20-25 minutes.

Via LC-MS, cloacaenodin was detected in the supernatant extract. The supernatant extract was used for RP-HPLC purification of cloacaenodin. 20-60 μL of the supernatant extract was injected onto a C18 semi-preparative column. The HPLC was operated at 4 mL/min of a water/acetonitrile gradient with the addition of 0.1% trifluoroacetic acid. From 0-1 min, 90% water/10% acetonitrile flowed through the column, followed by a linear gradient from 90% water/10% acetonitrile to 50% water/50% acetonitrile from 1-20 min, followed by a linear gradient from 50% water/50% acetonitrile to 10% water/90% acetonitrile from 25-29 minutes. Multiple peaks on the chromatogram were collected and checked via LC-MS for cloacaenodin's expected mass. The prominent peak with a retention time of 15.1 minutes matched the expected mass of cloacaenodin. This peak was then collected from the HPLC and frozen at −80° C. within minutes of collection. This was done to minimize unthreading of cloacaenodin in solution in the HPLC collection vial. After freezing fully, the frozen sample was then lyophilized and re-suspended in pure water. To determine the concentration of purified cloacaenodin, a NanoDrop spectrophotometer was used to measure the absorbance at 280 nm. From the amino acid sequence of cloacaenodin, an extinction coefficient of 2560 cm⁻¹ M⁻¹ was calculated and used for the NanoDrop measurements.⁷⁸

Cloacaenodin variants were expressed in the same way as the wild-type and purified from the supernatant. The production levels of each variant were judged via HPLC relative to the wild-type. Variants with identifiable peaks on the HPLC and appreciable production levels were purified for further assays.

For the Y10A cloacaenodin variant, a second round of HPLC was required to further purify the peptide with a flatter gradient. For the second run, the HPLC was operated at 4 mL/min of a water/acetonitrile gradient with the addition of 0.1% trifluoroacetic acid. From 0-1 min, 90% water/10% acetonitrile flowed through the column, followed by a linear gradient from 90% water/10% acetonitrile to 75% water/25% acetonitrile from 1-3 minutes, followed by a linear gradient from 75% water/25% acetonitrile to 70% water/30% acetonitrile from 3-30 minutes. The prominent peak was collected at ˜14.7 minutes using this gradient and confirmed with LC-MS analysis.

### Cloacaenodin Stability

A 200 μL sample of purified ˜18 μM cloacaenodin in water was incubated at 37° C. for 72 hours. 30 μL of the sample was injected on LC-MS at 24-hour increments.

### NMR Data Collection

NMR studies were performed at the Princeton University Department of Chemistry NMR Facilities using a Bruker Avance III HD 800 MHz NMR spectrometer. Purified cloacaenodin was prepared at a concentration of 6.7 mg/mL (2.6 mM) in 95:5 H₂O:D₂O. The NMR spectra were acquired at 4° C. to minimize any cloacaenodin unthreading. A ¹H-¹H TOCSY spectrum at a mixing time of 80 ms was acquired, as well as 1H-1H NOESY spectra at 150 ms and 300 ms. Water suppression was used in the collection of all NMR data. To verify that the lasso peptide was not undergoing unthreading, 1D 1H NMR data were collected in between each 2D NMR acquisition and analyzed to see that the 1D spectra stayed consistent. As further confirmation that the lasso peptide had not undergone any conformational change during the NMR acquisition and that no degradation or contamination of the sample had occurred, an aliquot of the NMR sample was injected onto LC-MS following NMR data collection.

### Determination of Structure Through NMR Analysis

NMR spectra were processed and analyzed with the use of MNova (Mestrelab). Residues were manually assigned from an overlay of the 80 ms TOCSY and the 300 ms NOESY. After residue assignment, the 150 ms NOESY was used for through-space distance measurements, where cross-peaks were manually chosen and integrated. These peaks were inputted to CYANA 2.1 to be used as distance constraints. Further explicit distance constraints were inputted regarding the amino acids involved in the isopeptide bond (Gly 1 (G1) and Glu9 (E9)) and are listed in Table 14. These distances were calculated from the crystal structure of the similarly 9-member (9-residue) ringed lasso peptide rubrivinodin⁵³ (PDB 50QZ).

TABLE 14
Explicit distance constraints used for
cloacaenodin model building in CYANA.

| Residue | | | Residue | | | Distance |
|---|---|---|---|---|---|---|
| Number | Residue | Atom | Number | Residue | Atom | (Angstroms) |
| 1 | Gly | N | 9 | Glu | CD | 1.33 |
| 1 | Gly | N | 9 | Glu | OE1 | 2.26 |
| 1 | Gly | N | 9 | Glu | CG | 2.42 |
| 1 | Gly | H | 9 | Glu | CD | 2.06 |
| 1 | Gly | CA | 9 | Glu | CD | 2.47 |
| 1 | Gly | H | 1 | Gly | CA | 2.02 |
| 1 | Gly | CA | 9 | Glu | OE1 | 2.82 |
| 1 | Gly | H | 9 | Glu | OE1 | 3.15 |

The CYANA analysis program was used for seven cycles of initial structural calculations, with 100 initial structures, resulting in 20 final structures. These 20 structures were then energy minimized using Avogadro, with force field MMFF94 and the steepest descent algorithm used.

### Cloacaenodin Antimicrobial Activity

Cloacaenodin was evaluated against several common laboratory strains as well as commercially acquired strains using a spot-on-lawn assay.⁴⁵ Strains were streaked out onto LB agar plates and incubated overnight at their recommended temperatures. A single colony was then used to inoculate an overnight culture in 5 mL of LB broth. After shaking at 250 rpm at the recommended temperature for each strain, 50 μL of the overnight culture was used to inoculate 5 mL of LB broth (volume ratio 1:100). These cultures were grown for a few hours until they reached the exponential phase (OD₆₀₀˜0.4-0.6) before being added to 10 mL of soft M63 agar at a final cell density of 10⁷ CFUs/mL, or 10⁸ CFUs total in 10 mL. The M63 soft agar was composed of 2 g/L of (NH₄)₂SO₄ (EMD MilliporeSigma), 13.6 g/L of KH₂PO₄ (Fisher), 40 mg/L of each of the 20 common amino acids, 0.2% glucose (Sigma), 0.00005% w/v thiamine hydrochloride (Sigma), and 0.65% w/v bacteriological-grade agar (Apex Bioresearch). The inoculated agar was then poured on top of a 10 mL M63 hard agar plate (contains same components of M63 soft agar but is instead 1.5% w/v agar and does not contain amino acids) and allowed to cool. Upon solidification, 10 μL of purified cloacaenodin in water at two-fold dilutions were then spotted onto the agar and allowed to dry. The plates were incubated overnight at the strains' recommended temperatures and analyzed the next morning for activity. The MIC is defined as the last dilution where a spot was visible.

Twelve (12) clinical isolates were tested at the Broad Institute of MIT and Harvard, with a subset of strains from BioProject PRJNA292902, BioProject PRJNA271899, BioProject PRJNA201976, and BioProject 219285. The strains were tested following the same spot-on-lawn assay protocol described above, but with BD Bacto Agar used instead. All cloacaenodin-treated isolates were incubated at 37° C. overnight, and the assay was repeated at least three times (on biological replicates) for each of the twelve (12) strains. We defined a strain to be susceptible if it was reliably susceptible in at least three biological replicates.

For liquid inhibition assays, 5 mL LB broth was inoculated with 40-50 μL of an overnight culture of E. cloacae or E. amnigenus. Once the culture reached the exponential phase, the culture was diluted to an OD₆₀₀ of 0.0005 in a 96-well plate in M63 media (same components as M63 soft agar but lacks agar) with varying concentrations of cloacaenodin. The plate was shaken at 30° C. at 250 rpm for E. cloacae, and 37° C. at 250 rpm for E. amnigenus. The OD₆₀₀ was measured at 8-hour and 16-hour increments. The MIC is defined as the lowest concentration of cloacaenodin for which growth (as assessed by the OD₆₀₀) was inhibited.

### Microscopy

After a 96-well plate of E. cloacae grew for 16 hours at 30° C. with varying concentrations of cloacaenodin in M63 media, samples were imaged using a Zeiss Observer Z1 automated inverted microscope with a cage incubator kept at 37° C. The microscope was used with a 100× Zeiss chroma objective with oil immersion and 35 ms exposure transmitted light and controlled using SlideBook software. Images were viewed using the software ImageJ.

### Carboxypeptidase Digestion

Carboxypeptidase assays were done in 50 μL of 50 mM sodium acetate, pH 6.0 with 1 unit each of carboxypeptidase B and carboxypeptidase Y. Reactions were incubated overnight at 20° C. for 16 hours and then quenched with 1% formic acid. An aliquot of the reaction was then injected onto LC-MS to compare with an untreated control.

### Protease Digestion

Sequencing grade trypsin (Promega) was added to 50 μM peptide samples at a 1:100 trypsin: peptide weight ratio in a buffer of 50 mM ammonium bicarbonate. The reaction was allowed to proceed at room temperature for 30 minutes to 1 hour and then quenched by 1% formic acid. An aliquot was then injected onto LC-MS for analysis.

α-chymotrypsin from bovine pancreas (Sigma-Aldrich) was first resuspended in 1 mM HCl, 2 mM CaCl₂). The enzyme was added to 50 μM peptide samples at a final enzyme concentration of about 0.04 mg/mL in a buffer of 100 mM tris(hydroxymethyl)aminomethane (Tris), 10 mM CaCl₂, pH 8. The reactions were then allowed to incubate at 25° C. for about 1 hour before quenching with 1% formic acid, and an aliquot was injected onto LC-MS for analysis.

Elastase (Promega) was resuspended in 50 mM Tris pH 9.0. The enzyme was added to 50 μM peptide samples at a final enzyme concentration of about 0.04 mg/mL in a buffer of 50 mM Tris, pH 9. The reactions were then allowed to incubate at 25° C. for about 1 hour before quenching with 1% formic acid. An aliquot was then injected into LC-MS for analysis.

Thermolysin from Geobacillus stearothermophilus (Sigma-Aldrich) was first resuspended in 50 mM Tris, 0.5 mM CaCl₂). The enzyme was then added to 50 μM peptide samples at a final concentration of about 0.04 mg/mL in a buffer of 50 mM Tris, 0.5 mM CaCl₂), pH 8. The reactions were then allowed to incubate at 30° C. for about 1 hour before quenching with 1% formic acid. An aliquot was then injected onto LC-MS for analysis.

### Data Deposition

The structure of cloacaenodin with its atomic coordinates has been deposited to the Protein Data Bank under PDB code 8DYN and to the Biological Magnetic Resonance Data Bank under BMRB entry 31037: these deposits to the Protein Data Bank and the Biological Magnetic Resonance Data Bank are hereby incorporated by reference in their entirety.

### Pharmaceutical Compositions and Administration

A pharmaceutical composition can include a cloacaenodin-class lasso peptide and a pharmaceutically acceptable carrier or diluent.

A cloacaenodin-class lasso peptide according to the invention can be formulated as a pharmaceutical composition and administered to a patient or subject in need of treatment in a form adapted to the chosen route of administration, for example, intravenously, intraperitoneally, intramuscularly, subcutaneously, intradermally, by injection into tissue, orally, by nasal insufflation, by inhalation, topically, vaginally, urethrally, or rectally.

Thus, a cloacaenodin-class lasso peptide of the invention may be systemically administered, e.g., orally, in combination with a pharmaceutically acceptable vehicle such as an inert diluent or an assimilable edible carrier, or by inhalation or insufflation. It may be enclosed in hard or soft shell gelatin capsules, may be compressed into tablets, or may be incorporated directly with the food of the patient's diet. For oral therapeutic administration, a cloacaenodin-class lasso peptide may be combined with one or more excipients and used in the form of ingestible tablets, buccal tablets, troches, capsules, elixirs, suspensions, syrups, wafers, and the like. A cloacaenodin-class lasso peptide may be combined with a fme inert powdered carrier and inhaled by the subject or insufflated. Such compositions and preparations may contain at least 0.1% of a cloacaenodin-class lasso peptide. The percentage of the compositions and preparations may be varied and, for example, may be between about 2% to about 60% of the weight of a given unit dosage form. The amount of a cloacaenodin-class lasso peptide in such therapeutically useful compositions is such that an effective dosage level will be obtained.

The tablets, troches, pills, capsules, and the like may also contain the following: binders such as gum tragacanth, acacia, corn starch or gelatin: excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, a cloacaenodin-class lasso peptide may be incorporated into sustained-release preparations and devices. For example, a cloacaenodin-class lasso peptide may be incorporated into time release capsules, time release tablets, and time release pills.

A cloacaenodin-class lasso peptide may be administered intravenously or intraperitoneally by infusion or injection. Solutions of a cloacaenodin-class lasso peptide can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

Pharmaceutical dosage forms suitable for injection or infusion can include sterile aqueous solutions or dispersions or sterile powders including a cloacaenodin-class lasso peptide which are adapted for the extemporaneous preparation of sterile injectable or infusible solutions or dispersions, optionally encapsulated in liposomes. In all cases, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. It may be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by a cloacaenodin-class lasso peptide in the required amount in the appropriate solvent with various of the other ingredients enumerated above, as required, followed by filter sterilization. In the case of sterile powders for the preparation of sterile injectable solutions, the preferred methods of preparation are vacuum drying and freeze-drying techniques, which yield a powder of the active ingredient plus any additional desired ingredient present in the previously sterile-filtered solutions.

For topical administration, a cloacaenodin-class lasso peptide may be applied in pure form. However, it may be desirable to administer it to the skin as a composition or formulation, in combination with a dermatologically acceptable carrier, which may be a solid or a liquid.

Useful solid carriers include finely divided solids such as talc, clay, microcrystalline cellulose, silica, alumina and the like. Other solid carriers include nontoxic polymeric nanoparticles or microparticles. Useful liquid carriers include water, alcohols or glycols or water/alcohol/glycol blends, in which a cloacaenodin-class lasso peptide can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of a cloacaenodin-class lasso peptide can be determined by comparing its in vitro activity and its in vivo activity in animal models.

For example, the concentration of a cloacaenodin-class lasso peptide in a liquid composition, such as a lotion, can be from about 0.1-25% by weight, or from about 0.5-10% by weight. The concentration in a semi-solid or solid composition such as a gel or a powder can be about 0.1-5% by weight, or about 0.5-2.5% by weight.

The amount of a cloacaenodin-class lasso peptide required for use in treatment will vary with the route of administration, the nature of the condition being treated and the age and condition of the patient and will be ultimately at the discretion of the attendant physician or clinician.

Effective dosages and routes of administration of agents of the invention may be conventional. The exact amount (effective dose) of the agent will vary from subject to subject, depending on, for example, the species, age, weight and general or clinical condition of the subject, the severity or mechanism of any disorder being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either in cell culture assays or in suitable animal models. The animal model may also be used to determine the appropriate concentration ranges and routes of administration. Such information can then be used to determine useful doses and routes for administration in humans. A therapeutic dose can also be selected by analogy to dosages for comparable therapeutic agents.

The particular mode of administration and the dosage regimen will be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

A suitable dose may be in the range of from about 0.001 to about 100 mg/kg, e.g., from about 0.01 to about 100 mg/kg of body weight per day, such as above about 0.1 mg per kilogram, or in a range of from about 1 to about 10 mg per kilogram body weight of the recipient per day. For example, a suitable dose may be about 1 mg/kg, 10 mg/kg, or 50 mg/kg of body weight per day.

A cloacaenodin-class lasso peptide may be conveniently administered in unit dosage form: for example, containing 0.05 to 10000 mg, 0.5 to 10000 mg, 5 to 1000 mg, or about 100 mg of active ingredient per unit dosage form.

A cloacaenodin-class lasso peptide can be administered to achieve peak plasma concentrations of, for example, from about 0.1 to about 200 M, 0.2 to about 100 μM, 0.5 to about 75 μM, about 1 to 50 μM, about 2 to about 30 μM, or about 5 to about 25 μM. Exemplary desirable plasma concentrations include at least or no more than 0.1, 0.25, 0.5, 1, 5, 10, 25, 50, 75, 100 or 200 μM. For example, plasma levels may be from about 1 to 100 micromolar or from about 10 to about 25 micromolar. This may be achieved, for example, by the intravenous injection of a 0.05 to 5% solution of a cloacaenodin-class lasso peptide, optionally in saline, or orally administered as a bolus containing about 1-100 mg of the cloacaenodin-class lasso peptide. Desirable blood levels may be maintained by continuous infusion to provide about 0.00005-5 mg per kg body weight per hour, for example at least or no more than 0.00005, 0.0005, 0.005, 0.05, 0.5, or 5 mg/kg/hr. Alternatively, such levels can be obtained by intermittent infusions containing about 0.0002-20 mg per kg body weight, for example, at least or no more than 0.0002, 0.002, 0.02, 0.2, 2, 20, or 50 mg of the cloacaenodin-class lasso peptide per kg of body weight.

A cloacaenodin-class lasso peptide may conveniently be presented in a single dose or as divided doses administered at appropriate intervals, for example, as two, three, four or more sub-doses per day. The sub-dose itself may be further divided, e.g., into a number of discrete loosely spaced administrations: such as multiple inhalations from an insufflator.

### Aspects of the Invention

Aspect 1. A method for inhibiting growth of a microorganism,
providing a cloacaenodin-class lasso peptide that is purified; and
exposing the microorganism to the cloacaenodin-class lasso peptide,
so that the growth of the microorganism is inhibited,
wherein the cloacaenodin-class lasso peptide comprises a ring, a loop region, and a tail region,
wherein the cloacaenodin-class lasso peptide comprises a peptide sequence GHSVDRIPEYFGPPGLPGPVLFYS [SEQ ID NO. 1],
wherein 0, 1, or 2 of residues 2 through 8 of the peptide sequence are removed or replaced with another residue,
wherein 0 or 1 residue is inserted after one of residues 1 through 8,
wherein 0, 1, 2, 3, 4, or 5 of residues 12 through 24 of the peptide sequence are removed or replaced, and
wherein 0, 1, 2, 3, or 4 residues are inserted after at least one of residues 11 through 21.

Aspect 2. The method according to aspect 1, wherein the cloacaenodin-class lasso peptide is not cloacaenodin of the peptide sequence GHSVDRIPEYFGPPGLPGPVLFYS [SEQ ID NO. 1], not cloacaenodin-2 of the peptide sequence GHSVDRIPEYFGPPGLPSPVLFYS [SEQ ID NO. 105], and not cloacaenodin-3 of the peptide sequence GHSVDRIPEYFGPPGGPILFYS [SEQ ID NO. 106].

Aspect 3. The method according to any one of aspects 1 and 2, wherein the ring is of 9 residues.

Aspect 4. The method according to any one of aspects 1 and 2, wherein the ring is of a subsequence GHSVDRIPE [SEQ ID NO. 9].

Aspect 5. The method according to any one of aspects 1 and 2, wherein the ring is of 10 residues.

Aspect 6. The method according to any one of aspects 1 and 2, wherein the ring is of a subsequence GHSVADRIPE [SEQ ID NO. 7].

Aspect 7. The method according to any one of aspects 1 through 6, wherein the loop region is of 13 residues.

Aspect 8. The method according to any one of aspects 1 through 6, wherein the loop region is of a subsequence YFGPPGLPGPVLF [SEQ ID NO. 115].

Aspect 9. The method according to any one of aspects 1 through 6, wherein the loop region is of 12 residues or 11 residues.

Aspect 10. The method according to any one of aspects 1 through 9, wherein the tail region is of 2 residues.

Aspect 11. The method according to any one of aspects 1 through 9, wherein the tail region is of a subsequence YS.

Aspect 12. The method according to any one of aspects 1 through 11, wherein the cloacaenodin-class lasso peptide is threaded.

Aspect 13. The method according to any one of aspects 1 through 12, wherein residues 22 through 24 of the peptide sequence are not removed or replaced.

Aspect 14. The method according to any one of aspects 1 through 13, wherein at most 2 of residues 12 through 21 of the peptide sequence are removed or replaced.

Aspect 15. The method according to any one of aspects 1 through 14, wherein residue 8 of the peptide sequence is proline (P).

Aspect 16. The method according to any one of aspects 1 through 14, wherein residue 8 of the peptide sequence is replaced by alanine (A).

Aspect 17. The method according to any one of aspects 1 through 16, wherein residue 4 of the peptide sequence is replaced by proline (P).

Aspect 18. The method according to any one of aspects 1 through 17, wherein residue 22 of the peptide sequence is replaced by tryptophan (W).

Aspect 19. The method according to any one of aspects 1 through 18, wherein residue 23 of the peptide sequence is replaced by tryptophan (W).

Aspect 20. The method according to any one of aspects 1 through 19, wherein residue 24 of the peptide sequence is replaced by alanine (A).

Aspect 21. The method according to any one of aspects 1 through 19, wherein residue 24 of the peptide sequence is replaced by tyrosine (Y).

Aspect 22. The method according to any one of aspects 1 through 19, wherein residue 24 of the peptide sequence is replaced by threonine (T).

Aspect 23. The method according to any one of aspects 1 through 19, wherein residue 24 of the peptide sequence is replaced by cysteine (C).

Aspect 24. The method according to any one of aspects 1 through 23, wherein residue 10 of the peptide sequence is replaced by alanine (A)

Aspect 25. The method according to any one of aspects 1 through 24, wherein alanine (A) is inserted after residue 4 in the peptide sequence.

Aspect 26. The method according to any one of aspects 1 through 25, wherein residue 18 of the peptide sequence is serine (S).

Aspect 27. The method according to any one of aspects 1 through 26, wherein residue 20 of the peptide sequence is isoleucine (I).

Aspect 28. The method according to any one of aspects 1 through 27, wherein residues 16 and 17 of the peptide sequence are removed.

Aspect 29. The method according to aspect 1, wherein the cloacaenodin-class lasso peptide is cloacaenodin of a peptide sequence GHSVDRIPEYFGPPGLPGPVLFYS [SEQ ID NO. 1]

Aspect 30. The method according to aspect 1, wherein the cloacaenodin-class lasso peptide is cloacaenodin-2 of a peptide sequence GHSVDRIPEYFGPPGLPSPVLFYS [SEQ ID NO. 105].

Aspect 31. The method according to aspect 1, wherein the cloacaenodin-class lasso peptide is cloacaenodin-3 of a peptide sequence GHSVDRIPEYFGPPGGPILFYS [SEQ ID NO. 106].

Aspect 32. The method according to aspect 1, wherein the cloacaenodin-class lasso peptide is of a peptide sequence GHSVDRIPEYFGPPGLPGPVLWYS [SEQ ID NO. 111].

Aspect 33. The method according to aspect 1, wherein the cloacaenodin-class lasso peptide is of a peptide sequence GHSVDRIPEYFGPPGLPGPVLFYA [SEQ ID NO. 113].

Aspect 34. The method according to claim 1, wherein the cloacaenodin-class lasso peptide is of a peptide sequence GHSVDRIPEYFGPPGLPGPVLFYY [SEQ ID NO. 114].

Aspect 35. The method according to aspect 1, wherein the cloacaenodin-class lasso peptide is of a peptide sequence selected from the group consisting of

[SEQ ID NO. 67]
GHSVADRIPEYFGPPGLPGPVLFYS,
[SEQ ID NO. 109]
GHSVDRIAEYFGPPGLPGPVLFYS,
[SEQ ID NO. 110]
GHSPDRIPEYFGPPGLPGPVLFYS,
[SEQ ID NO. 112]
GHSVDRIPEYFGPPGLPGPVLFWS,
[SEQ ID NO. 116]
GHSVDRIPEYFGPPGLPGPVLFYT,
[SEQ ID NO. 117]
GHSVDRIPEYFGPPGLPGPVLFYC,
and
[SEQ ID NO. 118]
GHSVDRIPEAFGPPGLPGPVLFYS.

Aspect 36. The method according to any one of aspects 1 through 28, wherein the peptide sequence is at least 85% homologous to GHSVDRIPEYFGPPGLPGPVLFYS [SEQ ID NO. 1].

Aspect 37. The method according to any one of aspects 1 through 28, wherein the peptide sequence is at least 95% homologous to GHSVDRIPEYFGPPGLPGPVLFYS [SEQ ID NO. 1].

Aspect 38. The method according to any one of aspects 1, 2, 4, and 7 through 37,
wherein the ring is of 9 residues and
wherein the cloacaenodin-class lasso peptide comprises a threaded structure.

Aspect 39. The method according to any one of aspects 1 through 38, wherein the microorganism is a gram-negative gammaproteobacterium.

Aspect 40. The method according to any one of aspects 1 through 38, wherein the microorganism is of order Enterobacterales.

Aspect 41. The method according to any one of aspects 1 through 38, wherein the microorganism is of family Enterobacteriaceae.

Aspect 42. The method according to any one of aspects 1 through 38, wherein the microorganism is a species of Enterobacter.

Aspect 43. The method according to any one of aspects 1 through 38, wherein the microorganism is Enterobacter amnigenus, Enterobacter asburiae, Enterobacter mori, or Enterobacter nimipressuralis.

Aspect 44. The method according to any one of aspects 1 through 38, wherein the microorganism is Enterobacter cloacae.

Aspect 45. The method according to any one of aspects 1 through 38, wherein the microorganism is Enterobacter hormaechei, Enterobacter kobei, or Enterobacter ludwigii.

Aspect 46. The method according to any one of aspects 1 through 38, wherein the microorganism is Enterobacter xiangfangensis.

Aspect 47. The method according to any one of aspects 1 through 38, wherein the microorganism is a species of Kluyvera.

Aspect 48. The method according to any one of aspects 1 through 38, wherein the microorganism is Kluyvera ascorbata.

Aspect 49. The method according to any one of aspects 1 through 48, wherein the microorganism is resistant to an antibiotic, resistant to a broad spectrum antibiotic, resistant to an antibiotic of last resort, or resistant to a beta-lactam antibiotic.

Aspect 50. The method according to any one of aspects 1 through 48, wherein the microorganism is resistant to a carbapenem.

Aspect 51. The method according to any one of aspects 1 through 48,
wherein providing the cloacaenodin-class lasso peptide comprises providing a pharmaceutical composition comprising the cloacaenodin-class lasso peptide and
wherein the pharmaceutical composition further comprises a pharmaceutically acceptable carrier or diluent.

Aspect 52. The method according to aspect 51, wherein the lasso peptide is present in the pharmaceutical composition at a concentration of less than 10 μM.

Aspect 53. The method according to any one of aspects 51 and 52, wherein the pharmaceutical composition is of a dosage form selected from the group consisting of an injectable liquid, a capsule, a tablet, a pill, a suppository, a powder, a time-release capsule, a time-release table, a time release pill, a time-release suppository, a cream, an ointment, a gel, and an impregnated wound dressing.

Aspect 54. The method according to any one of aspects 1 through 53, wherein the microorganism is exposed to the cloacaenodin-class lasso peptide in vitro.

Aspect 55. The method according to any one of aspects 1 through 54, wherein the cloacaenodin-class lasso peptide is of a minimal inhibitory concentration (MIC) against the microorganism of 8 μM or less.

Aspect 56. The method according to any one of aspects 1 through 54, wherein the cloacaenodin-class lasso peptide is of a minimal inhibitory concentration (MIC) against the microorganism of 4 μM or less.

Aspect 57. The method according to any one of aspects 1 through 54, wherein the cloacaenodin-class lasso peptide is of a minimal inhibitory concentration (MIC) against the microorganism of 2 μM or less.

Aspect 58. The method according to any one of aspects 1 through 53, wherein the microorganism is exposed to the cloacaenodin-class lasso peptide within or on a patient.

Aspect 59. A method of treating a patient infected with the microorganism, comprising administering the cloacaenodin-class lasso peptide to the patient according to the method of any one of aspects 1 through 53, thereby treating the patient.

Aspect 60. A method of treating a patient to prevent infection with the microorganism, comprising administering the cloacaenodin-class lasso peptide to the patient according to the method of any one of aspects 1 through 53, thereby preventing infection of the patient with the microorganism.

Aspect 61. The method of treating a patient according to any one of aspects 59 and 60, wherein the cloacaenodin-class lasso peptide is administered to the patient intravenously, intraperitoneally, intramuscularly, subcutaneously, intradermally, by injection into tissue, orally, by nasal insufflation, by inhalation, topically, vaginally, urethrally, or rectally.

Aspect 62. A purified cloacaenodin-class lasso peptide, comprising
a peptide sequence GHSVDRIPEYFGPPGLPGPVLFYS [SEQ ID NO. 1],
wherein the peptide sequence comprises a ring, a loop region, and a tail region,
wherein the ring is bonded to the loop region,
wherein the loop region is bonded to the tail region,
wherein 0, 1, or 2 of residues 2 through 8 of the peptide sequence are removed or replaced with another residue,
wherein 0 or 1 residue is inserted after one of residues 1 through 8,
wherein 0, 1, 2, 3, 4, or 5 of residues 12 through 24 of the peptide sequence are removed or replaced, and
wherein 0, 1, 2, 3, or 4 residues are inserted after residues 11 through 21.

Aspect 63. The purified cloacaenodin-class lasso peptide according to aspect 62, wherein the purified cloacaenodin-class lasso peptide is not cloacaenodin of the peptide sequence GHSVDRIPEYFGPPGLPGPVLFYS [SEQ ID NO. 1], not cloacaenodin-2 of the peptide sequence GHSVDRIPEYFGPPGLPSPVLFYS [SEQ ID NO. 105], and not cloacaenodin-3 of the peptide sequence GHSVDRIPEYFGPPGGPILFYS [SEQ ID NO. 106].

Aspect 64. The purified cloacaenodin-class lasso peptide according to any one of aspects 62 and 63, wherein the ring is of 9 residues.

Aspect 65. The purified cloacaenodin-class lasso peptide according to any one of aspects 62 through 64, wherein the ring comprises a subsequence GHSVDRIPE [SEQ ID NO. 9].

Aspect 66. The purified cloacaenodin-class lasso peptide according to any one of aspects 62 and 63, wherein the ring is of 10 residues.

Aspect 67. The purified cloacaenodin-class lasso peptide according to any one of aspects 62, 63, and 66, wherein the ring comprises a subsequence GHSVADRIPE [SEQ ID NO. 7].

Aspect 68. The purified cloacaenodin-class lasso peptide according to any one of aspects 62 through 67, wherein the loop region is of 13 residues.

Aspect 69. The purified cloacaenodin-class lasso peptide according to any one of aspects 62-68, wherein the loop region comprises a subsequence YFGPPGLPGPVLF [SEQ ID NO. 115].

Aspect 70. The purified cloacaenodin-class lasso peptide according to any one of aspects 62 through 67, wherein the loop region is of 12 residues or 11 residues.

Aspect 71. The purified cloacaenodin-class lasso peptide according to any one of aspects 62 through 70, wherein the tail region is of 2 residues.

Aspect 72. The purified cloacaenodin-class lasso peptide according to any one of aspects 62 through 71, wherein the tail region comprises a subsequence YS.

Aspect 73. The purified cloacaenodin-class lasso peptide according to any one of aspects 62 through 72,
wherein the tail region is threaded through the ring and
wherein the tail region and the loop region are on opposite sides of the ring.

Aspect 74. The purified cloacaenodin-class lasso peptide according to any one of aspects 62 through 73, wherein the tail region of a subsequence YS is threaded through the ring of a subsequence GHSVDRIPE [SEQ ID NO. 9].

Aspect 75. The purified cloacaenodin-class lasso peptide according to any one of aspects 62 through 74, wherein residues 22 through 24 of the peptide sequence are not removed or replaced.

Aspect 76. The purified cloacaenodin-class lasso peptide according to any one of aspects 62 through 75, wherein at most 2 of residues 12 through 21 of the peptide sequence are removed or replaced.

Aspect 77. The purified cloacaenodin-class lasso peptide according to any one of aspects 62 through 76, wherein residue 8 of the peptide sequence is proline (P).

Aspect 78. The purified cloacaenodin-class lasso peptide according to any one of aspects 62 through 76, wherein residue 8 of the peptide sequence is alanine (A).

Aspect 79. The purified cloacaenodin-class lasso peptide according to any one of aspects 62 through 78, wherein residue 4 of the peptide sequence is proline (P).

Aspect 80. The purified cloacaenodin-class lasso peptide according to any one of aspects 62 through 79, wherein residue 22 of the peptide sequence is tryptophan (W).

Aspect 81. The purified cloacaenodin-class lasso peptide according to any one of aspects 62 through 80, wherein residue 23 of the peptide sequence is tryptophan (W).

Aspect 82. The purified cloacaenodin-class lasso peptide according to any one of aspects 62 through 81, wherein residue 24 of the peptide sequence is alanine (A).

Aspect 83. The purified cloacaenodin-class lasso peptide according to any one of aspects 62 through 81, wherein residue 24 of the peptide sequence is tyrosine (Y).

Aspect 84. The purified cloacaenodin-class lasso peptide according to any one of aspects 62 through 81, wherein residue 24 of the peptide sequence is threonine (T).

Aspect 85. The purified cloacaenodin-class lasso peptide according to any one of aspects 62 through 81, wherein residue 24 of the peptide sequence is cysteine (C).

Aspect 86. The purified cloacaenodin-class lasso peptide according to any one of aspects 62 through 85, wherein residue 10 of the peptide sequence is alanine (A).

Aspect 87. The purified cloacaenodin-class lasso peptide according to any one of aspects 62 through 86, wherein alanine (A) is inserted after residue 4 in the peptide sequence.

Aspect 88. The purified cloacaenodin-class lasso peptide according to any one of aspects 62 through 87, wherein residue 18 of the peptide sequence is serine (S).

Aspect 89. The purified cloacaenodin-class lasso peptide according to any one of aspects 62 through 88, wherein residue 20 of the peptide sequence is isoleucine (I).

Aspect 90. The purified cloacaenodin-class lasso peptide according to any one of aspects 62 through 89, wherein one or both of residues 16 and 17 of the peptide sequence are removed.

Aspect 91. The purified cloacaenodin-class lasso peptide according to aspect 62, wherein the peptide sequence is GHSVDRIPEYFGPPGLPGPVLFYS [SEQ ID NO. 1]

Aspect 92. The purified cloacaenodin-class lasso peptide according to aspect 62, wherein the peptide sequence is GHSVDRIPEYFGPPGLPSPVLFYS [SEQ ID NO. 105].

Aspect 93. The purified cloacaenodin-class lasso peptide according to aspect 62, wherein the peptide sequence is GHSVDRIPEYFGPPGGPILFYS [SEQ ID NO. 106].

Aspect 94. The purified cloacaenodin-class lasso peptide according to aspect 62, wherein the peptide sequence is GHSVDRIPEYFGPPGLPGPVLWYS [SEQ ID NO. 111].

Aspect 95. The purified cloacaenodin-class lasso peptide according to aspect 62, wherein the peptide sequence is GHSVDRIPEYFGPPGLPGPVLFYA [SEQ ID NO. 113].

Aspect 96. The purified cloacaenodin-class lasso peptide according to aspect 62, wherein the peptide sequence is GHSVDRIPEYFGPPGLPGPVLFYY [SEQ ID NO. 114].

Aspect 97. The purified cloacaenodin-class lasso peptide according to aspect 62, wherein the peptide sequence is selected from the group consisting of

[SEQ ID NO. 67]
GHSVADRIPEYFGPPGLPGPVLFYS,
[SEQ ID NO. 109]
GHSVDRIAEYFGPPGLPGPVLFYS,
[SEQ ID NO. 110]
GHSPDRIPEYFGPPGLPGPVLFYS,
[SEQ ID NO. 112]
GHSVDRIPEYFGPPGLPGPVLFWS,
[SEQ ID NO. 116]
GHSVDRIPEYFGPPGLPGPVLFYT,
[SEQ ID NO. 117]
GHSVDRIPEYFGPPGLPGPVLFYC,
and
[SEQ ID NO. 118]
GHSVDRIPEAFGPPGLPGPVLFYS.

Aspect 98. The purified cloacaenodin-class lasso peptide according to any one of aspects 62 through 90, wherein the peptide sequence is at least 85% homologous to

[SEQ ID NO. 1]
GHSVDRIPEYFGPPGLPGPVLFYS.

Aspect 99. The purified cloacaenodin-class lasso peptide according to any one of aspects 62 through 90, wherein the peptide sequence is at least 95% homologous to

[SEQ ID NO. 1]
GHSVDRIPEYFGPPGLPGPVLFYS.

Aspect 100. A pharmaceutical composition comprising the purified cloacaenodin-class lasso peptide according to any one of aspects 62 through 99 and a pharmaceutically acceptable carrier or diluent.

Aspect 101. The pharmaceutical composition according to aspect 100 of a dosage form selected from the group consisting of an injectable liquid, a capsule, a tablet, a pill, a suppository, a powder, a time-release capsule, a time-release table, a time release pill, a time-release suppository, a cream, an ointment, a gel, or an impregnated wound dressing.

Aspect 102. The purified cloacaenodin-class lasso peptide according to any one of aspects 62 through 99 for use as a medicament.

Aspect 103. The purified cloacaenodin-class lasso peptide according to any one of aspects 62 through 99 for use in treatment of an infection with a microorganism.

Aspect 104. The purified cloacaenodin-class lasso peptide according to any one of aspects 62 through 99 for use in prevention of an infection with a microorganism.

Aspect 105. The use of the purified cloacaenodin-class lasso peptide according to any one of aspects 62 through 99 in the manufacture of a medicament for treatment of an infection with a microorganism.

Aspect 106. The use of the purified cloacaenodin-class lasso peptide according to any one of aspects 62 through 99 in the manufacture of a medicament for prevention of an infection with a microorganism.

Aspect 107. The purified cloacaenodin-class lasso peptide according to any one of aspects 103 and 104 or the use of the purified cloacaenodin-class lasso peptide according to any one of aspects 105 and 106, wherein the microorganism is a gammaproteobacterium.

Aspect 108. The purified cloacaenodin-class lasso peptide according to any one of aspects 103 and 104 or the use of the purified cloacaenodin-class lasso peptide according to any one of aspects 105 and 106, wherein the microorganism is of order Enterobacterales.

Aspect 109. The purified cloacaenodin-class lasso peptide according to any one of aspects 103 and 104 or the use of the purified cloacaenodin-class lasso peptide according to any one of aspects 105 and 106, wherein the microorganism is of family Enterobacteriaceae.

Aspect 110. The purified cloacaenodin-class lasso peptide according to any one of aspects 103 and 104 or the use of the purified cloacaenodin-class lasso peptide according to any one of aspects 105 and 106, wherein the microorganism is of genus Enterobacter.

Aspect 111. The purified cloacaenodin-class lasso peptide according to any one of aspects 103 and 104 or the use of the purified cloacaenodin-class lasso peptide according to any one of aspects 105 and 106, wherein the microorganism is Enterobacter amnigenus, Enterobacter asburiae, Enterobacter mori, or Enterobacter nimipressuralis.

Aspect 112. The purified cloacaenodin-class lasso peptide according to any one of aspects 103 and 104 or the use of the purified cloacaenodin-class lasso peptide according to any one of aspects 105 and 106, wherein the microorganism is Enterobacter cloacae.

Aspect 113. The purified cloacaenodin-class lasso peptide according to any one of aspects 103 and 104 or the use of the purified cloacaenodin-class lasso peptide according to any one of aspects 105 and 106, wherein the microorganism is Enterobacter hormaechei, Enterobacter kobei, or Enterobacter ludwigii.

Aspect 114. The purified cloacaenodin-class lasso peptide according to any one of aspects 103 and 104 or the use of the purified cloacaenodin-class lasso peptide according to any one of aspects 105 and 106, wherein the microorganism is Enterobacter xiangfangensis.

Aspect 115. The purified cloacaenodin-class lasso peptide according to any one of aspects 103 and 104 or the use of the purified cloacaenodin-class lasso peptide according to any one of aspects 105 and 106, wherein the microorganism is of genus Kluyvera.

Aspect 116. The purified cloacaenodin-class lasso peptide according to any one of aspects 103 and 104 or the use of the purified cloacaenodin-class lasso peptide according to any one of aspects 105 and 106, wherein the microorganism is Kluyvera ascorbata.

Aspect 117. A method of producing a cloacaenodin-class lasso peptide, comprising
refactoring the precursor, protease, cyclase, and exporter genes for cloacaenodin into a plasmid,
transforming the plasmid into cells,
growing the cells,
inducing expression of the cloacaenodin-class lasso peptide by the cells,
separating the cells and a supernatant, and
obtaining purified cloacaenodin-class lasso peptide from the supernatant.

Aspect 118. The method of producing a cloacaenodin-class lasso peptide according to aspect 117, further comprising freezing the purified cloacaenodin-class lasso peptide at −10° C. or less within 30 minutes of obtaining the purified cloacaenodin-class lasso peptide from the supernatant.

Aspect 119. The method of producing a cloacaenodin-class lasso peptide according to claim 118, wherein the purified cloacaenodin-class lasso peptide is frozen at −20° C. or less.

Aspect 120. The method of producing a cloacaenodin-class lasso peptide according to claim 118, wherein the purified cloacaenodin-class lasso peptide is frozen at −80° C. or less.

Aspect 121. The method of producing a cloacaenodin-class lasso peptide according to any one of aspects 117 through 120, wherein the purified cloacaenodin-class lasso peptide is cloacaenodin of a peptide sequence GHSVDRIPEYFGPPGLPGPVLFYS [SEQ ID NO. 1].

Aspect 122. The method of producing a cloacaenodin-class lasso peptide according to any one of aspects 117 through 120, further comprising
using site-directed mutagenesis to modify the plasmid,
wherein the purified cloacaenodin-class lasso peptide comprises a peptide sequence

[SEQ ID NO. 1]
GHSVDRIPEYFGPPGLPGPVLFYS,

wherein 0, 1, or 2 of residues 2 through 8 of the peptide sequence are removed or replaced with another residue,
wherein 0 or 1 residue is inserted after one of residues 1 through 8,
wherein 0, 1, 2, 3, 4, or 5 of residues 12 through 24 of the peptide sequence are removed or replaced, and
wherein 0, 1, 2, 3, or 4 residues are inserted after at least one of residues 11 through 21.

Aspect 123. The method of producing a cloacaenodin-class lasso peptide according to aspect 122, wherein the purified cloacaenodin-class lasso peptide is cloacaenodin-2 of a peptide sequence GHSVDRIPEYFGPPGLPSPVLFYS [SEQ ID NO. 105] or cloacaenodin-3 of a peptide sequence GHSVDRIPEYFGPPGGPILFYS [SEQ ID NO. 106].

Aspect 124. The method of producing a cloacaenodin-class lasso peptide according to aspect 122, wherein the purified cloacaenodin-class lasso peptide is of a peptide sequence selected from the group consisting of

[SEQ ID NO. 67]
GHSVADRIPEYFGPPGLPGPVLFYS,
[SEQ ID NO. 109]
GHSVDRIAEYFGPPGLPGPVLFYS,
[SEQ ID NO. 110]
GHSPDRIPEYFGPPGLPGPVLFYS,
[SEQ ID NO. 111]
GHSVDRIPEYFGPPGLPGPVLWYS,
[SEQ ID NO. 112]
GHSVDRIPEYFGPPGLPGPVLFWS,
[SEQ ID NO. 113]
GHSVDRIPEYFGPPGLPGPVLFYA,
[SEQ ID NO. 114]
GHSVDRIPEYFGPPGLPGPVLFYY,
[SEQ ID NO. 116]
GHSVDRIPEYFGPPGLPGPVLFYT,
[SEQ ID NO. 117]
GHSVDRIPEYFGPPGLPGPVLFYC,
and
[SEQ ID NO. 118]
GHSVDRIPEAFGPPGLPGPVLFYS.

### DISCUSSION

An embodiment of the invention is the antimicrobial lasso peptide cloacaenodin. Cloacaenodin exhibits potent antimicrobial activity against multiple members (species and strains) of the Enterobacter genus, including those implicated in nosocomial infections. Although other lasso peptides with antimicrobial activity against gram-negative bacteria, such as klebsidin, capistruin, and citrocin, have only modest potency, the minimal inhibitory concentration (MIC) of cloacaenodin is in the high nanomolar to single micromolar range for bacterial strains tested. This potency and the need to develop new antimicrobial interventions, for example, against the ESKAPE pathogen Enterobacter, makes cloacaenodin a promising antibiotic. Biosynthetic gene clusters (BGCs) related to cloacaenodin are present in five (5) different species of Enterobacter as well as other enterobacteria (FIG. 5). Several of the BGCs appear in genomic contexts consistent with being plasmid-borne. Without being bound by theory, the mobility of the cloacaenodin BGC may be due to horizontal gene transfer between these strains, which are part of the gut microbiota. Without being bound by theory, this horizontal gene transfer may occur because cloacaenodin confers a competitive advantage on the producing cells.

Cloacaenodin differs in structure from other antimicrobial lasso peptides that may target gram-negative bacteria. For example, cloacaenodin has a 9-membered (9-residue) ring and a C-terminal serine (Ser, S), in contrast to other lasso peptides, which have 8-membered (8-residue) rings and a C-terminal glycine (Gly, G) (FIG. 1). Cloacaenodin is unusually rich in proline (Pro, P) residues in having five (5) prolines, four (4) of which are in the 13 aa (amino acid residue) loop region. As discussed above, the threaded structure of cloacaenodin protects it from proteolysis (FIGS. 4, 8A-8B, and 17A-20D). Without being bound by theory, the large number (the high prevalence) of prolines in the cloacaenodin structure may also contribute to this proteolytic resistance. Proline cis/trans isomerization is known in lasso peptides⁷⁵, and so, in principle, cloacaenodin may exist as an ensemble of up to 32 (i.e., 2⁵) different conformers. Without being bound by theory, in addition to the likely role of proline in engendering cloacaenodin with proteolytic resistance, this conformational flexibility may also be related to cloacaenodin's antimicrobial activity.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### REFERENCES

(1) Pendleton, J. N.; Gorman, S. P.; Gilmore, B. F. Clinical elevance of the ESKAPE Pathogens. Expert Rev Anti-Infe 2013, 11 (3), 297-308. https://doi.org/10.1586/eri.13.12.
(2) Davin-Regli, A.; Pagès, J.-M. Enterobacter aerogenes and Enterobacter cloacae; Versatile Bacterial Pathogens Confronting Antibiotic Treatment. Front Microbiol 2015, 6, 392. https://doi.org/0.3389/fmicb.2015.00392.
(3) Annavajhala, M. K.; Gomez-Simmonds, A.; Uhlemann, A.-C. Multidrug-Resistant Enterobacter cloacae Complex Emerging as a Global, Diversifying Threat. Front Microbiol 2019, 10, 44. https://doi.org/10.3389/fmicb.2019.00044.
(4) Mezzatesta, M. L.; Gona, F.; Stefani. S. Enterobacter cloacae Complex: Clinical Impact and Emerging Antibiotic Resistance. Future Microbiol 2012, 7 (7), 887-902. https://doi.org/10.2217/fmb.12.61.
(5) Davin-Regli, A.; Lavigne, J.-P.; Pagès, J.-M. Enterobacter Spp.; Update on Taxonomy, Clinical Aspects, and Emerging Antimicrobial Resistance. Clin Microbiol Rev 2019, 32 (4). https://doi.org/10.1128/cmr.00002-19.
(6) Bialvaei, A. Z.; Kafil, H. S. Colistin, Mechanisms and Prevalence of Resistance. Curr Med Res Opin 2015, 31 (4), 707-721. https://doi.org/10.1185/03007995.2015.1018989.
(7) Nation, R. L., Li, J. Colistin in the 21st Century. Curr Opin Infect Dis 2009, 22 (6), 535-543. https://doi.org/10.1097/qco.0b013e328332e672.
(8) Zong, Z.; Feng, Y.; McNally, A. Carbapenem and Colistin Resistance in Enterobacter: Determinants and Clones. Trends Microbiol 2021, 29 (6), 473-476. https://doi.org/10.1016/j.tim.2020.12.009.
(9) Norgan, A. P.; Freese. J. M.; Tuin. P. M.; Cunningham, S. A.; Jeraldo, P. R., Patel, R. Carbapenem- and Colistin-Resistant Enterobacter cloacae from Delta, Colorado, in 2015. Antimicrob Agents Ch 2016, 60 (5), 3141-3144. https://doi.org/10.1128/aac.03055-15.
(10) Zeng, K.-J.; Doi, Y., Patil, S.; Huang. X.; Tian, G.-B. Emergence of the Plasmid-Mediated mcr-1 Gene in Colistin-Resistant Enterobacter aerogenes and Enterobacter cloacae. Antimicrob Agents Ch 2016, 60 (6), 3862-3863. https://doi.org/10.1128/aac.00345-16.
(11) Kang, K. N.; Klein, D. R.; Kazi, M. I.; Guérin, F.; Cattoir, V.; Brodbelt, J. S.; Boll, J. M. Colistin Heteroresistance in Enterobacter cloacae Is Regulated by PhoPQ-Dependent 4-Amino-4-Deoxy-1-Arabinose Addition to Lipid A. Mol Microbiol 2019, 111 (6), 1604-1616. https://doi.org/10.1111/mmi.14240.
(12) Liao, W.; Cui, Y.; Quan, J.; hao, D.; Han, X.; Shi, Q.; Wang, Q.; Jiang, Y.; Du, X.; Li, X.; Yu, Y. High Prevalence of Colistin Resistance and mcr-9/10 Genes in Enterobacter Spp. in a Tertiary Hospital over a Decade. Int J Antimicrob Ag 2022, 59 (5), 106573. https://doi.org/10.1016/j.ijantimicag.2022.106573.
(13) Salomón. R. A.; Farias. R. N. Microcin 25, a Novel Antimicrobial Peptide Produced by Escherichia coli. J Bacteriol 1992, 174 (22), 7428-7435. https://doi.org/0.1128/jb.174.22.7428-7435.1992.
(14) Knappe, T. A.; Linne, U.; Zirah, S.; Rebuffat, S.; Xie. X.; Marahiel, M. A. Isolation and Structural Characterization of Capistruin, a Lasso Peptide Predicted from the Genome Sequence of Burkholderia thailandensis E264. J Am Chem Soc 2008, 130 (34), 11446-11454. https://doi.org/10.1021/ja802966g.
(15) Iwatsuki, M.; Uchida, R.; Takakusagi; Matsumoto, A.; Jiang. C.-L.; Takahashi, Y.; Arai, M.; Kobayashi, S.; Matsumoto, M.; Inokoshi, J.; Tomoda, H.; Omura, S. Lariatins, Novel Anti-Mycobacterial Peptides with a Lasso Structure, Produced by Rhodococcus jostii K0-B0171. J Antibiot 2007, 60 (6), 357-363. https://doi.org/0.1038/ja.2007.48.
(16) Cheung-Lee, W. L.; Parry, M. E.; Zong, C.; Cartagena, A. J.; Darst. S. A.; Connell. N. D.; Russo, R.; Link, A. J. Discovery of Ubonodin, an Antimicrobial Lasso Peptide Active against Members of 1 the Burkholderia cepacia Complex. Chembiochem 2020, 21 (9), 1335-1340. https://doi.org/10.1002/cbic.201900707.
(17) Cheung-Lee, W. L.; Parry, M. E.; Cartagena, A. J.; Darst, S. A.; Link, A. J. Discovery and Structure of the Antimicrobial Lasso Peptide Citrocin. J Biol Chem 2019, 294 (17), 6822-6830. https://doi.org/10.1074/jbc.ra118.006494.
(18) Tsunakawa, M.; Hu, S-L.; Hoshino, Y.; Detlefson, D. J.; Hill, S. E.; Furumai, T.; White. R. J.; Nishio, M.; Kawano, K.; Yamamoto, S.; Fukagawa, Y, Oki, T., Siamycins I and II, New Anti-HIV Peptides: I. Fermentation, Isolation, Biological Activity and Initial Characterization. J Antibiot 1995, 48 (5), 433-434. https://doi.org/10.7164/antibiotics.48.433.
(19) Metelev, M.; Arseniev, A.; Bushin, L. B.; Kuznedelov, K.; Artamonova, T. O.; Kondratenko, R.; Khodorkovskii, M.; Seyedsayamdost, M. R.; Severinov, K. Acinetodin and Klebsidin, RNA Polymerase Targeting Lasso Peptides Produced by Human Isolates of Acinetobacter gyllenbergii and Klebsiella pneumoniae. ACS Chem Biol 2017, 12 (3), 814-824. https://doi.org/10.1021/acschembio.6b01154.
(20) Metelev, M.; Tietz. J. I.; Melby, J. O.; Blair, P. M.; Zhu, L.; Livnat, I.; Severinov, K.; Mitchell, D. A. Structure, Bioactivity, and Resistance Mechanism of Streptomonomicin, an Unusual Lasso Peptide from an Understudied Halophilic Actinomycete. Chem Biol 2015, 22 (2), 241-250. https://doi.org/10.1016/j.chembiol.2014.11.017.
(20) Gavrish, E.; Sit, C. S.; Cao, S.; Kandror, O.; Spoering, A.; Peoples. A.; Ling, L.; Fetterman, A.; Hughes, D.; Bissell, A.; Torrey, H.; Akopian, T.; Mueller, A.; Epstein, S.; Goldberg, A.; Clardy, J.; Lewis, K. Lassomycin, a Ribosomally Synthesized Cyclic Peptide, Kills Mycobacterium tuberculosis by Targeting the ATP-Dependent Protease ClpC1P1P2. Chem Biol 2014, 21 (4), 509-518. https://doi.org/10.1016/j.chembiol.2014.01.014.
(22) Stariha, L. M.; McCafferty, D. G. Discovery of the Class I Antimicrobial Lasso Peptide Arcumycin. Chembiochem 2021, 22 (16), 2632-2640. https://doi.org/10.1002/cbic.202100132.
(23) Li, Y.; Ducasse, R.; Zirah, S.; Blond, A.; Goulard, C.; Lescop, E.; Giraud, C.; Hartke, A.; Guittet, E.; Pernodet, J.-L.; Rebuffat, S. Characterization of Sviceucin from Streptomyces Provides Insight into Enzyme Exchangeability and Disulfide Bond Formation in Lasso Peptides. ACS Chem Biol 2015, 10 (11), 2641-2649. https://doi.org/10.1021/acschembio.5b00584.
(24) Shao, M.; Ma, J.; Li, Q.; Ju, J. Identification of the Anti-Infective Aborycin Biosynthetic Gene Cluster from Deep-Sea-Derived Streptomyces sp. SCSIO ZS0098 Enables Production in a Heterologous Host. Mar Drugs 2019, 17 (2), 127. https://doi.org/10.3390/md17020127.
(25) Montalbán-López, M.; Scott, T. A.; Ramesh, S.; Rahman, I. R.; Heel, A. J. van; Viel, J. H.; Bandarian, V.; Dittmann, E.; Genilloud, O.; Goto, Y.; Burgos, M. J. G.; Hill, C.; Kim, S.; Koehnke, J.; Latham, J. A.; Link, A. J.; Martinez, B.; Nair. S. K.; Nicolet, Y.; Rebuffat, S.; Sahl. H.-G.; Sareen, D.; Schmidt. E. W.; Schmitt. L.; Severinov, K.; Süssmuth, R. D.; Truman, A. W.; Wang, H.; Weng, J.-K.; Wezel. G. P. van; Zhang, Q.; Zhong, J.; Piel, J.; Mitchell, D. A.; Kuipers, O. P.; Donk, W. A. van der. New Developments in RiPP Discovery, Enzymology and Engineering. Nat Prod Rep 2021, 38 (1), 130-239. https://doi.org/10.1039/d0np00027b.
(26) Maksimov, M. O.; Pelczer, I.; Link, A. J. Precursor-Centric Genome-Mining Approach for Lasso Peptide Discovery. P Natl Acad Sci USA 2012, 109 (38), 15223-15228. https://doi.org/10.1073/pnas. 1208978109.
(27) Duquesne, S.; Destoumieux-Garzon, D.; Zirah, S.; Goulard, C.; Peduzzi, J.; Rebuffat, S. Two Enzymes Catalyze the Maturation of a Lasso Peptide in Escherichia coli. Chem Biol 2007, 14 (7), 793-803. https://doi.org/10.1016/j.chembiol.2007.06.004.
(28) Yan, K.-P.; Li. Y.; Zirah, S.; Goulard. C.; Knappe, T. A.; Marahiel. M. A.; Rebuffat, S. Dissecting the Maturation Steps of the Lasso Peptide Microcin J25 in Vitro. Chembiochem 2012, 13 (7), 1046-1052. https://doi.org/10.1002/cbic.201200016.
(29) Cheung-Lee, W. L.; Link, A. J. Genome Mining for Lasso Peptides: Past, Present, and Future. J Ind Microbiol Biotech 2019, 46 (9-10), 1371-1379. https://doi.org/10.1007/s10295-019-02197-z.
(30) Kloosterman, A. M.; Cimermancic, P.; Elsayed, S. S.; Du, C.; Hadjithomas, M.; Donia, M. S.; Fischbach, M. A.; Wezel, G. P. van; Medema, M. H. Expansion of RiPP Biosynthetic Space through Integration of Pan-Genomics and Machine Learning Uncovers a Novel Class of Lanthipeptides. PLOS Biol 2020, 18 (12), e3001026. https://doi.org/10.1371/journal.pbio.3001026.
(31) Agrawal, P.; Khater, S.; Gupta, M.; Sain, N.; Mohanty, D. RiPPMiner: A Bioinformatics Resource for Deciphering Chemical Structures of RiPPs Based on Prediction of Cleavage and Cross-Links. Nucleic Acids Res 2017, 45 (W1), W80-W88. https://doi.org/10.1093/nar/gkx408.
(32) Kloosterman, A. M.; Shelton, K. E.; Wezel, G. P. van; Medema, M. H.; Mitchell, D. A. RRE-Finder: A Genome-Mining Tool for Class-Independent RiPP Discovery. mSystems 2020, 5 (5), e00267-20. https://doi.org/10.1128/msystems.00267-20.
(33) Zhong. Z.; He. B.; Li, J.; Li, Y.-X. Challenges and Advances in Genome Mining of Ribosomally Synthesized and Post-Translationally Modified Peptides (RiPPs). Synthetic Syst Biotechnology 2020, 5 (3), 155-172. https://doi.org/10.1016/j.synbio.2020.06.002.
(34) de los Santos, E. L. C. NeuRiPP: Neural Network Identification of RiPP Precursor Peptides. Sci Rep 2019, 9 (1), 13406. https://doi.org/10.1038/s41598-019-49764-z.
(35) Russell, A. H.; Truman, A. W. Genome Mining Strategies for Ribosomally Synthesised and Post-Translationally Modified Peptides. Comput Struct Biotechnology J 2020, 18, 1838-1851. https://doi.org/10.1016/j.csbj.2020.06.032.
(36) Li, Y.; Rebuffat, S. The Manifold Roles of Microbial Ribosomal Peptide-Based Natural Products in Physiology and Ecology. J Biol Chem 2020, 295 (1), 34-54. https://doi.org/10.1074/jbc.rev119.006545.
(37) Rebuffat, S. Ribosomally Synthesized Peptides, Foreground Players in Microbial Interactions: Recent Developments and Unanswered Questions. Nat Prod Rep 2022, 39 (2), 273-310. https://doi.org/10.1039/d np00052g.
(38) Cao. L.; Do. T.; Link. A. J. Mechanisms of Action of Ribosomally Synthesized and Posttranslationally Modified Peptides (RiPPs). J Ind Microbiol Biot 2020, 48 (3-4), kuab005. https://doi.org/10.1093/jimb/kuab005.
(39) Granato, E. T.; Meiller-Legrand, T. A.; Foster, A. J. The Evolution and Ecology of Bacterial Warfare. Curr Biol 2019, 29 (11), R521-R537. https://doi.org/10.1016/j.cub.2019.04.024
(40) Braffman, N. R.; Piscotta, F. J.; Hauver, J.; Campbell, E. A.; Link, A. J.; Darst, S. A. Structural Mechanism of Transcription Inhibition by Lasso Peptides Microcin J25 and Capistruin. P Nail Acad Sci USA 2019, 116 (4), 1273-1278. https://doi.org/10.1073/pnas. 1817352116.
(41) Solbiati, J. O.; Ciaccio, M.; Farías, R. N.; González-Pastor, J. E.; Moreno, F.; Salomón, R. A. Sequence Analysis of the Four Plasmid Genes Required To Produce the Circular Peptide Antibiotic Microcin J25. J Bacteriol 1999, 181 (8), 2659-2662. https://doi.org/10.1128/jb.181.8.2659-2662.1999.
(42) Mukhopadhyay, J.; Sineva, E.; Knight, J.; Levy, R. M.; Ebright, R. H. Antibacterial Peptide Microcin J25 Inhibits Transcription by Binding within and Obstructing the RNA Polymerase Secondary Channel. Mol Cell 2004, 14 (6), 739-751. https://doi.org/10.1016/j.molcel.2004.06.010.
(43) Adelman, K.; Yuzenkova, J.; Porta, A. L.; Zenkin, N.; Lee, J.; Lis, J. T.; Borukhov, S.; Wang, M. D.; Severinov, K. Molecular Mechanism of Transcription Inhibition by Peptide Antibiotic Microcin J25. Mol Cell 2004, 14 (6), 753-762. https://doi.org/10.1016/j.molcel.2004.05.017.
(44) Kirsch, S. H.; Haeckl, F. P. J.; Muller, R. Beyond the Approved: Target Sites and Inhibitors of Bacterial RNA Polymerase from Bacteria and Fungi. Nat Prod Rep 2022, 39 (6), 226-263. https://doi.org/10.1039/d1np00067e.
(45) Pan, S. J.; Cheung, W. L.; Link, A. J. Engineered Gene Clusters for the Production of the Antimicrobial Peptide Microcin J25. Protein Express Purif 2010, 71 (2), 200-206. https://doi.org/10.1016/j.pep.2009.12.010.
(46) Hegemann, J. D.; Zimmermann, M.; Zhu, S.; Klug, D.; Marahiel, M. A. Lasso Peptides from Proteobacteria: Genome Mining Employing Heterologous Expression and Mass Spectrometry. Biopolymers 2013, 100 (5), 527-542. https://doi.org/10.1002/bip.22326.
(47) Koos, J. D.; Link, A. J. Heterologous and in Vitro econstitution of Fuscanodin, a Lasso Peptide from Thermobifida fusca. J Am Chem Soc 2019, 141 (2), 928-935. https://doi.org/10.1021/jacs.8b10724.
(48) Allen, C. D.; Chen, M. Y.; Trick, A. Y.; Le, D. T.; Ferguson, A. L.; Link, A. J. Thermal Unthreading of the Lasso Peptides Astexin-2 and Astexin-3. ACS Chem Biol 2016, 11 (11), 3043-3051. https://doi.org/10.1021/acschembio.6b00588.
(49) Hegemann, J. D. Factors Governing the Thermal Stability of Lasso Peptides. Chembiochem 2020, 21 (1-2), 7-8. https://doi.org/10.1002/cbic.201900364.
(50) Wilson, K.-A.; Kalkum, M.; Ottesen, J.; Yuzenkova, J.; Chait, B. T.; Landick, R.; Muir, T.; Severinov, K.; Darst, S. A. Structure of Microcin J25, a Peptide Inhibitor of Bacterial RNA Polymerase, Is a Lassoed Tail. J Am Chem Soc 2003, 125 (41), 12475-12483. https://doi.org/10.1002/ja036756q.
(51) Xie, X.; Marahiel, M. A. NMR as an Effective Tool for the Structure Determination of Lasso Peptides. Chembiochem 2012, 13 (5), 621-625. https://doi.org/10.1002/cbic.201100754.
(52) Fouque, K. J. D.; Hegemann, J. D.; Zirah, S.; Rebuffat, S.; Lescop, E.; Fernandez-Lima, F. Evidence of Cis/Trans-Isomerization at Pro7/Pro16 in the Lasso Peptide Microcin J25. J Am Soc Mass Spectr 2019, 30 (6), 1038-1045. https://doi.org/10.1007/s13361-019-02134-5.
(53) Fouque, K. J. D.; Lavanant, H.; Zirah, S.; Hegemann, J. D.; Fage, C. D.; Marahiel, M. A.; Rebuffat. S.; Afonso. C. General Rules of Fragmentation Evidencing Lasso Structures in CID and ETD. Analyst 2018, 143 (5), 1157-1170. https://doi.org/10.1039/c7an02052j.
(54) Güntert, P.; Buchner, L. Combined Automated NOE Assignment and Structure Calculation with CYANA. J Biomol NMR 2015, 62 (4), 453-471. https://doi.org/10.1007/s10858-015-9924-9.
(55) Bayro, M. J.; Mukhopadhyay, J.; Swapna, G. V. T.; Huang, J. Y.; Ma, L.-C.; Sineva, E.; Dawson, P. E.; Montelione, G. T.; Ebright, R. H. Structure of Antibacterial Peptide Microcin J25: A 21-Residue Lariat Protoknot. J Am Chem Soc 2003, 125 (41), 12382-12383. https://doi.org/10.1021/ja036677e.
(56) Rosengren, K. J.; Clark, R. J.; Daly, N. L.; Göransson, U.; Jones, A.; Craik, D. J. Microcin J25 Has a Threaded Sidechain-to-Backbone Ring Structure and Not a Head-to-Tail Cyclized Backbone. J Am Chem Soc 2003, 125 (41), 12464-12474. https://doi.org/10.1021/ja0367703.
(5) Hegemann, J. D.; Zimmermann, M.; Xie, X.; Marahiel, M. A. Caulosegnins I-III: A Highly Diverse Group of Lasso Peptides Derived from a Single Biosynthetic Gene Cluster. J Am Chem Soc 2013, 135 (1), 210-222. https://doi.org/10.1021/ja308173b.
(58) Hegemann, J. D.; Zimmermann. M.; Zhu, S.; Steuber, H.; Harms, K.; Xie, X.; 742 Marahiel, M. A. Xanthomonins I-III: A New Class of Lasso Peptides with a Seven-Residue Macrolactam Ring. Angew Chem Int Edit 2014, 53 (8), 2230-2234. https://doi.org/10.1002/anie.201309267.
(59) Son, S.; Jang, M.; Lee. B.; Hong, Y.-S.; Ko, S.-Y.; Jang, J.-H.; Ahn, J. S. Ulleungdin, a Lasso Peptide with Cancer Cell Migration Inhibitory Activity Discovered by the Genome Mining Approach. J Nat Prod 2018, 81 (10), 2205-2211. https://doi.org/10.1021/acs.jnatprod.8b00449.
(60) Tan, S.; Moore, G.; Nodwell, J. Put a Bow on It: Knotted Antibiotics Take Center Stage. Antibiotics (Basel) 2019, 8 (3), 117. https://doi.org/10.3390/antibiotics8030117.
(61) Brady, C.; Cleenwerck, I.; Venter. S.; Coutinho, T.; Vos, P. D. Taxonomic Evaluation of the Genus Enterobacter Based on Multilocus Sequence Analysis (MLSA): 756 Proposal to Reclassify E. nimipressuralis and E. amnigenus into Lelliottia Gen. Nov. as Lelliottia nimipressuralis Comb. Nov. and Lelliottia amnigena Comb. Nov., Respectively, E. gergoviae and E. pyrinus into Pluralibacter Gen. Nov. as Pluralibacter gergoviae Comb. Nov. and Pluralibacter pyrinus Comb. Nov., Respectively, E. cowanii, E. radicincitans, E. oryzae and E. arachidis into Kosakonia Gen. Nov. as Kosakonia cowanii Comb. Nov., Kosakonia radicincitans Comb. Nov., Kosakonia oryzae Comb. Nov. and Kosakonia arachidis Comb. Nov., Respectively, and E. turicensis, E. helveticus and E. pulveris into Cronobacter as Cronobacter zurichensis Nom. Nov., Cronobacter helveticus Comb. Nov. and Cronobacter pulveris Comb. Nov., Respectively, and Emended Description of the Genera Enterobacter and Cronobacter. Syst Appl Microbiol 2013, 36 (5), 309-319. https://doi.org/10.1016/j.syapm.2013.03.005.
(62) Zhu. B.; Lou, M.-M.; Xie, G.-L.; Wang, G.-F.; Zhou. Q.; Wang, F.; Fang, Y.; Su. T.; Li., B.; Duan. Y. P. Enterobacter mori sp. nov., associated with bacterial wilt on Morus alba L. Int J Syst Evol Micr 2011, 61 (Pt 11), 2769-2774. https://doi.org/10.1099/ijs 0.028613-0.
(63) Koth, K.; Boniface, J.; Chance, E. A.; Hanes, M. C. Enterobacter asburiae and Aeromonas hydrophila: Soft Tissue Infection Requiring Debridement. Orthopedics 2012, 35 (6), e996-9. https://doi.org/10.3928/01477447-20120525-52.
(64) Westerfeld, C.; Papaliodis, G. N.; Behlau, I.; Durand, M. L.; Sobrin L. Enterobacter amnigenus Endophthalmitis. Retin Cases Brief Rep 2009, 3 (4), 409-411. https://doi.org/10.1097/icb.0b013e31818a46c0.
(65) Xue, Y.; Hu, M.; Chen, S.; Hu, A.; Li, S.; Han, H.; Lu, G.; Zeng, L.; Zhou, J. Enterobacter asburiae and Pantoea ananatis Causing Rice Bacterial Blight in China. Plant Dis 2021, 105 (8), 2078-2088. https://doi.org/10.1094/pdis-10-20-2292-re.
(66) Ren, Y.; Ren, Y.; Zhou, Z.; Guo, X.; Li. Y., Feng, L.; Wang. L. Complete Genome Sequence of Enterobacter cloacae subsp. cloacae Type Strain ATCC 13047 J Bacteriol 2010, 192 (9), 2463-2464. https://doi.org/10.1128/jb.00067·10.
(67) Salamzade, R.; Manson, A. L.; Walker, B. J.; Brennan-Krohn, T.; Worby. C. J.; Ma, P.; He, L. L.; Shea, T. P.; Qu, J.; Chapman, S. B.; Howe, W.; Young, S. K.; Wurster, J. I.; Delaney, M. L.; Kanjilal, S.; Onderdonk, A. B.; Bittencourt, C. E.; Gussin, G. M.; Kim, D.; Peterson, E. M.; Ferraro, M. J.; Hooper, D. C.; Shenoy, E. S.; Cuomo, C. A.; Cosimi, L. A.; Huang, S. S.; Kirby, J. E.; Pierce, V. M.; Bhattacharyya, R. P.; Earl, A. M. Inter-Species Geographic Signatures for Tracing Horizontal Gene Transfer and Long-Term Persistence of Carbapenem Resistance. Genome Med 2022, 14 (1), 37. https://doi.org/10.1186/s13073-022-01040-y.
(68) Do, T.; Thokkadam. A.; Leach, R.; Link, A. J. Phenotype-Guided Comparative Genomics Identifies the Complete Transport Pathway of the Antimicrobial Lasso Peptide Ubonodin in Burkholderia. ACS Chem Biol 2022. https://doi.org/10.1021/acschembio.2c00420.
(69) DiCaprio, A. J.; Firouzbakht, A.; Hudson, G. A.; Mitchell, D. A. Enzymatic Reconstitution and Biosynthetic Investigation of the Lasso Peptide Fusilassin. J Am Chem Soc 2019, 141 (1), 290-297. https://doi.org/10.1021/jacs.8b09928.
(70) Pavlova, O.; Mukhopadhyay, J.; Sineva, E.; Ebright, R. H.; Severinov, K. Systematic Structure-Activity Analysis of Microcin J25. J Biol Chem 2008, 283 (37), 25589-25595. https://doi.org/10.1074/jbc.m803995200.
(71) Knappe, T. A.; Linne, U.; Robbel, L.; Marahiel, M. A. Insights into the Biosynthesis and Stability of the Lasso Peptide Capistruin. Chem Biol 2009, 16 (12), 1290-1298. https://doi.org/10.1016/j.chembiol.2009.11.009.
(72) Liu, T.; Ma, X.; Yu, J.; Yang, W.; Wang. G.; Wang, Z.; Ge, Y.; Song, J.; Han, H.; Zhang, W.; Yang, D.; Liu, X.; Ma, M. ational Generation of Lasso Peptides Based on Biosynthetic Gene Mutations and Site-Selective Chemical Modifications. Chem Sci 2021, 12 (37), 12353-12364. https://doi.org/10.1039/d sc02695j.
(73) Zimmermann, M.; Hegemann, J. D.; Xie, X.; Marahiel, M. A. The Astexin-1 Lasso Peptides: Biosynthesis, Stability, and Structural Studies. Chem Biol 2013, 20 (4), 558-569. https://doi.org/10.1016/j.chembiol.2013.03.013.
(74) Hegemann, J. D.; Fage. C. D.; Zhu. S.; Harms. K.; Leva. F. S. D.; Novellino, E. Marinelli, L.; Marahiel, M. A. The Ring Residue Proline 8 Is Crucial for the Thermal Stability of the Lasso Peptide Caulosegnin II. Mol Biosyst 2016, 12 (4), 1106-1109. https://doi.org/10.1039/c6mb00081a.
(75) Schröder, H. V; Zhang, Y.; Link. A. J. Dynamic Covalent Self-Assembly of Mechanically Interlocked Molecules Solely Made from Peptides. Nat Chem 2021, 13 (9), 850-857. https://doi.org/10.1038/s41557-021-00770-7.
(76) Madeira, F.; Pearce, M.; Tivey, A. R. N.; Basutkar, P.; Lee, J.; Edbali, O.; Madhusoodanan, N.; Kolesnikov, A.; Lopez. R. Search and Sequence Analysis Tools Services from EMBL-EBI in 2022. Nucleic Acids Res 2022, 50 (W1), W276-W279. https://doi.org/10.1093/nar/gkac240.
(77) Hoover, D. M.; Lubkowski, J. DNAWorks: An Automated Method for Designing Oligonucleotides for PCR-Based Gene Synthesis. Nucleic Acids Res 2002, 30 (10), e43-e43. https://doi.org/10.1093/nar/30.10.e43.
(78) Gill, S. C.; Hippel. P. H. von. Calculation of Protein Extinction Coefficients from Amino Acid Sequence Data. Anal Biochem 1989, 182 (2), 319-326. https://doi.org/10.1016/0003-2697 (89) 90602-7.
(79) Carson, D. V.; Patiño, M.; Elashal, H. E.; Cartagena, A. J.; Zhang, Y.; Whitley, M. E.; So, L.; Kayser-Browne, A. K.; Earl. A. M.; Bhattacharyya, R. P.; Link. A. J. Cloacaenodin, an Antimicrobial Lasso Peptide with Activity against Enterobacter. ACS Infect. Dis. 2023, 9 (1), 111-121. https://doi.org/10.1021/acsinfecdis.2c00446.
(80) Fontes-Perez. H.; Olvera-García. M.; Chávez-Martínez, A.; Rodriguez-Almeida, F. A.; Arzola-Alvarez, C. A.; Sanchez-Flores. A.; Corral-Luna, A. Genome Sequence of Citrobacter Sp. CtB7.12. Isolated from the Gut of the Desert Subterranean Termite Heterotermes aureus. Genome Announc. 2015, 3 (6), e01290-15. https://doi.org/10.1128/genomea.01290-15.
(81) CDC & FDA Antibiotic Resistance Isolate Bank. Atlanta (GA): CDC. 22 Aug. 2023.

1. A method for inhibiting growth of a microorganism, comprising
providing a cloacaenodin-class lasso peptide that is purified; and
exposing the microorganism to the cloacaenodin-class lasso peptide,
so that the growth of the microorganism is inhibited,
wherein the cloacaenodin-class lasso peptide comprises a ring, a loop region, and a tail region,
wherein the ring is bonded to the loop region,
wherein the loop region is bonded to the tail region,
wherein the cloacaenodin-class lasso peptide comprises a peptide sequence
[SEQ ID NO. 1]
GHSVDRIPEYFGPPGLPGPVLFYS,
wherein 0, 1, or 2 of residues 2 through 8 of the peptide sequence are removed or replaced with another residue,
wherein 0 or 1 residue is inserted after one of residues 1 through 8,
wherein 0, 1, 2, 3, 4, or 5 of residues 12 through 24 of the peptide sequence are removed or replaced with another residue, and
wherein 0, 1, 2, 3, or 4 residues are inserted after at least one of residues 11 through 21.

2. The method according to claim 1, wherein the cloacaenodin-class lasso peptide is not cloacaenodin of the peptide sequence GHSVDRIPEYFGPPGLPGPVLFYS [SEQ ID NO. 1], not cloacaenodin-2 of the peptide sequence GHSVDRIPEYFGPPGLPSPVLFYS [SEQ ID NO. 105], and not cloacaenodin-3 of the peptide sequence GHSVDRIPEYFGPPGGPILFYS [SEQ ID NO. 106].

3. The method according to claim 1, wherein the ring is of 9 residues.

4. The method according to claim 1, wherein the ring is of a subsequence GHSVDRIPE [SEQ ID NO. 9].

5. The method according to claim 1, wherein the ring is of 10 residues.

6. The method according to claim 1, wherein the ring is of a subsequence GHSVADRIPE [SEQ ID NO. 7].

7. The method according to claim 1, wherein the loop region is of 13 residues.

8. The method according to claim 1, wherein the loop region is of a subsequence
[SEQ ID NO. 115]
YFGPPGLPGPVLF.

9. The method according to claim 1, wherein the loop region is of 12 residues or 11 residues.

10. The method according to claim 1, wherein the tail region is of 2 residues.

11. The method according to claim 1, wherein the tail region is of a subsequence YS.

12. The method according to claim 1, wherein the cloacaenodin-class lasso peptide is threaded.

13. The method according to claim 1, wherein residues 22 through 24 of the peptide sequence are not removed or replaced.

14. The method according to claim 1, wherein at most 2 of residues 12 through 21 of the peptide sequence are removed or replaced.

15. The method according to claim 1, wherein residue 8 of the peptide sequence is proline (P).

16. The method according to claim, wherein residue 8 of the peptide sequence is replaced by alanine (A).

17. The method according to claim 1, wherein residue 4 of the peptide sequence is replaced by proline (P).

18. The method according to claim 1, wherein residue 22 of the peptide sequence is replaced by tryptophan (W).

19. The method according to claim 1, wherein residue 23 of the peptide sequence is replaced by tryptophan (W).

20. The method according to claim 1, wherein residue 24 of the peptide sequence is replaced by alanine (A).

21. The method according to claim 1, wherein residue 24 of the peptide sequence is replaced by tyrosine (Y).

22. The method according to claim 1, wherein residue 24 of the peptide sequence is replaced by threonine (T).

23. The method according to claim 1, wherein residue 24 of the peptide sequence is replaced by cysteine (C).

24. The method according to claim 1, wherein residue 10 of the peptide sequence is replaced by alanine (A).

25. The method according to claim 1, wherein alanine (A) is inserted after residue 4 of the peptide sequence.

26. The method according to claim 1, wherein residue 18 of the peptide sequence is serine (S).

27. The method according to claim 1, wherein residue 20 of the peptide sequence is isoleucine (I).

28. The method according to claim 1, wherein residues 16 and 17 of the peptide sequence are removed.

29. The method according to claim 1, wherein the cloacaenodin-class lasso peptide is cloacaenodin of a peptide sequence GHSVDRIPEYFGPPGLPGPVLFYS [SEQ ID NO. 1].

30. The method according to claim 1, wherein the cloacaenodin-class lasso peptide is cloacaenodin-2 of a peptide sequence GHSVDRIPEYFGPPGLPSPVLFYS [SEQ ID NO. 105].

31. The method according to claim 1, wherein the cloacaenodin-class lasso peptide is cloacaenodin-3 of a peptide sequence GHSVDRIPEYFGPPGGPILFYS [SEQ ID NO. 106].

32. The method according to claim 1, wherein the cloacaenodin-class lasso peptide is of a peptide sequence GHSVDRIPEYFGPPGLPGPVLWYS [SEQ ID NO. 111].

33. The method according to claim 1, wherein the cloacaenodin-class lasso peptide is of a peptide sequence GHSVDRIPEYFGPPGLPGPVLFYA [SEQ ID NO. 113].

34. The method according to claim 1, wherein the cloacaenodin-class lasso peptide is of a peptide sequence GHSVDRIPEYFGPPGLPGPVLFYY [SEQ ID NO. 114].

35. The method according to claim 1, wherein the cloacaenodin-class lasso peptide is of a peptide sequence selected from the group consisting of
[SEQ ID NO. 67]
GHSVADRIPEYFGPPGLPGPVLFYS,
[SEQ ID NO. 109]
GHSVDRIAEYFGPPGLPGPVLFYS,
[SEQ ID NO. 110]
GHSPDRIPEYFGPPGLPGPVLFYS,
[SEQ ID NO. 112]
GHSVDRIPEYFGPPGLPGPVLFWS,
[SEQ ID NO. 116]
GHSVDRIPEYFGPPGLPGPVLFYT,
[SEQ ID NO. 117]
GHSVDRIPEYFGPPGLPGPVLFYC,
and
[SEQ ID NO. 118]
GHSVDRIPEAFGPPGLPGPVLFYS.

36. The method according to claim 1, wherein the peptide sequence is at least 85% homologous to GHSVDRIPEYFGPPGLPGPVLFYS [SEQ ID NO. 1].

37. The method according to claim 1, wherein the peptide sequence is at least 95% homologous to GHSVDRIPEYFGPPGLPGPVLFYS [SEQ ID NO. 1].

38. The method according to claim 1,
wherein the ring is of 9 residues and
wherein the cloacaenodin-class lasso peptide comprises a threaded structure.

39. The method according to claim 1, wherein the microorganism is a gram-negative gammaproteobacterium.

40. The method according to claim 1, wherein the microorganism is of order Enterobacterales.

41. The method according to claim 1, wherein the microorganism is of family Enterobacteriaceae.

42. The method according to claim 1, wherein the microorganism is a species of Enterobacter.

43. The method according to claim 1, wherein the microorganism is Enterobacter amnigenus, Enterobacter asburiae, Enterobacter mori, or Enterobacter nimipressuralis.

44. The method according to claim 1, wherein the microorganism is Enterobacter cloacae.

45. The method according to claim 1, wherein the microorganism is Enterobacter hormaechei, Enterobacter kobei, or Enterobacter ludwigii.

46. The method according to claim 1, wherein the microorganism is Enterobacter xiangfangensis.

47. The method according to claim 1, wherein the microorganism is a species of Kluyvera.

48. The method according to claim 1, wherein the microorganism is Kluyvera ascorbata.

49. The method according to claim 1, wherein the microorganism is resistant to an antibiotic, resistant to a broad spectrum antibiotic, resistant to an antibiotic of last resort, or resistant to a beta-lactam antibiotic.

50. The method according to claim 1, wherein the microorganism is resistant to a carbapenem.

51. The method according to claim 1,
wherein providing the cloacaenodin-class lasso peptide comprises providing a pharmaceutical composition comprising the cloacaenodin-class lasso peptide and
wherein the pharmaceutical composition further comprises a pharmaceutically acceptable carrier or diluent.

52. The method according to claim 51, wherein the lasso peptide is present in the pharmaceutical composition at a concentration of less than 10 μM.

53. The method according to claim 51, wherein the pharmaceutical composition is of a dosage form selected from the group consisting of an injectable liquid, a capsule, a tablet, a pill, a suppository, a powder, a time-release capsule, a time-release table, a time release pill, a time-release suppository, a cream, an ointment, a gel, and an impregnated wound dressing.

54. The method according to claim 1, wherein the microorganism is exposed to the cloacaenodin-class lasso peptide in vitro.

55. The method according to claim 1, wherein the cloacaenodin-class lasso peptide is of a minimal inhibitory concentration (MIC) against the microorganism of 8 μM or less.

56. The method according to claim 1, wherein the cloacaenodin-class lasso peptide is of a minimal inhibitory concentration (MIC) against the microorganism of 4 μM or less.

57. The method according to claim 1, wherein the cloacaenodin-class lasso peptide is of a minimal inhibitory concentration (MIC) against the microorganism of 2 μM or less.

58. The method according to claim 1, wherein the microorganism is exposed to the cloacaenodin-class lasso peptide within or on a patient.

59. A method of treating a patient infected with the microorganism, comprising administering the cloacaenodin-class lasso peptide to the patient according to the method of claim 1, thereby treating the patient.

60. A method of treating a patient to prevent infection with the microorganism, comprising administering the cloacaenodin-class lasso peptide to the patient according to the method of claim 1, thereby preventing infection of the patient with the microorganism.

61. The method of treating a patient according to claim 59, wherein the cloacaenodin-class lasso peptide is administered to the patient intravenously, intraperitoneally, intramuscularly, subcutaneously, intradermally, by injection into tissue, orally, by nasal insufflation, by inhalation, topically, vaginally, urethrally, or rectally.

62. A purified cloacaenodin-class lasso peptide, comprising
a peptide sequence GHSVDRIPEYFGPPGLPGPVLFYS [SEQ ID NO. 1],
wherein the peptide sequence comprises a ring, a loop region, and a tail region,
wherein the ring is bonded to the loop region,
wherein the loop region is bonded to the tail region,
wherein 0, 1, or 2 of residues 2 through 8 of the peptide sequence are removed or replaced with another residue,
wherein 0 or 1 residue is inserted after one of residues 1 through 8,
wherein 0, 1, 2, 3, 4, or 5 of residues 12 through 24 of the peptide sequence are removed or replaced, and
wherein 0, 1, 2, 3, or 4 residues are inserted after residues 11 through 21.

63. The purified cloacaenodin-class lasso peptide according to claim 62, wherein the purified cloacaenodin-class lasso peptide is not cloacaenodin of the peptide sequence GHSVDRIPEYFGPPGLPGPVLFYS [SEQ ID NO. 1], not cloacaenodin-2 of the peptide sequence GHSVDRIPEYFGPPGLPSPVLFYS [SEQ ID NO. 105], and not cloacaenodin-3 of the peptide sequence GHSVDRIPEYFGPPGGPILFYS [SEQ ID NO. 106].

64. The purified cloacaenodin-class lasso peptide according to claim 62, wherein the ring is of 9 residues.

65. The purified cloacaenodin-class lasso peptide according to claim 62 wherein the ring comprises a subsequence GHSVDRIPE [SEQ ID NO. 9].

66. The purified cloacaenodin-class lasso peptide according to claim 62, wherein the ring is of 10 residues.

67. The purified cloacaenodin-class lasso peptide according to claim 62, wherein the ring comprises a subsequence GHSVADRIPE [SEQ ID NO. 7].

68. The purified cloacaenodin-class lasso peptide according to claim 62, wherein the loop region is of 13 residues.

69. The purified cloacaenodin-class lasso peptide according to claim 62, wherein the loop region comprises a subsequence YFGPPGLPGPVLF [SEQ ID NO. 115].

70. The purified cloacaenodin-class lasso peptide according to claim 62, wherein the loop region is of 12 residues or 11 residues.

71. The purified cloacaenodin-class lasso peptide according to claim 62, wherein the tail region is of 2 residues.

72. The purified cloacaenodin-class lasso peptide according to claim 62, wherein the tail region comprises a subsequence YS.

73. The purified cloacaenodin-class lasso peptide according to claim 62,
wherein the tail region is threaded through the ring and
wherein a residue of the tail region that is bonded to the loop region and a residue of the loop region that is bonded to the tail region are on opposite sides of the ring.

74. The purified cloacaenodin-class lasso peptide according to claim 62, wherein the tail region of a subsequence YS is threaded through the ring of a subsequence GHSVDRIPE [SEQ ID NO. 9].

75. The purified cloacaenodin-class lasso peptide according to claim 62, wherein residues 22 through 24 of the peptide sequence are not removed or replaced.

76. The purified cloacaenodin-class lasso peptide according to claim 62, wherein at most 2 of residues 12 through 21 of the peptide sequence are removed or replaced.

77. The purified cloacaenodin-class lasso peptide according to claim 62, wherein residue 8 of the peptide sequence is proline (P).

78. The purified cloacaenodin-class lasso peptide according to claim 62, wherein residue 8 of the peptide sequence is alanine (A).

79. The purified cloacaenodin-class lasso peptide according to claim 62, wherein residue 4 of the peptide sequence is proline (P).

80. The purified cloacaenodin-class lasso peptide according to claim 62, wherein residue 22 of the peptide sequence is tryptophan (W).

81. The purified cloacaenodin-class lasso peptide according to claim 62, wherein residue 23 of the peptide sequence is tryptophan (W).

82. The purified cloacaenodin-class lasso peptide according to claim 62, wherein residue 24 of the peptide sequence is alanine (A).

83. The purified cloacaenodin-class lasso peptide according to claim 62, wherein residue 24 of the peptide sequence is tyrosine (Y).

84. The purified cloacaenodin-class lasso peptide according to claim 62, wherein residue 24 of the peptide sequence is threonine (T).

85. The purified cloacaenodin-class lasso peptide according to claim 62, wherein residue 24 of the peptide sequence is cysteine (C).

86. The purified cloacaenodin-class lasso peptide according to claim 62, wherein residue 10 of the peptide sequence is alanine (A).

87. The purified cloacaenodin-class lasso peptide according to claim 62, wherein alanine (A) is inserted after residue 4 in the peptide sequence.

88. The purified cloacaenodin-class lasso peptide according to claim 62, wherein residue 18 of the peptide sequence is serine (S).

89. The purified cloacaenodin-class lasso peptide according to claim 62, wherein residue 20 of the peptide sequence is isoleucine (I).

90. The purified cloacaenodin-class lasso peptide according to claim 62, wherein one or both of residues 16 and 17 of the peptide sequence are removed.

91. The purified cloacaenodin-class lasso peptide according to claim 62, wherein the peptide sequence is GHSVDRIPEYFGPPGLPGPVLFYS [SEQ ID NO. 1]

92. The purified cloacaenodin-class lasso peptide according to claim 62, wherein the peptide sequence is GHSVDRIPEYFGPPGLPSPVLFYS [SEQ ID NO. 105].

93. The purified cloacaenodin-class lasso peptide according to claim 62, wherein the peptide sequence is GHSVDRIPEYFGPPGGPILFYS [SEQ ID NO. 106].

94. The purified cloacaenodin-class lasso peptide according to claim 62, wherein the peptide sequence is GHSVDRIPEYFGPPGLPGPVLWYS [SEQ ID NO. 111].

95. The purified cloacaenodin-class lasso peptide according to claim 62, wherein the peptide sequence is GHSVDRIPEYFGPPGLPGPVLFYA [SEQ ID NO. 113].

96. The purified cloacaenodin-class lasso peptide according to claim 62, wherein the peptide sequence is GHSVDRIPEYFGPPGLPGPVLFYY [SEQ ID NO. 114].

97. The purified cloacaenodin-class lasso peptide according to claim 62, wherein the peptide sequence is selected from the group consisting of GHSVADRIPEYFGPPGLPGPVLFYS [SEQ ID NO. 67],
[SEQ ID NO. 109]
GHSVDRIAEYFGPPGLPGPVLFYS,
[SEQ ID NO. 110]
GHSPDRIPEYFGPPGLPGPVLFYS,
[SEQ ID NO. 112]
GHSVDRIPEYFGPPGLPGPVLFWS,
[SEQ ID NO. 116]
GHSVDRIPEYFGPPGLPGPVLFYT,
[SEQ ID NO. 117]
GHSVDRIPEYFGPPGLPGPVLFYC,
and
[SEQ ID NO. 118]
GHSVDRIPEAFGPPGLPGPVLFYS.

98. The purified cloacaenodin-class lasso peptide according to claim 62, wherein the peptide sequence is at least 85% homologous to GHSVDRIPEYFGPPGLPGPVLFYS [SEQ ID NO. 1].

99. The purified cloacaenodin-class lasso peptide according to claim 62, wherein the peptide sequence is at least 95% homologous to GHSVDRIPEYFGPPGLPGPVLFYS [SEQ ID NO. 1].

100. A pharmaceutical composition comprising the purified cloacaenodin-class lasso peptide according to claim 62 and a pharmaceutically acceptable carrier or diluent.

101. The pharmaceutical composition according to claim 100 of a dosage form selected from the group consisting of an injectable liquid, a capsule, a tablet, a pill, a suppository, a powder, a time-release capsule, a time-release tablet, a time release pill, a time-release suppository, a cream, an ointment, a gel, or an impregnated wound dressing.

102. The purified cloacaenodin-class lasso peptide according to claim 62 for use as a medicament.

103. The purified cloacaenodin-class lasso peptide according to claim 62 for use in treatment of an infection with a microorganism.

104. The purified cloacaenodin-class lasso peptide according to claim 62 for use in prevention of an infection with a microorganism.

105. The use of the purified cloacaenodin-class lasso peptide according to claim 62 in the manufacture of a medicament for treatment of an infection with a microorganism.

106. The use of the purified cloacaenodin-class lasso peptide according to claim 62 in the manufacture of a medicament for prevention of an infection with a microorganism.

107. The purified cloacaenodin-class lasso peptide according to claim 103, wherein the microorganism is a gammaproteobacterium.

108. The purified cloacaenodin-class lasso peptide according to claim 103, wherein the microorganism is of order Enterobacterales.

109. The purified cloacaenodin-class lasso peptide according to claim 103, wherein the microorganism is of family Enterobacteriaceae.

110. The purified cloacaenodin-class lasso peptide according to claim 103, wherein the microorganism is of genus Enterobacter.

111. The purified cloacaenodin-class lasso peptide according to claim 103, wherein the microorganism is Enterobacter amnigenus, Enterobacter asburiae, Enterobacter mori, or Enterobacter nimipressuralis.

112. The purified cloacaenodin-class lasso peptide according to claim 103, wherein the microorganism is Enterobacter cloacae.

113. The purified cloacaenodin-class lasso peptide according to claim 103, wherein the microorganism is Enterobacter hormaechei, Enterobacter kobei, or Enterobacter ludwigii.

114. The purified cloacaenodin-class lasso peptide according to claim 103, wherein the microorganism is Enterobacter xiangfangensis.

115. The purified cloacaenodin-class lasso peptide according to claim 103, wherein the microorganism is of genus Kluyvera.

116. The purified cloacaenodin-class lasso peptide according to claim 103, wherein the microorganism is Kluyvera ascorbata.

117. A method of producing a cloacaenodin-class lasso peptide, comprising
refactoring the precursor, protease, cyclase, and exporter genes for cloacaenodin into a plasmid in vitro,
transforming the plasmid into cells in vitro,
growing the cells in vitro,
inducing expression of the cloacaenodin-class lasso peptide by the cells in vitro,
separating the cells and a supernatant, and
obtaining purified cloacaenodin-class lasso peptide from the supernatant.

118. The method of producing a cloacaenodin-class lasso peptide according to claim 117, further comprising freezing the purified cloacaenodin-class lasso peptide at −10° C. or less within 30 minutes of obtaining the purified cloacaenodin-class lasso peptide from the supernatant.

119. The method of producing a cloacaenodin-class lasso peptide according to claim 118, wherein the purified cloacaenodin-class lasso peptide is frozen at −20° C. or less.

120. The method of producing a cloacaenodin-class lasso peptide according to claim 118, wherein the purified cloacaenodin-class lasso peptide is frozen at −80° C. or less.

121. The method of producing a cloacaenodin-class lasso peptide according to claim 117, wherein the purified cloacaenodin-class lasso peptide is cloacaenodin of a peptide sequence
[SEQ ID NO. 1]
GHSVDRIPEYFGPPGLPGPVLFYS.

122. The method of producing a cloacaenodin-class lasso peptide according to claim 117, further comprising
using site-directed mutagenesis to modify the plasmid,
wherein the purified cloacaenodin-class lasso peptide comprises a peptide sequence
[SEQ ID NO. 1]
GHSVDRIPEYFGPPGLPGPVLFYS,
wherein 0, 1, or 2 of residues 2 through 8 of the peptide sequence are removed or replaced with another residue,
wherein 0 or 1 residue is inserted after one of residues 1 through 8,
wherein 0, 1, 2, 3, 4, or 5 of residues 12 through 24 of the peptide sequence are removed or replaced, and
wherein 0, 1, 2, 3, or 4 residues are inserted after at least one of residues 11 through 21.

123. The method of producing a cloacaenodin-class lasso peptide according to claim 122, wherein the purified cloacaenodin-class lasso peptide is cloacaenodin-2 of a peptide sequence GHSVDRIPEYFGPPGLPSPVLFYS [SEQ ID NO. 105] or cloacaenodin-3 of a peptide sequence GHSVDRIPEYFGPPGGPILFYS [SEQ ID NO. 106].

124. The method of producing a cloacaenodin-class lasso peptide according to claim 122, wherein the purified cloacaenodin-class lasso peptide is of a peptide sequence selected from the group consisting of
[SEQ ID NO. 67]
GHSVADRIPEYFGPPGLPGPVLFYS,
[SEQ ID NO. 109]
GHSVDRIAEYFGPPGLPGPVLFYS,
[SEQ ID NO. 110]
GHSPDRIPEYFGPPGLPGPVLFYS,
[SEQ ID NO. 111]
GHSVDRIPEYFGPPGLPGPVLWYS,
[SEQ ID NO. 112]
GHSVDRIPEYFGPPGLPGPVLFWS,
[SEQ ID NO. 113]
GHSVDRIPEYFGPPGLPGPVLFYA,
[SEQ ID NO. 114]
GHSVDRIPEYFGPPGLPGPVLFYY,
[SEQ ID NO. 116]
GHSVDRIPEYFGPPGLPGPVLFYT,
[SEQ ID NO. 117]
GHSVDRIPEYFGPPGLPGPVLFYC,
and
[SEQ ID NO. 118]
GHSVDRIPEAFGPPGLPGPVLFYS.
